(19) United States
(12) Patent Application Publication
(10) Pub. No.: US 2025/0129073 A1
(43) Pub. Date: Apr. 24, 2025

(54) INHIBITOR OF RECEPTOR-INTERACTING PROTEIN KINASE 1, AND PREPARATION METHOD AND USE THEREFOR

(71) Applicant: CHINA PHARMACEUTICAL UNIVERSITY, Nanjing, Jiangsu (CN)

(72) Inventors: Jinxin Wang, Nanjing, Jiangsu (CN); Ao Niu, Nanjing, Jiangsu (CN); Lizhi Lin, Nanjing, Jiangsu (CN); Wenjia Zhou, Nanjing, Jiangsu (CN); Junwei Shi, Nanjing, Jiangsu (CN); Minghui Wang, Nanjing, Jiangsu (CN)

(21) Appl. No.: 18/694,439

(22) PCT Filed: Nov. 14, 2022
(86) PCT No.: PCT/CN2022/131601

§ 371 Date: Mar. 22, 2024

(30) Foreign Application Priority Data
Sep. 22, 2021 (CN) 202111105717.9

(51) Int. Cl. C07D 471/10; A61K 31/438; A61K 31/444; A61K 31/501; A61K 31/506

(52) U.S. Cl. CPC C07D 471/10; A61K 31/438; A61K 31/444; A61K 31/501; A61K 31/506

(57) ABSTRACT

The invention belongs to the field of pharmacology, and particularly relates to an inhibitor of a receptor-interacting protein kinase 1, and a preparation method and use therefor. Specifically provided is a spiro compound having a structure represented by general formula (I), or a pharmaceutically acceptable salt or stereoisomer thereof, or a prodrug thereof. In the present invention, a series of spiro compounds are synthesized for the first time, the series of compounds are novel compounds, and the preparation method is also a brand-new reaction route. The method has the effect of inhibiting the kinase activity of RIPK1, and can be used as a drug or a prodrug for preventing and/or treating RIPK1-related diseases.

### TECHNICAL FIELD

The present disclosure belongs to the field of pharmacology and specifically relates to a novel inhibitor of receptor interacting protein kinase 1 and preparation method and use thereof.

### BACKGROUND

There are different forms of cell death, which can be morphologically divided into apoptosis and necrosis. In the past, it was thought that cell necrosis was an accidental and unregulated unprogrammed cell death. However, in recent years, a series of cell death patterns such as necroptosis, pyroptosis, and ferroptosis have morphological characteristics of necrosis and are regulated by cellular signaling. Unlike apoptosis, necrotic cell death can lead to the release of cell contents, particularly damage associated molecular patterns (DAMPs), which can be recognized by pattern recognition receptors in adjacent cells, activating the adaptive immune system and mediating inflammation (Immunity 2013, 38 (2), 209-223). Among necrotic cell death modes, necroptosis has been studied deeply in recent years.

It has been reported that receptor interacting protein kinase 1 (RIPK1) and receptor-interacting protein kinase 3 (RIPK3) are key regulatory proteins in necroptosis. RIPK1 is a serine/threonine protein kinase that is involved in downstream cell signaling when cells are stimulated by external signals such as FASL (J. Cell Biol. 2009, 187 (7), 1037-1054), TRAIL (J. Cell Biol. 2009, 187 (7), 1037-1054), and TNF-α (Nat. Chem. Biol. 2008, 4 (5), 313-321). RIPK1 not only provides a binding framework for other signal factors, but more importantly, the activation of its kinase activity is also necessary for necroptosis. Therefore, inhibition of RIPK1 kinase activity can block the occurrence of necroptosis.

Existing studies have shown that necroptosis plays an important role in the pathological processes of various diseases, including ischemic stroke (Nat. Chem. Biol. 2005, 1 (2), 112-119), Alzheimer's disease (Nat. Neurosci. 2017, 20 (9), 1236-1246), and amyotrophic lateral sclerosis (Neoptosis Drivers Motor Neuron Death in Models of Both Sports and Family ALS. Neuron 2014, 81 (5), 1001-1008.) Inflammatory bowel disease (Nature 2011, 477 (7364), 330-334.), psoriasis (Immunity 2011, 35 (4), 572-582), non-alcoholic steatohepatitis (Clin. Sci. 2015, 129 (8), 721-739), etc.

Therefore, the effective and selective inhibitors of RIPK1 kinase which block the occurrence of necroptosis, could provide therapeutic effects for diseases related to DAMPs release, cell death, or inflammation.

### CONTENT OF THE PRESENT INVENTION

The purpose of the present disclosure is to provide a novel spirocyclic compound that has inhibitory activity against human Receptor-interacting Protein Kinase 1 (RIPK1) and necroptosis.

The first aspect of the present disclosure is to provide a spirocyclic compound having a structure of Formula (I), or a pharmaceutically acceptable salt, or a stereoisomer, or a prodrug thereof:

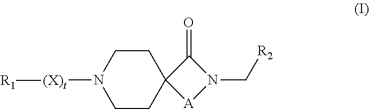

wherein, A is selected from:
C₁-C₆ cycloalkyl with 0-3 heteroatoms, and C₂-C₅ cycloalkenyl with 0-3 heteroatoms;
t is 0 or 1;
X is selected from:
1) CH₂, C₁-C₆ alkyl or cycloalkyl, C₁-C₆ haloalkyl, C₁-C₆ alkoxy;
2) —CO—, —SO₂—, —(CH₂)ₙCO—, —(CH₂)ₙSO₂—, —NHCO—, wherein, n is 1, 2, 3 or 4;
wherein, R₁ is selected from:
1) H;
2) substituted and unsubstituted C₆-C₁₀ aryl and 4-10 membered heteroaryl; or
3) C₁-C₄ alkyl, C₃-C₇ cycloalkyl, substituted and unsubstituted 3-8 membered heterocyclyl;
R₂ is selected from:
1) substituted and unsubstituted C₆-C₁₀ aryl, substituted and unsubstituted 4-10 membered heteroaryl;
2) C₁-C₄ alkyl, C₃-C₇ cycloalkyl, substituted and unsubstituted 3-8 membered heterocyclyl.

Each of the 3-8 membered heterocyclyl, C₆-C₁₀ aryl, and 4-10 membered heteroaryl is independently optionally substituted by substituents selected from the group consisting of halogen, amino, nitro, trifluoromethyl, difluoromethyl, cyano, hydroxyl, —C(O)C₁-C₆ alkyl, C₁-C₆ alkyl, C₁-C₆ haloalkyl, C₁-C₆ alkoxy, and C₁-C₆ haloalkoxy.

In the present disclosure, the “pharmaceutically acceptable salt” includes pharmaceutically acceptable acid addition salt and pharmaceutically acceptable base addition salt. “Pharmaceutically acceptable acid addition salt” refers to a salt that maintains the biological effectiveness of the free base without other adverse effects, which is formed with inorganic or organic acid. Inorganic acid salt includes but is not limited to hydrochloride, hydrobromate, sulfate, phosphate, etc.; organic acid salt includes but is not limited to formate, acetate, propionate, glycolate, gluconate, lactate, oxalate, maleate, succinate, fumarate, tartrate, citrate, glutamate, aspartate, benzoate, methanesulfonate, p-toluenesulfonate, and salicylate. These salts can be prepared by methods known in this field. “Pharmaceutically acceptable base addition salt” includes but is not limited to salt derived from inorganic base such as sodium salt, potassium salt, calcium salt, and magnesium salt, etc.; and salt derived from organic base such as ammonium salt, triethylamine salt, lysine salt, arginine salt, etc. These salts can be prepared by methods known in this field.

In another preferred embodiment, R₁ is selected from substituted and unsubstituted C₅-C₈ aryl, substituted and unsubstituted 5-8 membered heteroaryl.

In another preferred embodiment, X is selected from —CO—, —SO₂—, and C₁-C₄ alkyl.

In another preferred embodiment, A is selected from C₁-C₃ alkyl, —CH₂—NH— [symbol] —CH═N—

[symbol]

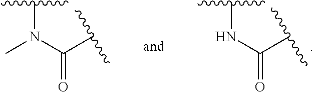

In another preferred embodiment, R₂ is selected from substituted and unsubstituted C₆-C₁₀ aryl, substituted and unsubstituted 4-10 membered heteroaryl, C₃-C₇ cycloalkyl. The C₆-C₁₀ aryl is optionally substituted by substituents selected from the group consisting of halogen, amino, trifluoromethyl, difluoromethyl, nitro, cyano, hydroxyl, —C(O)C₁-C₄ alkyl, C₁-C₄ alkyl, and C₁-C₄ haloalkyl.

As one embodiment, the spirocyclic compound is selected from any one of the following structures:

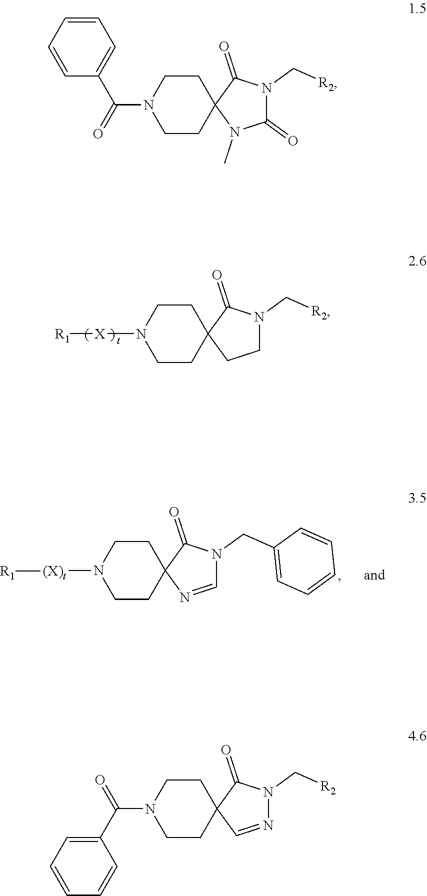

wherein,
X is —CO—, —SO₂—, C₁-C₄ alkyl; or
R₁ is selected from substituted and unsubstituted C₅-C₈ aryl, substituted and unsubstituted 5-8 membered heteroaryl; or
R₂ is selected from substituted and unsubstituted C₆-C₁₀ aryl, substituted and unsubstituted 4-10 membered heteroaryl, and C₃-C₇ cycloalkyl.

The second aspect of the present disclosure is to provide a preparation method for the compound described in the first aspect, wherein the preparation method comprises the following steps:

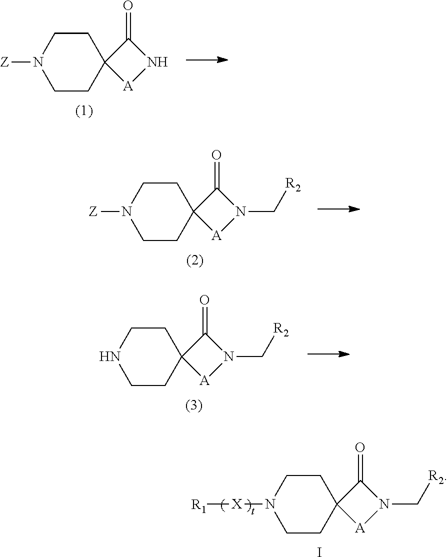

As one preferred embodiment, compound with a structure of formula 2.6 can be prepared through reaction route 2:

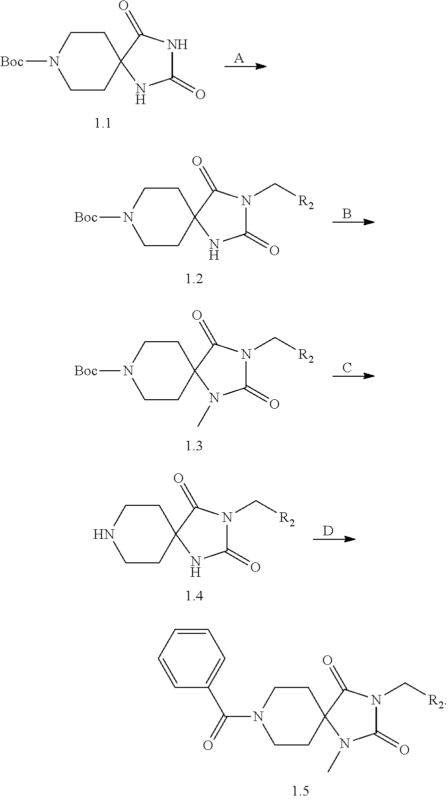

As one preferred embodiment, compound with a structure of formula 2.6 can be prepared through reaction route 2:

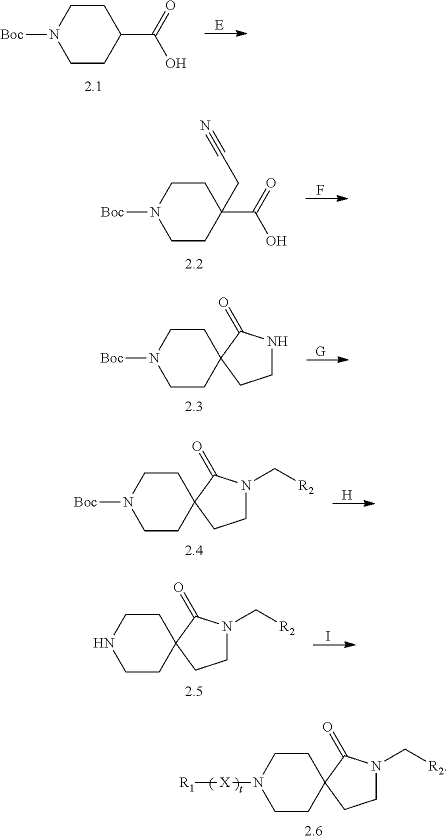

Step E: 1-(tert-butoxycarbonyl) piperidine-4-carboxylic acid (2.03 g, 8.30 mmol) was dissolved in 15 mL THE under a nitrogen atmosphere. A 2M THE solution of LDA (6.23 mL, 12.45 mmol) was slowly added at −78° C., and then stirred at −78° C. for 1 hour. Bromoacetonitrile (1.49 g, 12.45 mmol) was added dropwise slowly to the reaction mixture and stirred at −78° C. for 2 h, and then slowly rose to room temperature and reacted for 8 hours. After the reaction was completed, the reaction mixture was concentrated and added with ethyl acetate, the mixture was extracted with water 3 times, brine 1 time, and dried over anhydrous Na₂SO₄. Purification by silica gel chromatography afforded intermediate 1-(tert butoxycarbonyl)-4-(cyanomethyl) piperidine-4-carboxylic acid (formula 2.2, 972 mg, 41%). MS(ESI) m/z: 283.2 (M+1).

Step F: 1-(tert-butoxycarbonyl)-4-(cyanomethyl) piperidine-4-carboxylic acid (1.23 g, 4.39 mmol) and cobalt chloride hexahydrate (522.5 mg, 2.20 mmol) were mixed in methanol. Sodium borohydride (1.66 g, 43.92 mmol) was added in batches under the ice bath and stirred for 2 h, then the reaction was slowly raised to room temperature for 12 h and then heated to reflux reaction for 2 h. After the reaction was completed, the reaction mixture was partitioned between water and EtOAc. The ethyl acetate layers were washed with brine, dried over anhydrous Na₂SO₄. Purification by silica gel chromatography afforded tert-butyl 1-oxo-2,8-diazaspiro [4.5] decane-8-carboxylate (440 mg, 39%). ¹H NMR (300 MHz, CDCl₃) δ 5.66 (s, 1H), 4.15-3.85 (m, 2H), 3.35 (t, J=6.9 Hz, 2H), 3.10-2.88 (m, 2H), 2.07 (t, J=6.8 Hz, 2H), 1.94-1.78 (m, 2H), 1.46 (s, 9H).

As one preferred embodiment, compound with a structure of formula 3.5 can be prepared through reaction route 3.

### Reaction Route 3:

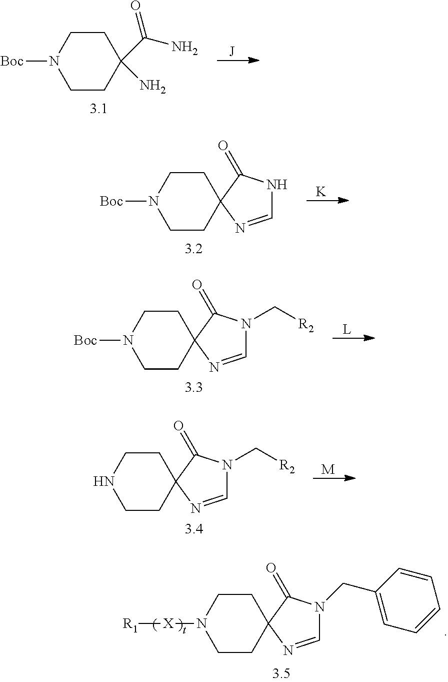

The preparation method of the key intermediate tert-butyl 4-oxo-1,3,8-triazaspiro [4.5]dec-1-ene-8-carboxylate (formula 3.2) is as follows:

Step J: The commercially available 4-amino-4-carbamylpiperidin-1-tert-butyl carboxylate (500 mg, 2.06 mmol) was suspended in toluene, and trimethyl orthoformate (654 mg, 6.17 mmol) and acetic acid (617 mg, 10.28 mmol) were added to the mixture and stirred at 90° C. for 12 h. When TLC showed that the reaction was completed, the reaction liquid was cooled to room temperature, the solvent was removed under reduced pressure, and the residue was extracted with ethyl acetate. The organic phase was washed with water, brine, and dried over anhydrous Na₂SO₄. Purification by silica gel chromatography to obtain 4-oxo-1,3,8-triazospiro [4.5] deca-1-en-8-carboxylic acid tert butyl ester (450 mg, 86%). ¹H NMR (300 MHz, CDCl3) δ 8.68 (s, 1H), 7.78 (s, 1H), 4.17-3.90 (m, 2H), 3.45-3.24 (m, 2H), 1.94-1.79 (m, 2H), 1.52-1.47 (m, 10H), 1.45-1.39 (m, 1H).

As one preferred embodiment, compound with a structure of formula 4.6 can be prepared through reaction route 4:

### Reaction Route 4:

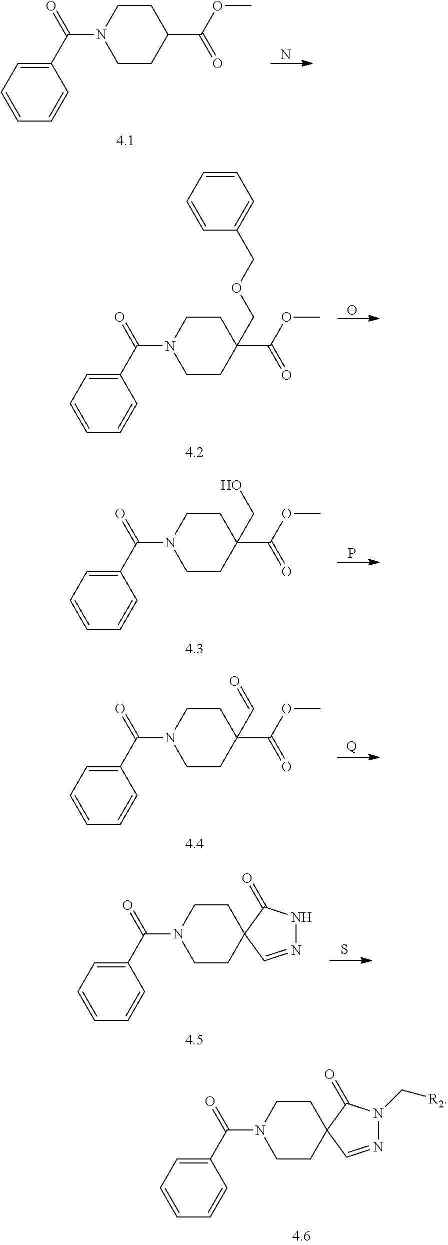

The preparation method of the key intermediate 8-benzoyl-2,3,8-triazaspiro [4.5] dec-3-en-1-one (formula 4.5) is as follows:

Step N: 1-Benzoylpiperidin-4-carboxylate methyl ester (2.4 g, 9.71 mmol) was dissolved in dry tetrahydrofuran, and a solution of LDA in tetrahydrofuran (2 M, 7.28 mL, 14.56 mmol) was added at −78° C. and stirred at −78° C. for 1 h, then chloromethyl benzyl ether (2.7 mL, 19.41 mmol) was added and stirred at −78° C. for 4 h. Then slowly rose to room temperature and continued for 6 h. After the reaction was completed, the reaction mixture was concentrated and added with ethyl acetate, the ethyl acetate layer was washed with saturated ammonium chloride solution, water, brine, dried over anhydrous Na₂SO₄, and then purified by column chromatography to obtain 1-benzoyl-4-((benzyloxy) methyl) piperidine-4-carboxylate methyl ester (2.15 g, 60%). ¹H NMR (400 MHz, CDCl₃) δ 7.44-7.28 (m, 8H), 7.28-7.27 (m, 1H), 7.26-7.24 (m, 1H), 4.49 (s, 2H), 4.41 (d, J=13.7 Hz, 1H), 3.74 (s, 3H), 3.58 (t, J=10.8 Hz, 1H), 3.54-3.38 (m, 2H), 3.22 (s, 1H), 3.04 (s, 1H), 2.27-2.09 (m, 2H), 1.65-1.54 (m, 1H), 1.51-1.38 (m, 1H).

Step O: 1-Benzoyl-4-((benzoxy) methyl) piperidine-4-carboxylate methyl ester (2.52 g, 6.86 mmol) was dissolved in methanol, palladium carbon (10%, 200 mg) was added, hydrogen was substituted for 3 times, and then stirred at 35° C. for 12 h. After the reaction was completed, filtered with diatomaceous earth. The filtrate was concentrated and dried to obtain 1-benzoyl-4-(hydroxymethyl) piperidine-4-carboxylate methyl ester, which was directly used in the next reaction. ¹H NMR (400 MHz, CDCl₃) δ 7.45-7.35 (m, 5H), 4.41-4.19 (m, 1H), 3.78 (s, 3H), 3.74-3.52 (m, 3H), 3.33-3.15 (m, 2H), 2.26-2.13 (m, 1H), 2.13-1.96 (m, 2H), 1.67-1.54 (m, 1H), 1.52-1.38 (m, 1H).

Step P: Oxaloyl chloride (615 μL, 7.21 mmol) was dissolved in dichloromethane, and DMSO (1.02 ml, 14.42 mmol) was added to dichloromethane solution under a nitrogen atmosphere, and stirred at −78° C. for 30 min. 1-Benzoyl-4-(hydroxymethyl) piperidine-4-carboxylate methyl ester (1 g, 3.61 mmol) was dissolved in dichloromethane (4 mL) and added to the reaction system at −78° C. After 2 hours, the DIPEA (3 mL, 18.03 mmol) was added for 1 h. After the reaction was completed, the mixture was washed with water, brine, and dried over anhydrous Na₂SO₄. Then purified by column chromatography to obtain 1-benzoyl-4-formylpiperidin-4-carboxylate methyl ester (970 mg, 98%). ¹H NMR (400 MHz, DMSO) δ 9.60 (s, 1H), 7.47-7.34 (m, 5H), 3.80 (s, 5H), 3.56-3.33 (m, 2H), 2.15-1.87 (m, 4H).

Step Q: 1-Benzoyl-4-formylpiperidin-4-carboxylate methyl ester (500 mg) was dissolved in methanol (5 mL), and hydrazine hydrate (98%, 300 μL) was added and stirred overnight at 40° C. After the reaction was completed, the reaction mixture was concentrated and dissolved in DCM. The organic phase was washed with water, and brine, and dried over anhydrous Na₂SO₄, and purified by column chromatography on silica gel to provide the 8-benzoyl-2,3,8-triazaspiro [4.5]dec-3-en-1-one (formula 4.5). ¹H NMR (400 MHz, DMSO) δ 11.35 (s, 1H), 7.81-7.73 (m, 1H), 7.50-7.31 (m, 5H), 4.34-4.08 (m, 1H), 3.77-3.57 (m, 1H), 3.52-3.36 (m, 2H), 1.73-1.45 (m, 4H).

In the above formulas, Z is selected from protecting groups such as Boc, Cbz, Fmoc, Trt, or Alloc, etc. R₁, X, A, and R₂ are defined as described in the first aspect.

In another preferred embodiment, raw material (1) reacts with a halomethyl-substituted C₆-C₁₀ aryl or a halomethyl-substituted 4-10 membered heteroaryl, and a C₃-C₇ cycloalkyl in an organic solvent with base as catalyst at room temperature to obtain intermediate (2) by conventional separation and purification.

In another preferred embodiment, intermediate (2) undergoes deprotection of the protective group Z by reacting with an acid in an organic solvent to obtain intermediate (3).

In another preferred embodiment, in an organic solvent, in the presence of a condensation agent and an organic base, intermediate (3) reacts with a halogen-substituted C₆-C₁₀ aryl, a halogen-substituted 4-10 membered heteroaryl, a carboxyl-substituted C₆-C₁₀ aryl, or a carboxyl-substituted 4-10 membered heteroaryl to prepare the compound shown in general Formula I.

In another preferred embodiment, in an organic solvent, in the presence of an organic base, intermediate (3) reacts with C₆-C₁₀ aryl substituted formyl chloride and 4-10 membered heteroaryl substituted formyl chloride to prepare the compound shown in general Formula I.

In another preferred embodiment, the above-mentioned organic solvents are selected from the group consisting of acetonitrile, dichloromethane, tetrahydrofuran, dioxane, DMF, or a mixture of two or more.

The third aspect of the present disclosure is to provide a pharmaceutical composition comprising the compound, the pharmaceutically acceptable salt, the stereoisomer, or the prodrug thereof as described in the first aspect, and a pharmaceutically acceptable carrier.

“Pharmaceutically acceptable carrier” refers to one or more compatible solid or liquid fillers or gel substances, which are suitable for human use and must possess sufficient purity and sufficiently low toxicity. “Compatibility” herein means that each component in the composition can be mixed with the active ingredient of the disclosure and with each other without significantly reducing the efficacy of the active ingredient. Some examples of pharmaceutically acceptable carrier include cellulose and its derivatives (such as sodium carboxymethyl cellulose, sodium ethyl cellulose, cellulose acetate, etc.), gelatin, talc, solid lubricants (such as stearic acid, magnesium stearate), calcium sulfate, vegetable oils (such as soybean oil, sesame oil, peanut oil, olive oil, etc.), polyols (such as propylene glycol, glycerol, mannitol, sorbitol, etc.), emulsifiers (such as Tween®), wetting agents (such as sodium dodecyl sulfate), colorants, flavorings, stabilizers, antioxidants, preservatives, pyrogen-free water, etc.

The solid dosage forms used for oral administration include capsules, tablets, pills, powders, and granules.

The liquid dosage forms for oral administration include pharmaceutically acceptable emulsions, solutions, suspensions, syrups, and tinctures. The compound of the present disclosure can be administered alone or in combination with other therapeutic drugs (such as antibiotics). When administering the compound or the pharmaceutical composition of the present disclosure, a safe and effective amount of the inventive compound is applied to the mammal (such as a human) requiring treatment, wherein the administered dose is considered an effective dosage by pharmaceutical standards. For a person weighing 60 kg, the daily dosage is usually 1-2000 mg, preferably 20-500 mg. Of course, the specific dosage should also consider factors such as the route of administration, the patient's health condition and other factors, all of which fall within the scope of expertise of a skilled physician.

The fourth aspect of the present disclosure is to provide a use of the compound as described in the first aspect in:
1) the preparation of a drug for inhibiting RIPK1 kinase activity; or
2) the preparation of a drug for preventing and/or treating RIPK1-related diseases.

The RIPK1-related diseases are tumor, ischemic stroke, rheumatoid arthritis, amyotrophic lateral sclerosis, multiple sclerosis, autoimmune disease, neurodegenerative disease, alcoholic steatohepatitis, non-alcoholic steatohepatitis, systemic inflammatory response syndrome, inflammatory bowel disease, or psoriasis.

### Beneficial Effect

The present disclosure is the first to synthesize a series of spirocyclic compounds, which are novel compounds and the preparation methods are also novel reaction routes. The spirocyclic compounds have the ability to inhibit RIPK1 kinase activity and can be used as a drug or prodrug for preventing and/or treating RIPK1 related diseases.

### BRIEF DESCRIPTION OF THE DRAWINGS

FIG. 1 is the results of TTC staining and slicing.

FIG. 2 is the statistical results of cerebral infarction volume; ** P<0.01, ***P<0.001, ****P<0.0001, compared to solvent control group by one-way analysis of variance.

FIG. 3 is the improved neurological impairment score; **P<0.01, ***P<0.001, ****P<0.0001, compared with the solvent control group by one-way analysis of variance.

### DETAILED DESCRIPTION OF THE PREFERRED EMBODIMENT

It should be understood that within the scope of the invention, the above technical features of the invention and the technical features specifically described below (embodiments) can be combined with each other to form a new or preferred technical solution. Each feature disclosed in the specification may be replaced by any alternative feature that provides the same, equal or similar purpose. Due to space limitations, I will not repeat them here.

### Definitions

“C₁-C₆” indicates that the group has 1-5 carbon atoms, C₆-C₁₀ indicates that the group has 6-10 carbon atoms, and so on. 4-10 membered means that the number of atoms in the ring is 3-8, and so on.

“Alkyl” refers to an unbranched or branched saturated hydrocarbon chain, including but not limited to methyl, ethyl, n-propyl, isopropyl, n-butyl, isobutyl, tert-butyl, sec-butyl, n-pentyl, etc. “Haloalkyl” refers to an unbranched or branched alkyl group as defined above, wherein one or more hydrogen atoms are replaced by a halogen, including but not limited to monofluoromethyl, monochloroethyl, difluoromethyl, dichloromethyl, dibromomethyl, trifluoromethyl, trichloromethyl, tribromomethyl, etc.

“Alkoxy” refers to “alkyl-O—”, the definition of the alkyl is described above. For example, “C₁-C₆ alkoxy” refers to an alkyl oxygen group containing 1-6 carbon atoms, including but not limited to methoxy, ethoxy, propoxy, butoxy, etc.

“Haloalkoxy” means —O-(haloalkyl), where haloalkyl is defined as described above.

“Heterocyclyl” refers to a saturated or partially unsaturated cyclic hydrocarbon group, with one or more ring heteroatoms independently selected from nitrogen, oxygen and sulfur, heterocyclyl may be a single ring or multiple rings (including spiral heterocyclyl, fused heterocyclyl, bridge heterocyclyl).

“Aryl” refers to an aromatic carbocyclic group having a single ring (e.g. monocyclic) or multiple rings including fused systems (A ring that shares pairs of adjacent carbon atoms), or polycyclic (a ring with adjacent pairs of carbon atoms) group, which has a conjugated R-electron system, such as phenyl, naphthyl.

“Heteroaryl” means a heteroaryl system consisting of 1-4 heteroatoms, including nitrogen, oxygen and sulfur, including but not limited to furanyl, thienyl, pyridinyl, pyrrolyl, N-alkyl pyrrolyl, pyrimidinyl, pyrazinyl, imidazolyl, tetrazolyl, etc.

Unless otherwise indicated, the term “substituted” used herein means any of the above groups and possible substituents include, but are not limited to: C₁-C₁₀ alkyl, C₂-C₁₀ alkenyl, C₂-C₁₀ alkynyl, C₃-C₂₀ cycloalkyl, C₃-C₂₀ cycloenyl, C₁-C₂₀ heterocyclyl, C₁-C₂₀ heterocyclic alkyl, C₁-C₁₀ alkoxy, aryl, heteraryl, heteraryl, amino, C₁-C₁₀ alkyl amino, C₁-C₂₀ dialkyl amino, aryl amino, diaryl amino group, cyano, nitro, acyl, thioacyl, acyloxy, carboxyl and carboxylate.

Unless otherwise defined, all professional and scientific terms used herein have the same meanings as those familiar to those skilled in the field. In addition, any methods and material is similar to or equal to the recorded content can be applied to the methods of the invention. The preferred implementation methods and materials described herein are for demonstration purposes only.

In the preparation examples and embodiments used, the NMR hydrogen spectrum was determined by Bruker AVANCE Neo 400 MHz or Bruker AVANCE 300 MHz, and the internal standard used was TMS. Low-resolution ESI mass spectrometry was determined by Finnigan LCQ-DECA mass spectrometer. Silica gel used in column chromatography is 100-200 mesh unless otherwise specified. The ratio of eluent is the volume ratio.

TABLE 1
List of compounds

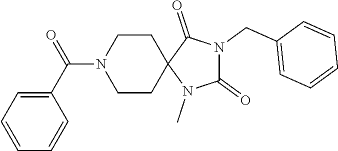 [SMILES: CN1C(=O)N(Cc2ccccc2)C(=O)C12CCN(C(=O)c1ccccc1)CC2]

2

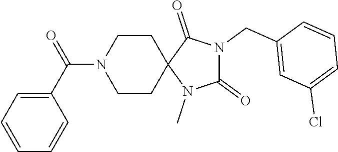 [SMILES: CN1C(=O)N(Cc2cccc(Cl)c2)C(=O)C12CCN(C(=O)c1ccccc1)CC2]

3

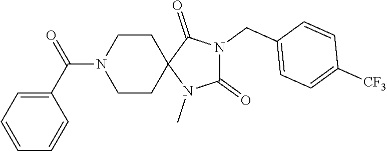 [SMILES: CN1C(=O)N(Cc2ccc(C(F)(F)F)cc2)C(=O)C12CCN(C(=O)c1ccccc1)CC2]

4

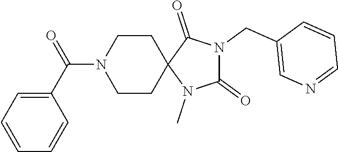 [SMILES: CN1C(=O)N(Cc2cccnc2)C(=O)C12CCN(C(=O)c1ccccc1)CC2]

5

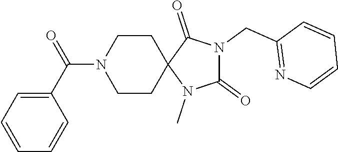 [SMILES: CN1C(=O)N(Cc2ccccn2)C(=O)C12CCN(C(=O)c1ccccc1)CC2]

6

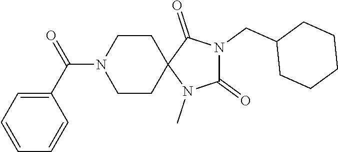 [SMILES: CN1C(=O)N(CC2CCCCC2)C(=O)C12CCN(C(=O)c1ccccc1)CC2]

7

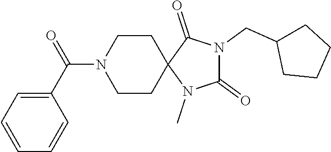 [SMILES: CN1C(=O)N(CC2CCCC2)C(=O)C12CCN(C(=O)c1ccccc1)CC2]

8

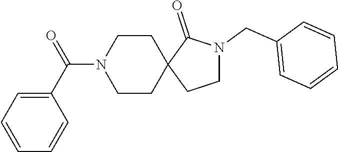 [SMILES: O=C(c1ccccc1)N1CCC2(CC1)CCN(Cc1ccccc1)C2=O]

9

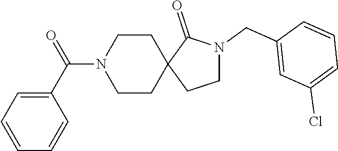 [SMILES: O=C(c1ccccc1)N1CCC2(CC1)CCN(Cc1cccc(Cl)c1)C2=O]

10

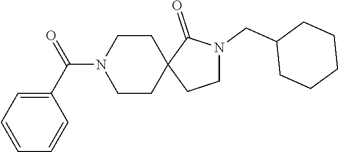 [SMILES: O=C(c1ccccc1)N1CCC2(CC1)CCN(CC1CCCCC1)C2=O]

11

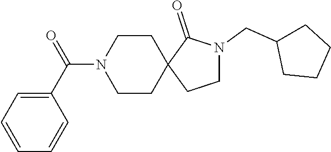 [SMILES: O=C(c1ccccc1)N1CCC2(CC1)CCN(CC1CCCC1)C2=O]

12

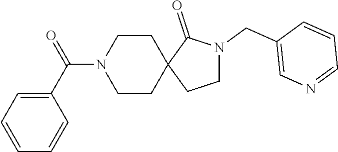 [SMILES: O=C(c1ccccc1)N1CCC2(CC1)CCN(Cc1cccnc1)C2=O]

13

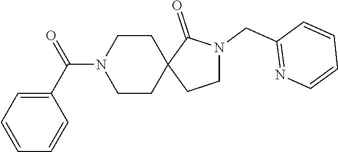 [SMILES: O=C(c1ccccc1)N1CCC2(CC1)CCN(Cc1ccccn1)C2=O]

14

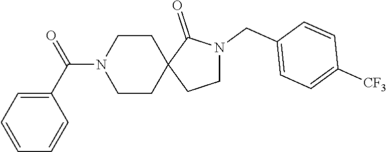 [SMILES: O=C(c1ccccc1)N1CCC2(CC1)CCN(Cc1ccc(C(F)(F)F)cc1)C2=O]

15

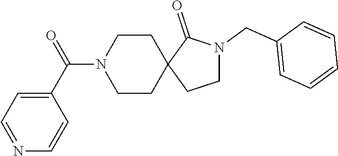 [SMILES: O=C(c1ccncc1)N1CCC2(CC1)CCN(Cc1ccccc1)C2=O]

16

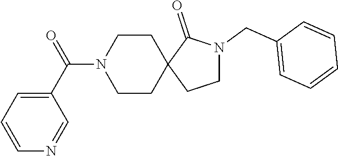 [SMILES: O=C(c1cccnc1)N1CCC2(CC1)CCN(Cc1ccccc1)C2=O]

17

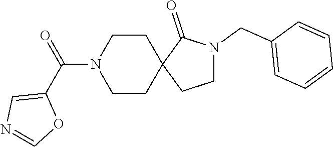 [SMILES: O=C(c1cnco1)N1CCC2(CC1)CCN(Cc1ccccc1)C2=O]

18

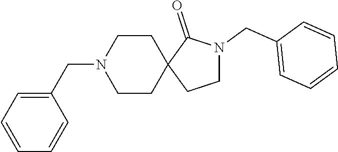 [SMILES: O=C1N(Cc2ccccc2)CCC12CCN(Cc1ccccc1)CC2]

19

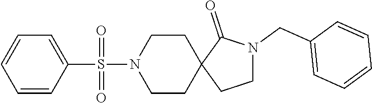 [SMILES: O=C1N(Cc2ccccc2)CCC12CCN(S(=O)(=O)c1ccccc1)CC2]

20

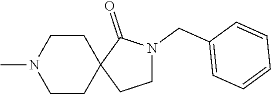 [SMILES: CN1CCC2(CC1)CCN(Cc1ccccc1)C2=O]

21

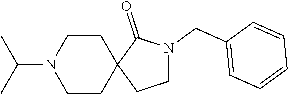 [SMILES: CC(C)N1CCC2(CCN(Cc3ccccc3)C2=O)CC1]

22

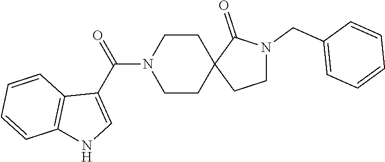 [SMILES: O=C(c1c[nH]c2ccccc12)N1CCC2(CC1)CCN(Cc1ccccc1)C2=O]

23

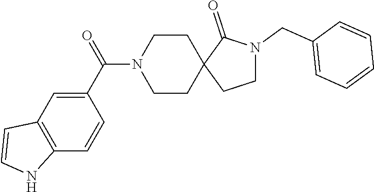 [SMILES: O=C(c1ccc2[nH]ccc2c1)N1CCC2(CC1)CCN(Cc1ccccc1)C2=O]

24

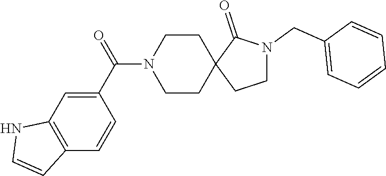 [SMILES: O=C(c1ccc2cc[nH]c2c1)N1CCC2(CC1)CCN(Cc1ccccc1)C2=O]

25

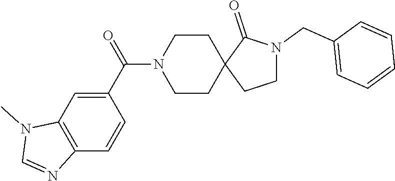 [SMILES: Cn1cnc2ccc(C(=O)N3CCC4(CC3)CCN(Cc3ccccc3)C4=O)cc21]

26

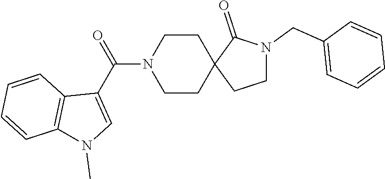 [SMILES: Cn1cc(C(=O)N2CCC3(CC2)CCN(Cc2ccccc2)C3=O)c2ccccc21]

27

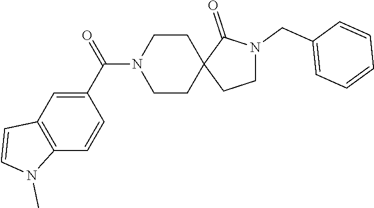 [SMILES: Cn1ccc2cc(C(=O)N3CCC4(CC3)CCN(Cc3ccccc3)C4=O)ccc21]

28

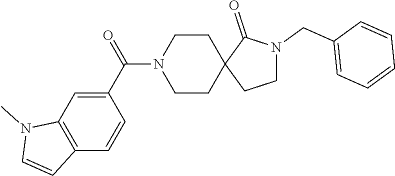 [SMILES: Cn1ccc2ccc(C(=O)N3CCC4(CC3)CCN(Cc3ccccc3)C4=O)cc21]

29

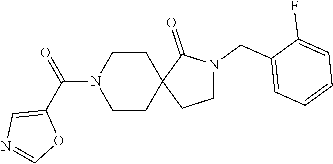 [SMILES: O=C(c1cnco1)N1CCC2(CC1)CCN(Cc1ccccc1F)C2=O]

30

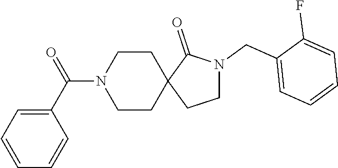 [SMILES: O=C(c1ccccc1)N1CCC2(CC1)CCN(Cc1ccccc1F)C2=O]

31

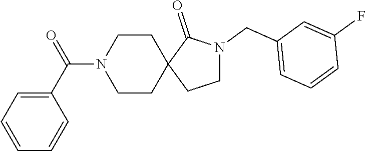 [SMILES: O=C(c1ccccc1)N1CCC2(CC1)CCN(Cc1cccc(F)c1)C2=O]

32

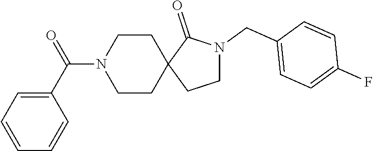 [SMILES: O=C(c1ccccc1)N1CCC2(CC1)CCN(Cc1ccc(F)cc1)C2=O]

33

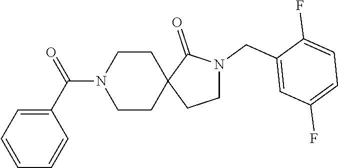 [SMILES: O=C(c1ccccc1)N1CCC2(CC1)CCN(Cc1cc(F)ccc1F)C2=O]

34

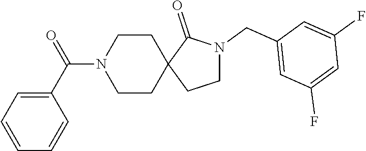 [SMILES: O=C(c1ccccc1)N1CCC2(CC1)CCN(Cc1cc(F)cc(F)c1)C2=O]

35

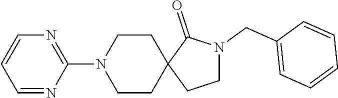 [SMILES: O=C1N(Cc2ccccc2)CCC12CCN(c1ncccn1)CC2]

36

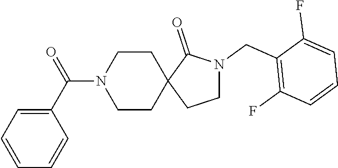 [SMILES: O=C(c1ccccc1)N1CCC2(CC1)CCN(Cc1c(F)cccc1F)C2=O]

37

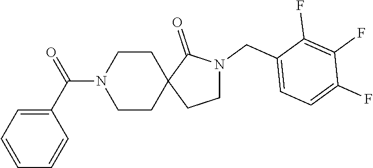 [SMILES: O=C(c1ccccc1)N1CCC2(CC1)CCN(Cc1ccc(F)c(F)c1F)C2=O]

38

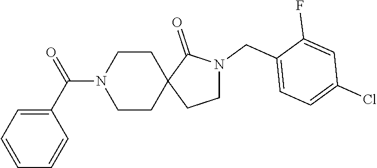 [SMILES: O=C(c1ccccc1)N1CCC2(CC1)CCN(Cc1ccc(Cl)cc1F)C2=O]

39

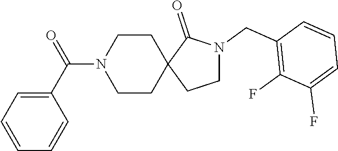 [SMILES: O=C(c1ccccc1)N1CCC2(CC1)CCN(Cc1cccc(F)c1F)C2=O]

40

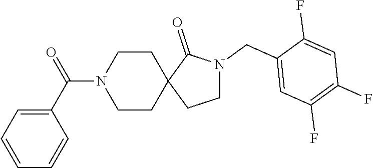 [SMILES: O=C(c1ccccc1)N1CCC2(CC1)CCN(Cc1cc(F)c(F)cc1F)C2=O]

41

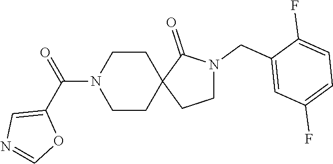 [SMILES: O=C(c1cnco1)N1CCC2(CC1)CCN(Cc1cc(F)ccc1F)C2=O]

42

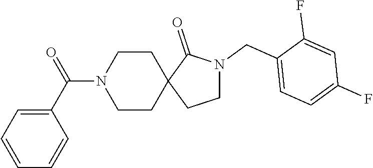 [SMILES: O=C(c1ccccc1)N1CCC2(CC1)CCN(Cc1ccc(F)cc1F)C2=O]

43

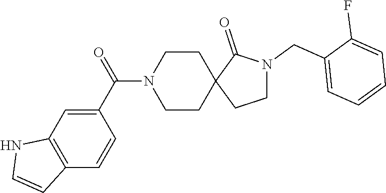 [SMILES: O=C(c1ccc2cc[nH]c2c1)N1CCC2(CC1)CCN(Cc1ccccc1F)C2=O]

44

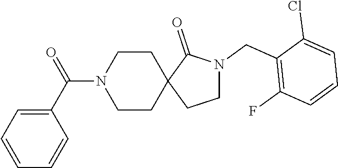 [SMILES: O=C(c1ccccc1)N1CCC2(CC1)CCN(Cc1c(F)cccc1Cl)C2=O]

45

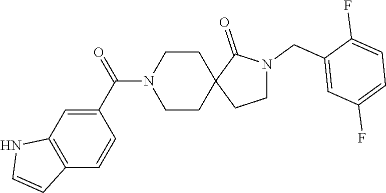 [SMILES: O=C(c1ccc2cc[nH]c2c1)N1CCC2(CC1)CCN(Cc1cc(F)ccc1F)C2=O]

46

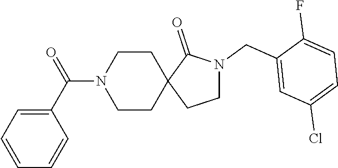 [SMILES: O=C(c1ccccc1)N1CCC2(CC1)CCN(Cc1cc(Cl)ccc1F)C2=O]

47

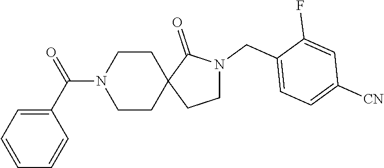 [SMILES: N#Cc1ccc(CN2CCC3(CCN(C(=O)c4ccccc4)CC3)C2=O)c(F)c1]

48

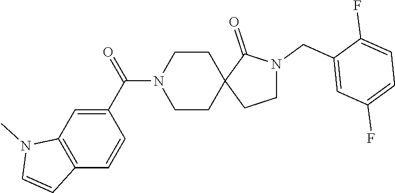 [SMILES: Cn1ccc2ccc(C(=O)N3CCC4(CC3)CCN(Cc3cc(F)ccc3F)C4=O)cc21]

49

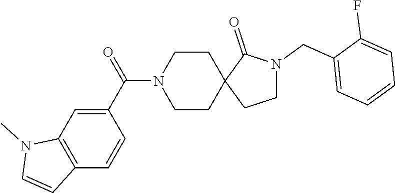 [SMILES: Cn1ccc2ccc(C(=O)N3CCC4(CC3)CCN(Cc3ccccc3F)C4=O)cc21]

50

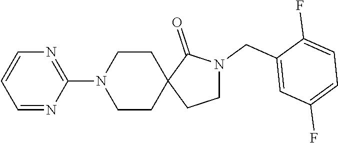 [SMILES: O=C1N(Cc2cc(F)ccc2F)CCC12CCN(c1ncccn1)CC2]

51

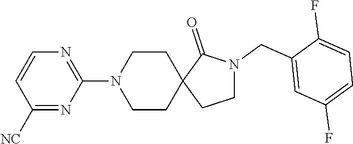 [SMILES: N#Cc1ccnc(N2CCC3(CCN(Cc4cc(F)ccc4F)C3=O)CC2)n1]

52

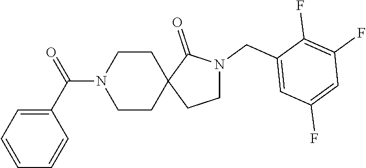 [SMILES: O=C(c1ccccc1)N1CCC2(CC1)CCN(Cc1cc(F)cc(F)c1F)C2=O]

53

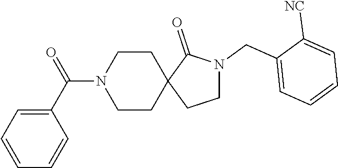 [SMILES: N#Cc1ccccc1CN1CCC2(CCN(C(=O)c3ccccc3)CC2)C1=O]

54

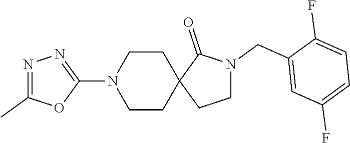 [SMILES: Cc1nnc(N2CCC3(CCN(Cc4cc(F)ccc4F)C3=O)CC2)o1]

55

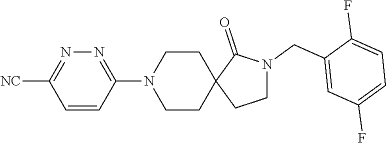 [SMILES: N#Cc1ccc(N2CCC3(CCN(Cc4cc(F)ccc4F)C3=O)CC2)nn1]

56

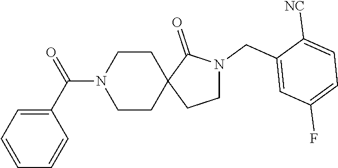 [SMILES: N#Cc1ccc(F)cc1CN1CCC2(CCN(C(=O)c3ccccc3)CC2)C1=O]

57

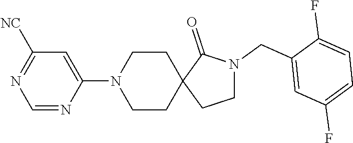 [SMILES: N#Cc1cc(N2CCC3(CCN(Cc4cc(F)ccc4F)C3=O)CC2)ncn1]

58

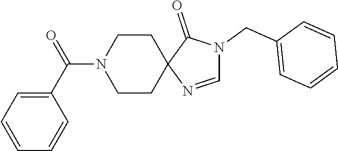 [SMILES: O=C(c1ccccc1)N1CCC2(CC1)N=CN(Cc1ccccc1)C2=O]

59

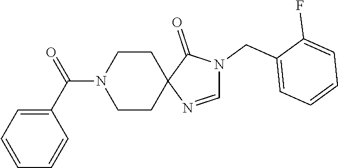 [SMILES: O=C(c1ccccc1)N1CCC2(CC1)N=CN(Cc1ccccc1F)C2=O]

60

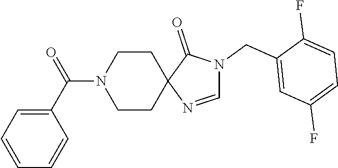 [SMILES: O=C(c1ccccc1)N1CCC2(CC1)N=CN(Cc1cc(F)ccc1F)C2=O]

61

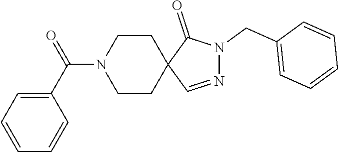 [SMILES: O=C(c1ccccc1)N1CCC2(C=NN(Cc3ccccc3)C2=O)CC1]

62

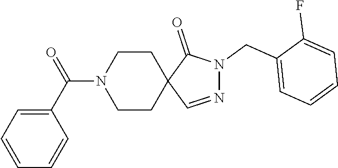 [SMILES: O=C(c1ccccc1)N1CCC2(C=NN(Cc3ccccc3F)C2=O)CC1]

63

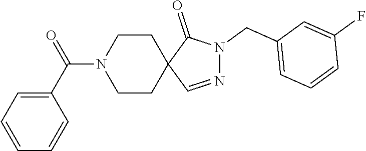 [SMILES: O=C(c1ccccc1)N1CCC2(C=NN(Cc3cccc(F)c3)C2=O)CC1]

64

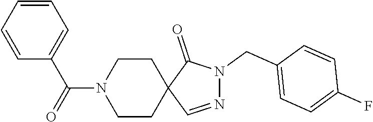 [SMILES: O=C(c1ccccc1)N1CCC2(C=NN(Cc3ccc(F)cc3)C2=O)CC1]

65

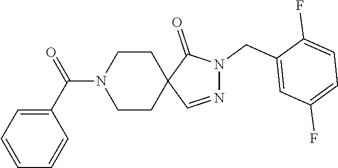 [SMILES: O=C(c1ccccc1)N1CCC2(C=NN(Cc3cc(F)ccc3F)C2=O)CC1]

66

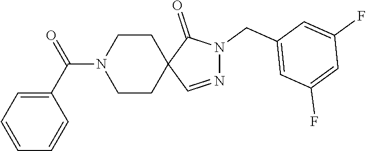 [SMILES: O=C(c1ccccc1)N1CCC2(C=NN(Cc3cc(F)cc(F)c3)C2=O)CC1]

67

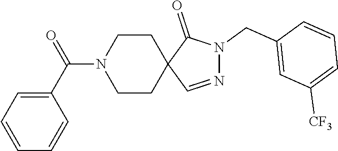 [SMILES: O=C(c1ccccc1)N1CCC2(C=NN(Cc3cccc(C(F)(F)F)c3)C2=O)CC1]

68

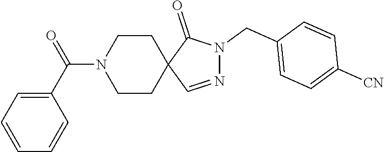 [SMILES: N#Cc1ccc(CN2N=CC3(CCN(C(=O)c4ccccc4)CC3)C2=O)cc1]

69

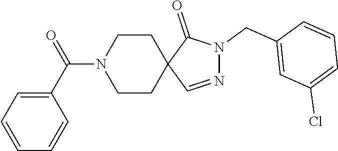 [SMILES: O=C(c1ccccc1)N1CCC2(C=NN(Cc3cccc(Cl)c3)C2=O)CC1]

70

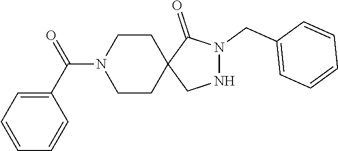 [SMILES: O=C(c1ccccc1)N1CCC2(CC1)CNN(Cc1ccccc1)C2=O]

71

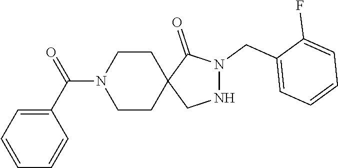 [SMILES: O=C(c1ccccc1)N1CCC2(CC1)CNN(Cc1ccccc1F)C2=O]

72

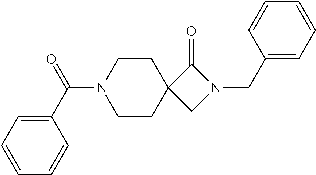 [SMILES: O=C(c1ccccc1)N1CCC2(CC1)CN(Cc1ccccc1)C2=O]

73

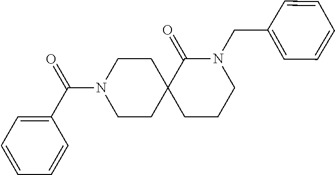 [SMILES: O=C(c1ccccc1)N1CCC2(CCCN(Cc3ccccc3)C2=O)CC1]

74

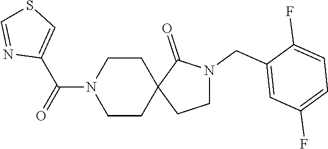 [SMILES: O=C(c1cscn1)N1CCC2(CC1)CCN(Cc1cc(F)ccc1F)C2=O]

75

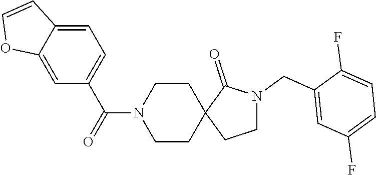 [SMILES: O=C(c1ccc2ccoc2c1)N1CCC2(CC1)CCN(Cc1cc(F)ccc1F)C2=O]

76

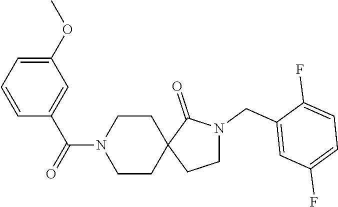 [SMILES: COc1cccc(C(=O)N2CCC3(CC2)CCN(Cc2cc(F)ccc2F)C3=O)c1]

77

Some methods for preparing compounds of the disclosure are described in the reaction routes and embodiments below. Starting materials and intermediates may be purchased, prepared using known methods, or otherwise specified. The following reaction routes also describe the commonly used pathways for preparing compounds of formula I. In some cases, the sequence of steps in the reaction routes can be changed to promote the reactions or avoid the generation of by-products. Substituents such as “A, R₁, R₂, X” in the reaction routes and their definitions are equivalent to the substituents defined by formula I at the same position in the structures.

### Example 1

Compounds with a structure of formula 1.5 can be prepared through reaction route 1.

### Reaction Route 1

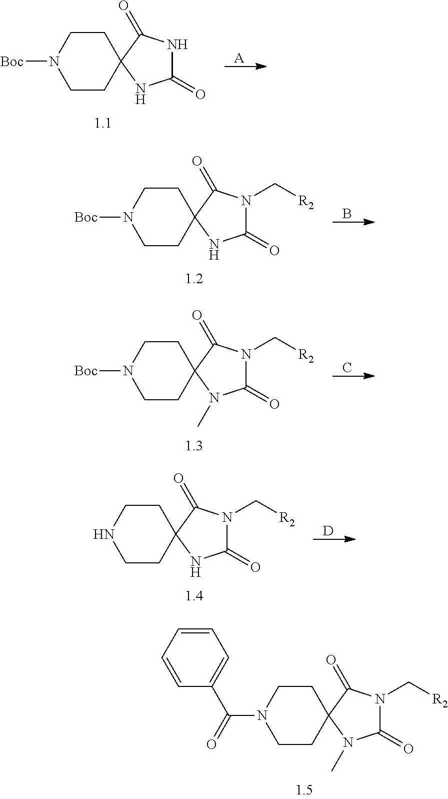

The following compounds were synthesized according to reaction route 1:

### Compound 2: 8-benzoyl-3-benzyl-1-methyl-1,3,8-triazaspiro [4.5] decane-2,4-dione

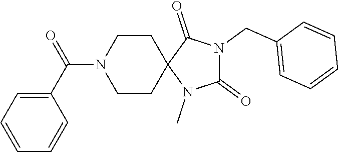 [SMILES: CN1C(=O)N(Cc2ccccc2)C(=O)C12CCN(C(=O)c1ccccc1)CC2]

Step A: Tert-butyl 2,4-dioxo-1,3,8-triazaspiro[4.5]decane-8-carboxylate (300 mg, 1.11 mmol), potassium carbonate (230 mg, 1.67 mmol) and benzyl bromide (210 mg, 1.23 mmol) were mixed in 5 mL DMF stirred at room temperature for 3 hours. TLC showed that the reaction was completed, the reaction mixture was partitioned between water (30 ml) and EtOAc (10 mL×3). The organic layers were washed with brine, dried over anhydrous Na₂SO₄, and then concentrated under reduced pressure to afford the crude product. Purification of the crude material by silica gel chromatography. Obtain the intermediate 3-benzyl-2-benzyl 2-,4-dioxa-1,3,8-triazaspiro [4.5] decane-8-carboxylic acid tert butyl ester (333 mg, 83%). ¹H NMR (300 MHz, Chloroform-d) δ 7.52 (d, J=5.2 Hz, 1H), 7.22 (d, J=14.6 Hz, 5H), 4.56 (d, J=2.3 Hz, 2H), 3.93 (d, J=13.8 Hz, 2H), 3.08 (t, J=12.2 Hz, 2H), 1.91 (ddd, J=14.5, 10.4, 4.3 Hz, 2H), 1.50 (d, J=13.9 Hz, 2H), 1.40 (d, J=2.5 Hz, 9H).

Step B: The intermediate 3-benzyl-2-benzyl 2-,4-dioxazo-1,3,8-triazaspiro [4.5]decane-8-carboxylic acid tert butyl ester (150 mg, 0.42 mmol) was dissolved in THE (3 mL), sodium hydroxide (25 mg, 0.63 mmol) and iodomethane (65 mg, 0.46 mmol) were added and stirred at room temperature for 3 h. When TLC showed that the reaction was completed, the reaction mixture was concentrated and added with ethyl acetate (123 mg, 79%), the mixture was extracted with water, brine, dried over Na₂SO₄, and then concentrated under reduced pressure to afford the crude product. Purification of the crude material by silica gel chromatography afforded intermediate 3-benzyl-1-methyl-1-methyl-2,4-dioxa-1,3,8-triazaspiro [4.5] decane-8-carboxylic acid tert butyl ester. MS(ESI) m/z: 374.2 (M+1).

Step C: The intermediate 3-benzyl-1-methyl-1-methyl,2,4-dioxa-1,3,8-triazaspiro [4.5]decane-8-carboxylic acid tert butyl ester (100 mg) was dissolved in dichloromethane (4 mL), and trifluoroacetic acid (400 μL) was added for 2 h at room temperature. When TLC showed that the reaction was completed, the saturated sodium bicarbonate solution was added until no bubbles were formed, and the reaction mixture was partitioned between water and EtOAc. The combined organic phase was dried with anhydrous Na₂SO₄ and then concentrated in vacuo to afford the crude product of the intermediate 3-benzyl-1-methyl-1,3,8-triazaspiro [4.5] decane-2,4-dione, which was directly used in the next reaction.

Step D: The crude product of the intermediate 3-benzyl-1-methyl-1,3,8-triazaspiro[4.5]decane 2,4-dione was dissolved in dichloromethane, added triethylamine, and then added benzoyl chloride under an ice bath, and slowly rose to room temperature, stirred for 1 hour. After TLC showed that the reaction was completed, the reaction mixture was washed with saturated ammonium chloride solution, washed with water 3 times, brine 1 time, and dried over anhydrous Na₂SO₄, and then concentrated under reduced pressure to afford the crude product. Purification of the crude material by silica gel chromatography. Vacuum distillation and purification by column chromatography afforded compound 2. ¹H NMR (300 MHz, CDCl3) δ 7.48-7.39 (m, 5H), 7.39-7.28 (m, 5H), 4.81-4.69 (m, 1H), 4.66 (s, 2H), 3.96-3.69 (m, 2H), 3.66-3.46 (m, 1H), 2.85 (s, 3H), 2.10-1.90 (m, 1H), 1.82-1.68 (m, 2H), 1.60-1.48 (m, 1H). MS(ESI)m/z: 378.2 (M+H)+.

### Compound 3: 8-benzoyl-3-(3-chlorobenzyl)-1-methyl-1,3,8-triazaspiro[4.5]decane-2,4-dione

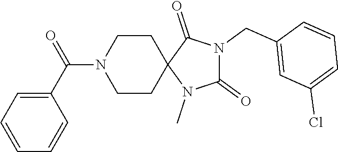 [SMILES: CN1C(=O)N(Cc2cccc(Cl)c2)C(=O)C12CCN(C(=O)c1ccccc1)CC2]

Similar to the steps described in compound 2,8-benzoyl-3-(3-chlorobenzyl)-1-methyl-1,3,8-triazaspiro[4.5]decane-2,4-dione was synthesized from 2,4-dioxa-1,3,8-triazaspiro [4.5] decane-8-carboxylic acid tert butyl ester through a three steps reaction with the corresponding chemical reagents. ¹H NMR (300 MHz, CDCl3) δ 7.49-7.38 (m, 5H), 7.38 (s, 1H), 7.29-7.19 (m, 3H), 4.84-4.68 (m, 1H), 4.62 (s, 2H), 3.95-3.69 (m, 2H), 3.67-3.45 (m, 1H), 2.86 (s, 3H), 2.13-1.92 (m, 1H), 1.86-1.66 (m, 2H), 1.63-1.47 (m, 1H). MS(ESI)m/z: 412.1 (M+H)+.

### Compound 4: 8-benzoyl-1-methyl-3-(4-(trifluoromethyl)benzyl)-1,3,8-triazaspiro decane-2,4-dione

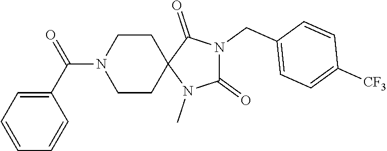 [SMILES: CN1C(=O)N(Cc2ccc(C(F)(F)F)cc2)C(=O)C12CCN(C(=O)c1ccccc1)CC2]

Similar to the steps described in compound 2,8-benzoyl-1-methyl-3-(4-(trifluoromethyl)benzyl)-1,3,8-triazaspiro [4.5] decane-2,4-dione was synthesized from 2,4-dioxa-1,3,8-triazaspiro [4.5] decane-8-carboxylic acid tert butyl ester through a three steps reaction with the corresponding chemical reagents. ¹H NMR (300 MHz, CDCl₃) δ 7.58 (d, J=8.1 Hz, 2H), 7.48 (d, J=8.1 Hz, 2H), 7.45-7.39 (m, 5H), 4.84-4.65 (m, 3H), 3.90-3.71 (m, 2H), 3.64-3.44 (m, 1H), 2.87 (s, 3H), 2.13-1.94 (m, 1H), 1.85-1.66 (m, 2H), 1.60-1.48 (m, 1H). MS(ESI)m/z: 446.2 (M+H)+.

### Compound 5: 8-benzoyl-1-methyl-3-(pyridin-3-ylmethyl)-1,3,8-triazaspiro [4.5]decane-2,4-dione

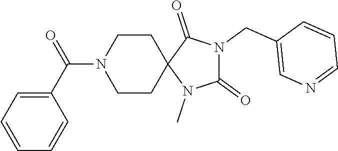 [SMILES: CN1C(=O)N(Cc2cccnc2)C(=O)C12CCN(C(=O)c1ccccc1)CC2]

Similar to the steps described in compound 2,8-benzoyl-1-methyl-3-(pyridin-3-ylmethyl)-1,3,8-triazaspiro [4.5] decane-2,4-dione was synthesized from 2,4-dioxa-1,3,8-triazaspiro [4.5] decane-8-carboxylic acid tert butyl ester through a three steps reaction with the corresponding chemical reagents. ¹H NMR (300 MHz, CDCl₃) δ 8.65 (d, J=2.3 Hz, 1H), 8.55 (dd, J=4.9, 1.7 Hz, 1H), 7.72 (dt, J=7.9, 2.1 Hz, 1H), 7.43 (s, 5H), 7.32-7.22 (m, 2H), 4.84-4.70 (m, 1H), 4.68 (s, 2H), 3.96-3.68 (m, 2H), 3.65-3.45 (m, 1H), 2.86 (s, 3H), 2.12-1.93 (m, 1H), 1.85-1.67 (m, 3H), 1.63-1.49 (m, 1H). MS(ESI)m/z: 379.2 (M+H)+.

### Compound 6: 8-benzoyl-1-methyl-3-(pyridin-2-ylmethyl)-1,3,8-triazaspiro [4.5]decane-2,4-dione

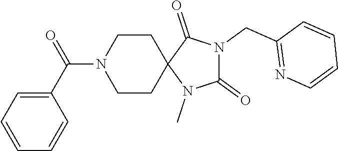 [SMILES: CN1C(=O)N(Cc2ccccn2)C(=O)C12CCN(C(=O)c1ccccc1)CC2]

Similar to the steps described in compound 2,8-benzoyl-1-methyl-3-(pyridin-2-ylmethyl)-1,3,8-triazaspiro [4.5] decane-2,4-dione was synthesized from 2,4-dioxa-1,3,8-triazaspiro [4.5] decane-8-carboxylic acid tert butyl ester through a three steps reaction with the corresponding chemical reagents. ¹H NMR (300 MHz, CDCl₃) δ 8.50 (dt, J=4.7, 1.5 Hz, 1H), 7.64 (td, J=7.7, 1.8 Hz, 1H), 7.49-7.39 (m, 5H), 7.23 (d, J=7.8 Hz, 1H), 7.17 (ddd, J=7.6, 4.9, 1.1 Hz, 1H), 4.83 (s, 2H), 4.81-4.70 (m, 1H), 3.97-3.72 (m, 2H), 3.65-3.49 (m, 1H), 2.90 (s, 3H), 2.21-1.98 (m, 1H), 1.97-1.78 (m, 2H), 1.77-1.63 (m, 1H). MS(ESI)m/z: 379.2 (M+H)+.

### Compound 7: 8-benzoyl-3-(cyclohexylmethyl)-1-methyl-1,3,8-triazaspiro [4.5]decane-2,4-dione

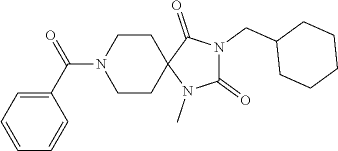 [SMILES: CN1C(=O)N(CC2CCCCC2)C(=O)C12CCN(C(=O)c1ccccc1)CC2]

Similar to the steps described in compound 2,8-benzoyl-3-(cyclohexylmethyl)-1-methyl-1,3,8-triazaspiro[4.5]decane-2,4-dione was synthesized from 2,4-dioxa-1,3,8-triazaspiro [4.5] decane-8-carboxylic acid tert butyl ester through a three steps reaction with the corresponding chemical reagents. ¹H NMR (300 MHz, CDCl₃) δ 7.59-7.32 (m, 5H), 4.84-4.64 (m, 1H), 4.01-3.70 (m, 2H), 3.69-3.48 (m, 1H), 3.34 (d, J=7.3 Hz, 2H), 2.86 (s, 3H), 2.15-1.92 (m, 1H), 1.88-1.47 (m, 9H), 1.34-1.08 (m, 4H), 1.07-0.75 (m, 2H). MS(ESI)m/z: 384.2 (M+H)+.

### Compound 8: 8-benzoyl-3-(cyclopentylmethyl)-1-methyl-1,3,8-triazaspiro [4.5]decane-2,4-dione

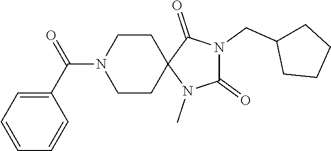 [SMILES: CN1C(=O)N(CC2CCCC2)C(=O)C12CCN(C(=O)c1ccccc1)CC2]

Similar to the steps described in compound 2,8-benzoyl-3-(cyclopentylmethyl)-1-methyl-1,3,8-triazaspiro[4.5]decane-2,4-dione was synthesized from 2,4-dioxa-1,3,8-triazaspiro [4.5] decane-8-carboxylic acid tert butyl ester through a three steps reaction with the corresponding chemical reagents. ¹H NMR (300 MHz, CDCl₃) δ 7.46-7.39 (m, 5H), 4.86-4.67 (m, 1H), 3.96-3.72 (m, 2H), 3.70-3.50 (m, 1H), 3.45 (d, J=7.8 Hz, 2H), 2.86 (s, 3H), 2.13-1.94 (m, 1H), 1.89-1.46 (m, 9H), 1.36-1.13 (m, 3H). MS(ESI)m/z: 370.2 (M+H)+.

### Example 2

Compounds with a structure of formula 2.6 can be prepared through reaction route 2.

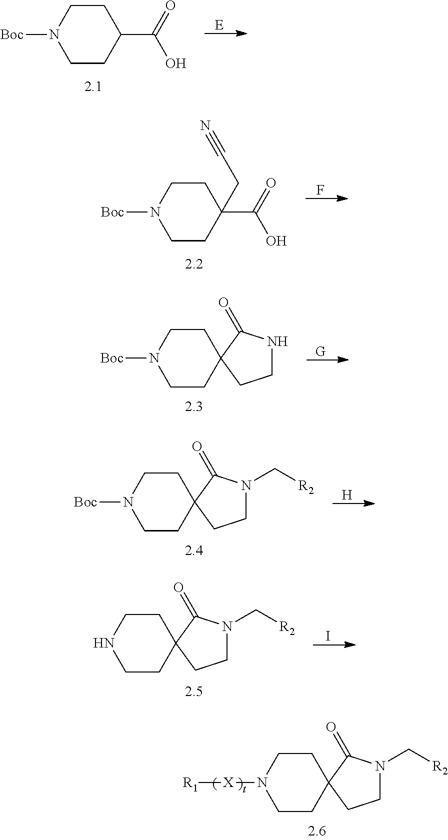

The preparation method of the key intermediate tert-butyl 1-oxo-2,8-diazaspiro [4.5]decane-8-carboxylate (formula 2.3) is as follows:

Step E: 1-(Tert-butoxycarbonyl) piperidine-4-carboxylic acid (2.03 g, 8.30 mmol) was dissolved in 15 mL THE under a nitrogen atmosphere. A 2M THE solution of LDA (6.23 mL, 12.45 mmol) was slowly added at −78° C., and then stirred at −78° C. for 1 hour. Bromoacetonitrile (1.49 g, 12.45 mmol) was added dropwise slowly to the reaction mixture and stirred at −78° C. for 2 h, and then slowly rose to room temperature for 8 hours. After the reaction was completed, the reaction mixture was concentrated and added with ethyl acetate, the mixture was extracted with water 3 times, brine 1 time, and dried over anhydrous Na₂SO₄. Purification by silica gel chromatography afforded intermediate 1-(tert butoxycarbonyl)-4-(cyanomethyl) piperidine-4-carboxylic acid (formula 2.2, 972 mg, 41%). MS(ESI) m/z: 283.2 (M+1).

Step F: 1-(tert-butoxycarbonyl)-4-(cyanomethyl) piperidine-4-carboxylic acid (1.23 g, 4.39 mmol) and cobalt chloride hexahydrate (522.5 mg, 2.20 mmol) were mixed in methanol. Sodium borohydride (1.66 g, 43.92 mmol) was added in batches under the ice bath and stirred for 2 h, then the reaction was slowly raised to room temperature for 12 h and then heated to reflux reaction for 2 h. At the end of the reaction, the reaction mixture was partitioned between water and EtOAc. The ethyl acetate layers were washed with brine, dried over anhydrous Na₂SO₄. Purification of the crude material by silica gel chromatography afforded tert-butyl 1-oxo-2,8-diazaspiro [4.5]decane-8-carboxylate (440 mg, 39%). ¹H NMR (300 MHz, CDCl₃) δ5.66 (s, 1H), 4.15-3.85 (m, 2H), 3.35 (t, J=6.9 Hz, 2H), 3.10-2.88 (m, 2H), 2.07 (t, J=6.8 Hz, 2H), 1.94-1.78 (m, 2H), 1.46 (s, 9H).

### Compound 9: 8-benzoyl-2-benzyl-2,8-diazaspiro[4.5]decan-1-one

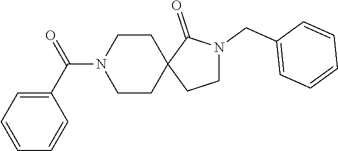 [SMILES: O=C(c1ccccc1)N1CCC2(CC1)CCN(Cc1ccccc1)C2=O]

Step G: Tert-butyl 1-oxo-2,8-diazaspiro [4.5]decane-8-carboxylate (1 g, 3.93 mmol) was dissolved in DMF (10 mL), then sodium hydroxide (189 mg, 4.72 mmol) and benzyl bromide (807 mg, 4.72 mmol) were added and stirred at room temperature for 4 h. After the reaction is completed, the reaction mixture was partitioned between water and EtOAc. The ethyl acetate layers were washed with brine, and dried over anhydrous Na₂SO₄. Purification of the crude material by silica gel chromatography afforded 2-benzyl-1-oxo-2,8-diazspiro [4.5]decane-8-carboxylic acid tert butyl ester (1.29 g, 95%). ¹H NMR (300 MHz, CDCl₃) δ 7.32 (q, J=11.1, 10.0 Hz, 3H), 7.25-7.16 (m, 2H), 4.46 (s, 2H), 4.12-3.92 (m, 2H), 3.18 (t, J=6.9 Hz, 2H), 3.04-2.90 (m, 2H), 2.00-1.83 (m, 4H), 1.47 (s, 9H), 1.44-1.34 (m, 2H).

Step H: 2-Benzyl-1-oxo-2,8-diazspiro [4.5]decane-8-carboxylic acid tert butyl ester was dissolved in dichloromethane (5 mL), and trifluoroacetic acid (500 μL) was added for 2 h at room temperature. When TLC showed that the reaction was completed, the saturated sodium bicarbonate solution was added until no bubbles were formed, and the reaction mixture was partitioned between DCM and EtOAc. The combined organic phase was dried with anhydrous Na₂SO₄ and then concentrated under reduced pressure to afford the crude product of the intermediate 2-benzyl-2,8-diazonium [4.5]decan-1-one, which was directly used in the next reaction.

Step I: 2-Benzyl-2,8-diazospirine [4.5]decan-1-one (47 mg, 0.19 mmol) was dissolved in dichloromethane and then DIPEA (32 μL, 0.23 mmol) was added; Benzoyl chloride (27 μL, 0.23 mmol) was added to the ice bath and slowly rose to room temperature stirred for 1 h. After TLC showed that the reaction was completed, the reaction mixture was washed with saturated ammonium chloride solution, washed with water 3 times, brine 1 time, dried over Na₂SO₄, and then concentrated under reduced pressure to afford the crude product. Purification of the crude material by silica gel chromatography. Vacuum distillation and purification by column chromatography afforded 8-benzoyl-2-benzyl-2,8-diazaspiro[4.5]decan-1-one (57 mg, 85%). ¹H NMR (300 MHz, CDCl₃) δ 7.41 (s, 5H), 7.37-7.27 (m, 3H), 7.21 (d, J=7.2 Hz, 2H), 4.54-4.36 (m, 3H), 3.91-3.71 (m, 1H), 3.36-3.05 (m, 4H), 2.09-1.85 (m, 4H), 1.61-1.35 (m, 2H). MS(ESI)m/z: 349.2 (M+H)⁺.

### Compound 10: 8-benzoyl-2-(3-chlorobenzyl)-2,8-diazaspiro[4.5]decan-1-one

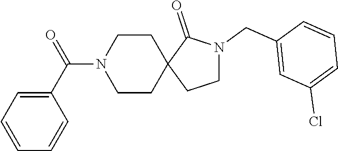 [SMILES: O=C(c1ccccc1)N1CCC2(CC1)CCN(Cc1cccc(Cl)c1)C2=O]

Similar to the steps described in compound 9, 8-benzoyl-2-(3-chlorobenzyl)-2,8-diazaspiro[4.5]decan-1-one was synthesized from tert-butyl 1-oxo-2,8-diazaspiro [4.5]decane-8-carboxylate (formula 2.3) through a three-step reaction with the corresponding chemical reagents. ¹H NMR (300 MHz, CDCl₃) δ 7.46-7.37 (m, 5H), 7.26 (d, J=4.6 Hz, 2H), 7.19 (s, 1H), 7.10 (t, J=4.4 Hz, 1H), 4.55-4.35 (m, 3H), 3.97-3.69 (m, 1H), 3.36-3.12 (m, 4H), 2.10-1.86 (m, 4H), 1.66-1.41 (m, 2H). MS(ESI)m/z: 383.1 (M+H)+.

### Compound 11: 8-benzoyl-2-(cyclohexylmethyl)-2,8-diazaspiro[4.5]decan-1-one

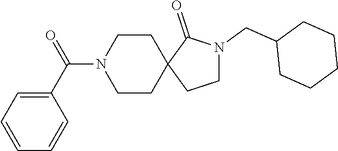 [SMILES: O=C(c1ccccc1)N1CCC2(CC1)CCN(CC1CCCCC1)C2=O]

Similar to the steps described in compound 9, 8-benzoyl-2-(cyclohexylmethyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (300 MHz, CDCl₃) δ 7.44-7.37 (m, 5H), 4.51-4.33 (m, 1H), 3.86-3.71 (m, 1H), 3.37-3.14 (m, 4H), 3.11 (d, J=7.1 Hz, 2H), 2.10-1.85 (m, 4H), 1.79-1.50 (m, 8H), 1.29-1.11 (m, 4H), 1.02-0.86 (m, 2H). MS(ESI)m/z: 355.3 (M+H)⁺.

### Compound 12: 8-benzoyl-2-(cyclopentylmethyl)-2,8-diazaspiro[4.5]decan-1-one

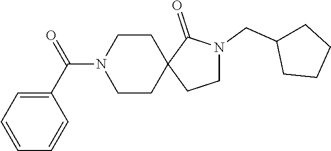 [SMILES: O=C(c1ccccc1)N1CCC2(CC1)CCN(CC1CCCC1)C2=O]

Similar to the steps described in compound 9, 8-benzoyl-2-(cyclopentylmethyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. MS(ESI)m/z: 341.2 (M+H)⁺.

### Compound 13: 8-benzoyl-2-(pyridin-3-ylmethyl)-2,8-diazaspiro[4.5]decan-1-one

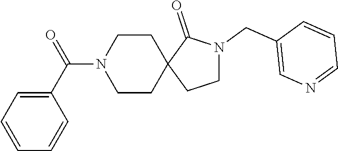 [SMILES: O=C(c1ccccc1)N1CCC2(CC1)CCN(Cc1cccnc1)C2=O]

Similar to the steps described in compound 9, 8-benzoyl-2-(pyridin-3-ylmethyl)-2,8-diazaspiro [4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (300 MHz, CDCl₃) δ 8.56 (d, J=4.9 Hz, 1H), 8.50 (s, 1H), 7.59 (d, J=7.9 Hz, 1H), 7.43-7.37 (m, 5H), 7.33-7.26 (m, 1H), 4.57-4.35 (m, 3H), 3.89-3.69 (m, 1H), 3.33-3.07 (m, 4H), 1.98 (s, 4H), 1.68-1.34 (m, 2H). MS(ESI)m/z: 350.2 (M+H)⁺.

### Compound 14: 8-benzoyl-2-(pyridin-2-ylmethyl)-2,8-diazaspiro[4.5]decan-1-one

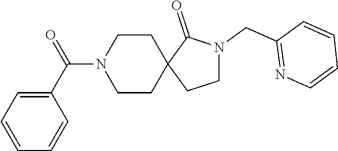 [SMILES: O=C(c1ccccc1)N1CCC2(CC1)CCN(Cc1ccccn1)C2=O]

Similar to the steps described in compound 9, 8-benzoyl-2-(pyridin-2-ylmethyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (300 MHz, CDCl₃) δ 8.53 (d, J=4.9 Hz, 1H), 7.66 (t, J=7.6 Hz, 1H), 7.41 (s, 5H), 7.20 (dd, J=7.7, 4.8 Hz, 2H), 4.60 (s, 2H), 4.52-4.29 (m, 1H), 3.92-3.74 (m, 1H), 3.42-3.14 (m, 4H), 2.16-1.80 (m, 4H), 1.62-1.36 (m, 2H). MS(ESI)m/z: 350.2 (M+H)⁺.

### Compound 15: 8-benzoyl-2-(4-(trifluoromethyl)benzyl)-2,8-diazaspiro[4.5]decan-1-one

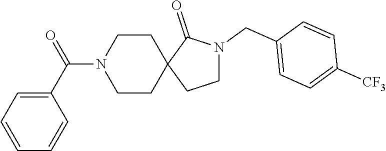 [SMILES: O=C(c1ccccc1)N1CCC2(CC1)CCN(Cc1ccc(C(F)(F)F)cc1)C2=O]

Similar to the steps described in compound 9, 8-benzoyl-2-(4-(trifluoromethyl)benzyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (300 MHz, CDCl₃) δ 7.59 (d, J=7.8 Hz, 2H), 7.46-7.38 (m, 5H), 7.33 (d, J=7.9 Hz, 2H), 4.60-4.36 (m, 3H), 3.94-3.70 (m, 1H), 3.35-3.11 (m, 4H), 2.10-1.84 (m, 4H), 1.65-1.32 (m, 2H). MS(ESI)m/z: 417.2 (M+H)⁺.

### Compound 16: 2-benzyl-8-isonicotinoyl-2,8-diazaspiro[4.5]decan-1-one

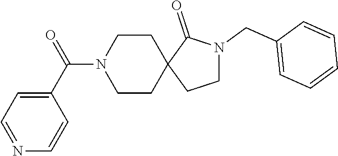 [SMILES: O=C(c1ccncc1)N1CCC2(CC1)CCN(Cc1ccccc1)C2=O]

Similar to the steps described in compound 9, 2-benzyl-8-isonicotinoyl-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (300 MHz, CDCl₃) δ 8.75-8.66 (m, 2H), 7.38-7.28 (m, 5H), 7.24-7.16 (m, 2H), 4.46 (s, 2H), 4.44-4.33 (m, 1H), 3.80-3.67 (m, 1H), 3.46-3.32 (m, 1H), 3.29-3.11 (m, 3H), 2.08-1.84 (m, 4H), 1.61-1.53 (m, 1H), 1.47-1.34 (m, 1H). MS(ESI)m/z: 350.2 (M+H)⁺.

### Compound 17: 2-benzyl-8-nicotinoyl-2,8-diazaspiro[4.5]decan-1-one

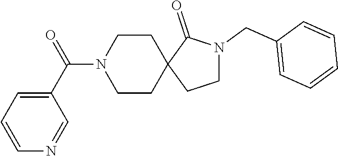 [SMILES: O=C(c1cccnc1)N1CCC2(CC1)CCN(Cc1ccccc1)C2=O]

Similar to the steps described in compound 9, 2-benzyl-8-nicotinoyl-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 8.71-8.64 (m, 2H), 7.78 (dt, J=7.9, 1.9 Hz, 1H), 7.42-7.28 (m, 4H), 7.24-7.17 (m, 2H), 4.46 (s, 2H), 4.45-4.35 (m, 1H), 3.92-3.74 (m, 1H), 3.47-3.33 (m, 1H), 3.32-3.11 (m, 3H), 2.12-1.88 (m, 4H), 1.68-1.52 (m, 1H), 1.49-1.37 (m, 1H). MS(ESI)m/z: 350.2 (M+H)⁺.

### Compound 18: 2-benzyl-8-(oxazole-5-carbonyl)-2,8-diazaspiro[4.5]decan-1-one

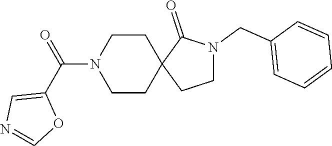 [SMILES: O=C(c1cnco1)N1CCC2(CC1)CCN(Cc1ccccc1)C2=O]

Similar to the steps described in compound 9, 2-benzyl-8-(oxazole-5-carbonyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 7.95 (s, 1H), 7.57 (s, 1H), 7.38-7.27 (m, 3H), 7.24-7.18 (m, 2H), 4.47 (s, 2H), 4.37-4.14 (m, 2H), 3.58-3.38 (m, 2H), 3.22 (t, J=6.9 Hz, 2H), 2.07-1.90 (m, 4H), 1.60-1.50 (m, 2H). MS(ESI)m/z: 340.2 (M+H)⁺.

### Compound 19: 2,8-dibenzyl-2,8-diazaspiro[4.5]decan-1-one

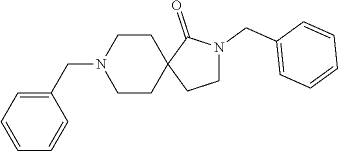 [SMILES: O=C1N(Cc2ccccc2)CCC12CCN(Cc1ccccc1)CC2]

Similar to the steps described in compound 9, 2,8-dibenzyl-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 7.80-7.63 (m, 2H), 7.50-7.39 (m, 3H), 7.38-7.24 (m, 3H), 7.18 (d, J=7.1 Hz, 2H), 4.40 (s, 2H), 4.17 (s, 2H), 3.73-3.54 (m, 2H), 3.38-3.24 (m, 2H), 3.25-3.15 (m, 2H), 2.06-1.76 (m, 6H). MS(ESI)m/z: 335.2 (M+H)⁺.

### Compound 20: 2-benzyl-8-(phenylsulfonyl)-2,8-diazaspiro [4.5]decan-1-one

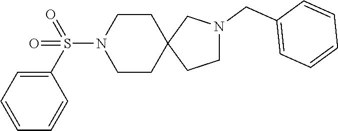 [SMILES: O=S(=O)(c1ccccc1)N1CCC2(CCN(Cc3ccccc3)C2)CC1]

Similar to the steps described in compound 9, 2-benzyl-8-(phenylsulfonyl)-2,8-diazaspiro[4.5]decan-1-one was synthesized from tert-butyl 1-oxo-2,8-diazaspiro [4.5]decane-8-carboxylate (formula 2.3) through a three-step reaction with the corresponding chemical reagents. ¹H NMR (400 MHz, CDCl₃) δ 7.85-7.65 (m, 2H), 7.63-7.39 (m, 3H), 7.36-7.21 (m, 3H), 7.21-6.98 (m, 2H), 4.47-4.25 (m, 2H), 3.65-3.40 (m, 2H), 3.18-2.98 (m, 2H), 2.96-2.69 (m, 2H), 2.08-1.85 (m, 2H), 1.82-1.72 (m, 2H), 1.63-1.40 (m, 2H). MS(ESI)m/z: 385.1 (M+H)⁺.

### Compound 21: 2-benzyl-8-methyl-2,8-diazaspiro[4.5]decan-1-one

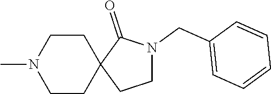 [SMILES: CN1CCC2(CC1)CCN(Cc1ccccc1)C2=O]

Similar to the steps described in compound 9, 2-benzyl-8-methyl-2,8-diazaspiro[4.5]decan-1-one was synthesized from tert-butyl 1-oxo-2,8-diazaspiro [4.5]decane-8-carboxylate (formula 2.3) through a three-step reaction with the corresponding chemical reagents. ¹H NMR (400 MHz, CDCl₃) δ 7.40-7.29 (m, 3H), 7.23-7.12 (m, 2H), 4.42 (d, J=5.1 Hz, 2H), 3.83-3.67 (m, 2H), 3.29-3.10 (m, 4H), 2.66-2.48 (m, 2H), 2.05-1.88 (m, 5H), 1.86-1.54 (m, 2H). ¹H NMR (400 MHz, CDCl₃) δ 7.40-7.29 (m, 3H), 7.24-7.17 (m, 2H), 4.42 (s, 2H), 3.80 (td, J=12.4, 3.1 Hz, 2H), 3.44-3.37 (m, 2H), 3.29-3.22 (m, 2H), 2.81 (s, 3H), 2.56-2.37 (m, 2H), 2.06-1.98 (m, 2H), 1.98-1.93 (m, 2H). MS(ESI)m/z: 259.2 (M+H)⁺.

### Compound 22: 2-benzyl-8-isopropyl-2,8-diazaspiro[4.5]decan-1-one

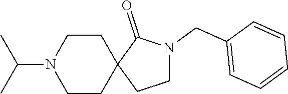 [SMILES: CC(C)N1CCC2(CCN(Cc3ccccc3)C2=O)CC1]

Similar to the steps described in compound 9, 2-benzyl-8-isopropyl-2,8-diazaspiro[4.5]decan-1-one was synthesized from tert-butyl 1-oxo-2,8-diazaspiro [4.5]decane-8-carboxylate (formula 2.3) through a three-step reaction with the corresponding chemical reagents. ¹H NMR (400 MHz, CDCl₃) δ 7.40-7.29 (m, 3H), 7.23-7.12 (m, 2H), 4.42 (d, J=5.1 Hz, 2H), 3.83-3.67 (m, 2H), 3.46 (h, J=6.6 Hz, 1H), 3.29-3.10 (m, 4H), 2.66-2.48 (m, 2H), 2.05-1.96 (m, 2H), 1.86-1.54 (m, 2H), 1.51-1.43 (m, 6H). MS(ESI)m/z: 287.2 (M+H)⁺.

### Compound 23: 2-benzyl-8-(1H-indole-3-carbonyl)-2,8-diazaspiro[4.5]decan-1-one

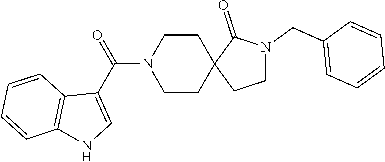 [SMILES: O=C(c1c[nH]c2ccccc12)N1CCC2(CC1)CCN(Cc1ccccc1)C2=O]

Similar to the steps described in compound 9, 2-benzyl-8-(1H-indole-3-carbonyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 9.02 (s, 1H), 7.75-7.67 (m, 1H), 7.47-7.44 (m, 1H), 7.41-7.37 (m, 1H), 7.36-7.27 (m, 3H), 7.25-7.16 (m, 4H), 4.47 (s, 2H), 4.31 (d, J=13.3 Hz, 2H), 3.30 (ddd, J=13.7, 10.6, 3.2 Hz, 2H), 3.23-3.18 (m, 2H), 2.04-1.96 (m, 4H), 1.54-1.45 (m, 2H). MS(ESI)m/z: 388.2 (M+H)⁺.

### Compound 24: 2-benzyl-8-(1H-indole-5-carbonyl)-2,8-diazaspiro[4.5]decan-1-one

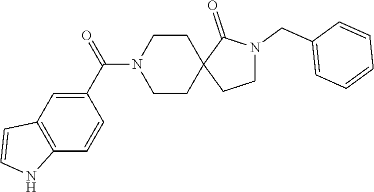 [SMILES: O=C(c1ccc2[nH]ccc2c1)N1CCC2(CC1)CCN(Cc1ccccc1)C2=O]

Similar to the steps described in compound 9, 2-benzyl-8-(1H-indole-5-carbonyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, DMSO) δ 11.31 (s, 1H), 7.66-7.56 (m, 1H), 7.47-7.40 (m, 2H), 7.38-7.31 (m, 2H), 7.30-7.24 (m, 1H), 7.22-7.17 (m, 2H), 7.16-7.11 (m, 1H), 6.51 (ddd, J=3.0, 1.9, 0.9 Hz, 1H), 4.39 (s, 2H), 4.24-3.62 (m, 2H), 3.22-3.05 (m, 4H), 1.99 (t, J=6.9 Hz, 2H), 1.76-1.62 (m, 2H), 1.49-1.36 (m, 2H). MS(ESI)m/z: 388.2 (M+H)⁺.

### Compound 25: 2-benzyl-8-(1H-indole-6-carbonyl)-2,8-diazaspiro[4.5]decan-1-one

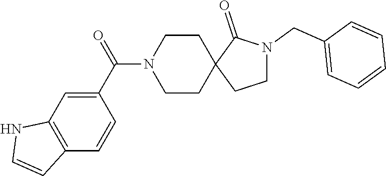 [SMILES: O=C(c1ccc2cc[nH]c2c1)N1CCC2(CC1)CCN(Cc1ccccc1)C2=O]

Similar to the steps described in compound 9, 2-benzyl-8-(1H-indole-6-carbonyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 8.97 (s, 1H), 7.65 (d, J=8.1 Hz, 1H), 7.53 (s, 1H), 7.38-7.29 (m, 4H), 7.25-7.14 (m, 3H), 6.57 (s, 1H), 4.49 (s, 2H), 4.38-3.85 (m, 2H), 3.43-3.05 (m, 4H), 2.07-1.91 (m, 4H), 1.59-1.36 (m, 2H). MS(ESI)m/z: 388.2 (M+H)⁺.

### Compound 26: 2-benzyl-8-(1-methyl-1H-benzo[d]imidazole-6-carbonyl)-2,8-diazaspiro[4.5]decan-1-one

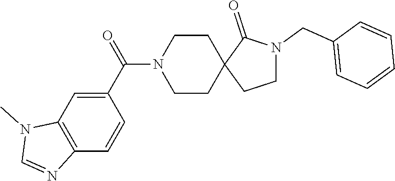 [SMILES: Cn1cnc2ccc(C(=O)N3CCC4(CC3)CCN(Cc3ccccc3)C4=O)cc21]

Similar to the steps described in compound 9, 2-benzyl-8-(1-methyl-1H-benzo[d]imidazole-6-carbonyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 7.93 (s, 1H), 7.86 (s, 1H), 7.49-7.40 (m, 2H), 7.37-7.27 (m, 3H), 7.22-7.15 (m, 2H), 4.59-4.28 (m, 3H), 4.03-3.82 (m, 4H), 3.37-3.24 (m, 2H), 3.20 (t, J=6.9 Hz, 2H), 2.07-1.87 (m, 4H), 1.59-1.36 (m, 2H). MS(ESI)m/z: 403.2 (M+H)⁺.

### Compound 27: 2-benzyl-8-(1-methyl-1H-indole-3-carbonyl)-2,8-diazaspiro[4.5]decan-1-one

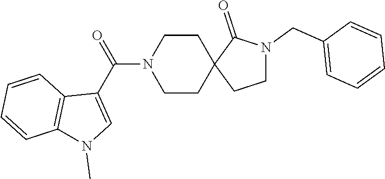 [SMILES: Cn1cc(C(=O)N2CCC3(CC2)CCN(Cc2ccccc2)C3=O)c2ccccc21]

Similar to the steps described in compound 9, 2-benzyl-8-(1-methyl-1H-indole-3-carbonyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 7.73 (dt, J=7.7, 1.1 Hz, 1H), 7.47 (s, 1H), 7.40-7.29 (m, 5H), 7.26-7.19 (m, 3H), 4.49 (s, 2H), 4.38-4.28 (m, 2H), 3.85 (s, 3H), 3.32 (ddd, J=13.7, 10.6, 3.3 Hz, 2H), 3.23 (t, J=6.9 Hz, 2H), 2.10-1.92 (m, 5H), 1.56-1.48 (m, 2H). MS(ESI)m/z: 402.2 (M+H)⁺.

### Compound 28: 2-benzyl-8-(1-methyl-1H-indole-5-carbonyl)-2,8-diazaspiro[4.5]decan-1-one

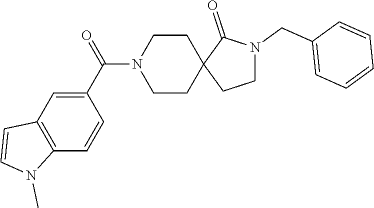 [SMILES: Cn1ccc2cc(C(=O)N3CCC4(CC3)CCN(Cc3ccccc3)C4=O)ccc21]

Similar to the steps described in compound 9, 2-benzyl-8-(1-methyl-1H-indole-5-carbonyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 7.73 (t, J=1.1 Hz, 1H), 7.35-7.32 (m, 3H), 7.32-7.27 (m, 2H), 7.23-7.18 (m, 2H), 7.11 (d, J=3.1 Hz, 1H), 6.54-6.49 (m, 1H), 4.81-4.55 (m, 2H), 4.46 (s, 2H), 3.82 (s, 3H), 3.29-3.16 (m, 4H), 2.06-1.90 (m, 4H), 1.55-1.37 (m, 2H). MS(ESI)m/z: 402.2 (M+H)⁺.

### Compound 29: 2-benzyl-8-(1-methyl-1H-indole-6-carbonyl)-2,8-diazaspiro[4.5]decan-1-one

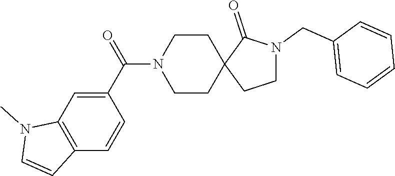 [SMILES: Cn1ccc2ccc(C(=O)N3CCC4(CC3)CCN(Cc3ccccc3)C4=O)cc21]

Similar to the steps described in compound 9, 2-benzyl-8-(1-methyl-1H-indole-6-carbonyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 7.64-7.58 (m, 1H), 7.49 (q, J=1.0 Hz, 1H), 7.36-7.27 (m, 3H), 7.23-7.18 (m, 2H), 7.16-7.11 (m, 2H), 6.50 (dd, J=3.1, 0.9 Hz, 1H), 4.46 (s, 2H), 4.34-3.91 (m, 2H), 3.83 (s, 3H), 3.30-3.13 (m, 4H), 2.08-1.90 (m, 4H), 1.78-1.63 (m, 2H). MS(ESI)m/z: 402.2 (M+H)⁺.

### Compound 30: 2-(2-fluorobenzyl)-8-(oxazole-5-carbonyl)-2,8-diazaspiro[4.5]decan-1-one

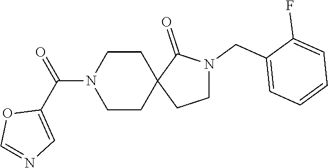 [SMILES: O=C(c1cnco1)N1CCC2(CC1)CCN(Cc1ccccc1F)C2=O]

Similar to the steps described in compound 9, 2-(2-fluorobenzyl)-8-(oxazole-5-carbonyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 7.95 (s, 1H), 7.56 (s, 1H), 7.33-7.23 (m, 3H), 7.16-7.01 (m, 2H), 4.54 (s, 2H), 4.33-4.15 (m, 2H), 3.56-3.39 (m, 2H), 3.28 (t, J=6.9 Hz, 2H), 2.07-1.94 (m, 4H), 1.75-1.62 (m, 2H), 1.60-1.50 (m, 2H). MS(ESI)m/z: 358.2 (M+H)⁺.

### Compound 31: 8-benzoyl-2-(2-fluorobenzyl)-2,8-diazaspiro[4.5]decan-1-one

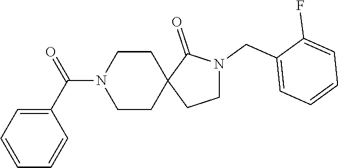 [SMILES: O=C(c1ccccc1)N1CCC2(CC1)CCN(Cc1ccccc1F)C2=O]

Similar to the steps described in compound 9, 8-benzoyl-2-(2-fluorobenzyl)-2,8-diazaspiro[4.5]decan-1-one was synthesized from tert-butyl 1-oxo-2,8-diazaspiro [4.5]decane-8-carboxylate (formula 2.3) through a three-step reaction with the corresponding chemical reagents. ¹H NMR (400 MHz, CDCl₃) δ 7.43 (s, 5H), 7.33-7.22 (m, 2H), 7.18-7.01 (m, 2H), 4.56 (s, 2H), 4.52-4.33 (m, 1H), 3.95-3.72 (m, 1H), 3.39-3.13 (m, 4H), 2.11-1.86 (m, 4H), 1.64-1.35 (m, 2H). MS(ESI)m/z: 367.2 (M+H)⁺.

### Compound 32: 8-benzoyl-2-(3-fluorobenzyl)-2,8-diazaspiro[4.5]decan-1-one

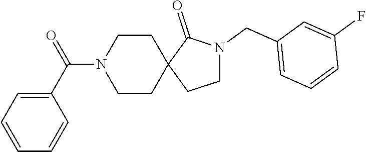 [SMILES: O=C(c1ccccc1)N1CCC2(CC1)CCN(Cc1cccc(F)c1)C2=O]

Similar to the steps described in compound 9, 8-benzoyl-2-(3-fluorobenzyl)-2,8-diazaspiro[4.5]decan-1-one was synthesized from tert-butyl 1-oxo-2,8-diazaspiro [4.5]decane-8-carboxylate (formula 2.3) through a three-step reaction with the corresponding chemical reagents. ¹H NMR (400 MHz, DMSO) δ 7.51-7.43 (m, 3H), 7.43-7.35 (m, 3H), 7.16-7.08 (m, 1H), 7.06-6.97 (m, 2H), 4.40 (s, 2H), 4.35-4.21 (m, 1H), 3.57 (s, 1H), 3.27-3.01 (m, 4H), 2.08-1.93 (m, 2H), 1.78-1.58 (m, 2H), 1.57-1.31 (m, 2H). MS(ESI)m/z: 367.2 (M+H)⁺.

### Compound 33: 8-benzoyl-2-(4-fluorobenzyl)-2,8-diazaspiro[4.5]decan-1-one

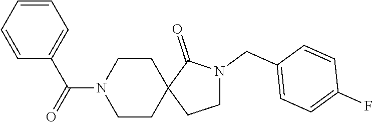 [SMILES: O=C(c1ccccc1)N1CCC2(CC1)CCN(Cc1ccc(F)cc1)C2=O]

Similar to the steps described in compound 9, 8-benzoyl-2-(4-fluorobenzyl)-2,8-diazaspiro[4.5]decan-1-one was synthesized from tert-butyl 1-oxo-2,8-diazaspiro [4.5]decane-8-carboxylate (formula 2.3) through a three-step reaction with the corresponding chemical reagents. ¹H NMR (300 MHz, DMSO) δ 7.52-7.34 (m, 5H), 7.28-7.06 (m, 4H), 4.48-4.19 (m, 3H), 3.67-3.48 (m, 1H), 3.25-2.99 (m, 4H), 2.07-1.85 (m, 2H), 1.81-1.56 (m, 2H), 1.54-1.34 (m, 2H). MS(ESI)m/z: 367.2 (M+H)⁺.

### Compound 34: 8-benzoyl-2-(2,5-difluorobenzyl)-2,8-diazaspiro[4.5]decan-1-one

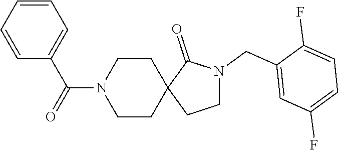 [SMILES: O=C(c1ccccc1)N1CCC2(CC1)CCN(Cc1cc(F)ccc1F)C2=O]

Similar to the steps described in compound 9, 8-benzoyl-2-(2,5-difluorobenzyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, DMSO) δ 7.50-7.42 (m, 3H), 7.42-7.35 (m, 2H), 7.32-7.24 (m, 1H), 7.24-7.16 (m, 1H), 7.09-6.99 (m, 1H), 4.43 (s, 2H), 4.33-4.21 (m, 1H), 3.66-3.49 (m, 1H), 3.29-3.21 (m, 2H), 3.19-3.01 (m, 2H), 2.08-1.91 (m, 2H), 1.78-1.58 (m, 2H), 1.55-1.31 (m, 2H). MS(ESI)m/z: 385.2 (M+H)⁺.

### Compound 35: 8-benzoyl-2-(3,5-difluorobenzyl)-2,8-diazaspiro[4.5]decan-1-one

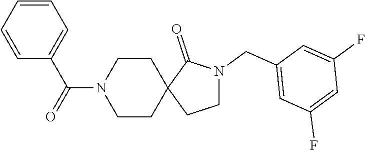 [SMILES: O=C(c1ccccc1)N1CCC2(CC1)CCN(Cc1cc(F)cc(F)c1)C2=O]

Similar to the steps described in compound 9, 8-benzoyl-2-(3,5-difluorobenzyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (300 MHz, DMSO) δ 7.42 (ddd, J=17.4, 6.5, 3.2 Hz, 5H), 7.22-7.06 (m, 1H), 6.90 (d, J=6.7 Hz, 2H), 4.40 (s, 2H), 4.36-4.16 (m, 1H), 3.74-3.46 (m, 1H), 3.29-3.19 (m, 2H), 3.19-3.01 (m, 2H), 2.09-1.90 (m, 2H), 1.75-1.57 (m, 2H), 1.57-1.32 (m, 2H). MS(ESI)m/z: 385.2 (M+H)⁺.

### Compound 36: 2-benzyl-8-(pyrimidin-2-yl)-2,8-diazaspiro[4.5]decan-1-one

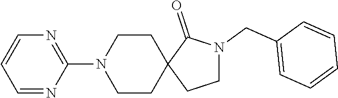 [SMILES: O=C1N(Cc2ccccc2)CCC12CCN(c1ncccn1)CC2]

Similar to the steps described in compound 9, 2-benzyl-8-(pyrimidin-2-yl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, DMSO) δ 8.36 (d, J=4.7 Hz, 2H), 7.39-7.32 (m, 2H), 7.31-7.25 (m, 1H), 7.22-7.17 (m, 2H), 6.61 (t, J=4.7 Hz, 1H), 4.52 (dt, J=13.5, 4.0 Hz, 2H), 4.39 (s, 2H), 3.21 (t, J=6.9 Hz, 2H), 3.13 (ddd, J=13.3, 11.8, 2.9 Hz, 2H), 2.02 (t, J=6.9 Hz, 2H), 1.66 (ddd, J=13.2, 11.8, 4.4 Hz, 2H), 1.47-1.38 (m, 2H). MS(ESI)m/z: 323.2 (M+H)⁺.

### Compound 37: 8-benzoyl-2-(2,6-difluorobenzyl)-2,8-diazaspiro[4.5]decan-1-one

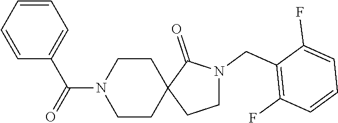 [SMILES: O=C(c1ccccc1)N1CCC2(CC1)CCN(Cc1c(F)cccc1F)C2=O]

Similar to the steps described in compound 9, 8-benzoyl-2-(2,6-difluorobenzyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 7.44-7.40 (m, 5H), 7.34-7.23 (m, 2H), 6.97-6.85 (m, 2H), 4.63 (s, 2H), 4.51-4.28 (m, 1H), 3.93-3.69 (m, 1H), 3.43-3.13 (m, 4H), 2.05-1.80 (m, 4H), 1.61-1.51 (m, 1H), 1.46-1.33 (m, 1H). MS(ESI)m/z: 385.2 (M+H)⁺.

### Compound 38: 8-benzoyl-2-(2,3,4-trifluorobenzyl)-2,8-diazaspiro[4.5]decan-1-one

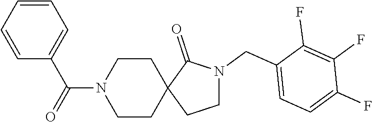 [SMILES: O=C(c1ccccc1)N1CCC2(CC1)CCN(Cc1ccc(F)c(F)c1F)C2=O]

Similar to the steps described in compound 9, 8-benzoyl-2-(2,3,4-trifluorobenzyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 7.50-7.37 (m, 5H), 7.08-6.90 (m, 2H), 4.52 (s, 2H), 4.43 (s, 1H), 3.83 (s, 1H), 3.39-3.13 (m, 4H), 2.15-1.83 (m, 4H), 1.61-1.40 (m, 2H).

### Compound 39: 8-benzoyl-2-(4-chloro-2-fluorobenzyl)-2,8-diazaspiro[4.5]decan-1-one

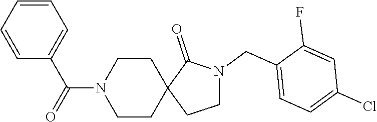 [SMILES: O=C(c1ccccc1)N1CCC2(CC1)CCN(Cc1ccc(Cl)cc1F)C2=O]

Similar to the steps described in compound 9, 8-benzoyl-2-(4-chloro-2-fluorobenzyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 7.42 (s, 5H), 7.27-7.19 (m, 1H), 7.17-7.08 (m, 2H), 4.51 (s, 2H), 4.48-4.32 (m, 1H), 3.94-3.66 (m, 1H), 3.42-3.12 (m, 4H), 2.09-1.82 (m, 4H), 1.61-1.51 (m, 1H), 1.45-1.31 (m, 1H). MS(ESI)m/z: 401.1 (M+H)⁺.

### Compound 40: 8-benzoyl-2-(2,3-difluorobenzyl)-2,8-diazaspiro[4.5]decan-1-one

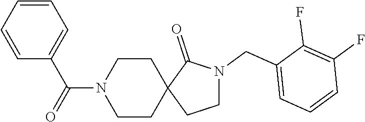 [SMILES: O=C(c1ccccc1)N1CCC2(CC1)CCN(Cc1cccc(F)c1F)C2=O]

Similar to the steps described in compound 9, 8-benzoyl-2-(2,3-difluorobenzyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 7.45-7.37 (m, 5H), 7.16-6.97 (m, 3H), 4.55 (s, 2H), 4.50-4.31 (m, 1H), 3.91-3.68 (m, 1H), 3.37-3.11 (m, 4H), 2.10-1.84 (m, 4H), 1.46-1.23 (m, 2H). MS(ESI)m/z: 385.2 (M+H)⁺.

### Compound 41: 8-benzoyl-2-(2,4,5-trifluorobenzyl)-2,8-diazaspiro[4.5]decan-1-one

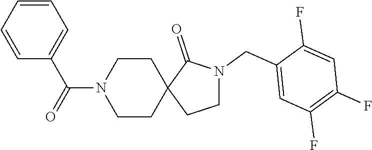 [SMILES: O=C(c1ccccc1)N1CCC2(CC1)CCN(Cc1cc(F)c(F)cc1F)C2=O]

Similar to the steps described in compound 9, 8-benzoyl-2-(2,4,5-trifluorobenzyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 7.49-7.36 (m, 5H), 7.15-7.05 (m, 1H), 6.99-6.88 (m, 1H), 4.54-4.31 (m, 3H), 3.91-3.71 (m, 1H), 3.38-3.11 (m, 4H), 2.09-1.84 (m, 4H), 1.61-1.31 (m, 2H). MS(ESI)m/z: 403.2 (M+H)⁺.

### Compound 42: 2-(2,5-difluorobenzyl)-8-(oxazole-5-carbonyl)-2,8-diazaspiro[4.5]decan-1-one

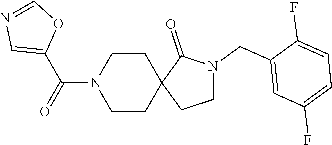 [SMILES: O=C(c1cnco1)N1CCC2(CC1)CCN(Cc1cc(F)ccc1F)C2=O]

Similar to the steps described in compound 9, 2-(2,5-difluorobenzyl)-8-(oxazole-5-carbonyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 7.95 (s, 1H), 7.57 (s, 1H), 7.08-6.90 (m, 3H), 4.51 (s, 2H), 4.33-4.17 (m, 2H), 3.59-3.40 (m, 2H), 3.30 (t, J=6.9 Hz, 2H), 2.10-1.93 (m, 4H), 1.61-1.51 (m, 2H). MS(ESI)m/z: 376.1 (M+H)⁺.

### Compound 43: 8-benzoyl-2-(2,4-difluorobenzyl)-2,8-diazaspiro[4.5]decan-1-one

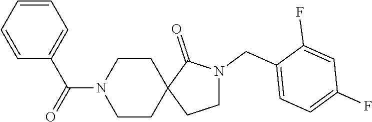 [SMILES: O=C(c1ccccc1)N1CCC2(CC1)CCN(Cc1ccc(F)cc1F)C2=O]

Similar to the steps described in compound 9, 8-benzoyl-2-(2,4-difluorobenzyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 7.95 (s, 1H), 7.57 (s, 1H), 7.08-6.90 (m, 3H), 4.51 (s, 2H), 4.33-4.17 (m, 2H), 3.59-3.40 (m, 2H), 3.30 (t, J=6.9 Hz, 2H), 2.10-1.93 (m, 4H), 1.61-1.51 (m, 2H). ¹H NMR (400 MHz, CDCl₃) δ 7.51-7.34 (m, 5H), 7.29-7.20 (m, 1H), 6.91-6.75 (m, 2H), 4.48 (s, 2H), 4.46-4.31 (m, 1H), 3.93-3.65 (m, 1H), 3.37-3.09 (m, 4H), 2.09-1.76 (m, 5H), 1.64-1.32 (m, 2H). MS(ESI)m/z: 385.2 (M+H)⁺.

### Compound 44: 2-(2-fluorobenzyl)-8-(1H-indole-6-carbonyl)-2,8-diazaspiro[4.5]decan-1-one

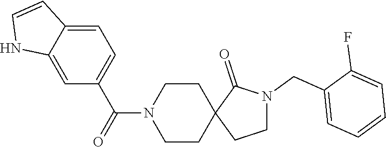 [SMILES: O=C(c1ccc2cc[nH]c2c1)N1CCC2(CC1)CCN(Cc1ccccc1F)C2=O]

Similar to the steps described in compound 9, 2-(2-fluorobenzyl)-8-(1H-indole-6-carbonyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 9.05 (s, 1H), 7.64 (d, J=8.1 Hz, 1H), 7.51 (s, 1H), 7.34-7.22 (m, 4H), 7.18-7.02 (m, 3H), 6.56 (t, J=2.7 Hz, 1H), 4.56 (s, 2H), 4.47-3.88 (m, 2H), 3.32-3.21 (m, 4H), 2.05-1.87 (m, 4H), 1.60-1.38 (m, 2H).

### Compound 45: 8-benzoyl-2-(2-chloro-6-fluorobenzyl)-2,8-diazaspiro[4.5]decan-1-one

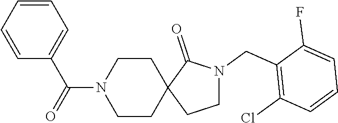 [SMILES: O=C(c1ccccc1)N1CCC2(CC1)CCN(Cc1c(F)cccc1Cl)C2=O]

Similar to the steps described in compound 9, 8-benzoyl-2-(2-chloro-6-fluorobenzyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 7.49-7.34 (m, 5H), 7.28-7.17 (m, 2H), 7.08-6.93 (m, 1H), 4.70 (s, 2H), 4.51-4.24 (m, 1H), 3.98-3.67 (m, 1H), 3.37-3.06 (m, 4H), 2.07-1.79 (m, 4H), 1.65-1.33 (m, 2H). MS(ESI)m/z: 401.1 (M+H)⁺.

### Compound 46: 2-(2,5-difluorobenzyl)-8-(1H-indole-6-carbonyl)-2,8-diazaspiro[4.5]decan-1-one

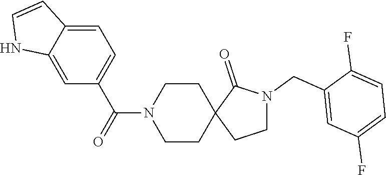 [SMILES: O=C(c1ccc2cc[nH]c2c1)N1CCC2(CC1)CCN(Cc1cc(F)ccc1F)C2=O]

Similar to the steps described in compound 9, 2-(2,5-difluorobenzyl)-8-(1H-indole-6-carbonyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 8.59 (s, 1H), 7.66 (d, J=8.1 Hz, 1H), 7.55 (s, 1H), 7.35-7.30 (m, 1H), 7.18 (dd, J=8.1, 1.4 Hz, 1H), 7.08-6.90 (m, 3H), 6.64-6.55 (m, 1H), 4.53 (s, 2H), 4.47-3.86 (m, 2H), 3.34-3.22 (m, 4H), 2.09-1.94 (m, 4H), 1.57-1.43 (m, 2H). MS(ESI)m/z: 424.2 (M+H)⁺.

### Compound 47: 8-benzoyl-2-(5-chloro-2-fluorobenzyl)-2,8-diazaspiro[4.5]decan-1-one

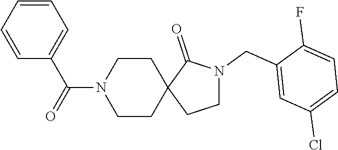 [SMILES: O=C(c1ccccc1)N1CCC2(CC1)CCN(Cc1cc(Cl)ccc1F)C2=O]

Similar to the steps described in compound 9, 8-benzoyl-2-(5-chloro-2-fluorobenzyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 7.47-7.35 (m, 5H), 7.26-7.17 (m, 2H), 7.00 (t, J=8.9 Hz, 1H), 4.58-4.27 (m, 3H), 3.99-3.72 (m, 1H), 3.37-3.08 (m, 4H), 2.14-1.83 (m, 4H), 1.63-1.34 (m, 2H). MS(ESI)m/z: 401.1 (M+H)⁺.

### Compound 48: 4-((8-benzoyl-1-oxo-2,8-diazaspiro[4.5]decan-2-yl)methyl)-3-fluorobenzonitrile

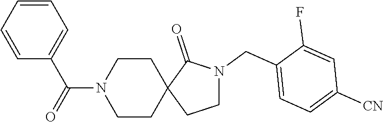 [SMILES: N#Cc1ccc(CN2CCC3(CCN(C(=O)c4ccccc4)CC3)C2=O)c(F)c1]

Similar to the steps described in compound 9, 4-((8-benzoyl-1-oxo-2,8-diazaspiro[4.5]decan-2-yl)methyl)-3-fluorobenzonitrile was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 7.46-7.34 (m, 8H), 4.57 (s, 2H), 4.50-4.32 (m, 1H), 3.90-3.72 (m, 1H), 3.38-3.08 (m, 4H), 2.13-1.79 (m, 4H), 1.65-1.37 (m, 2H). MS(ESI)m/z: 392.2 (M+H)⁺.

### Compound 49: 2-(2,5-difluorobenzyl)-8-(1-methyl-1H-indole-6-carbonyl)-2,8-diazaspiro[4.5]decan-1-one

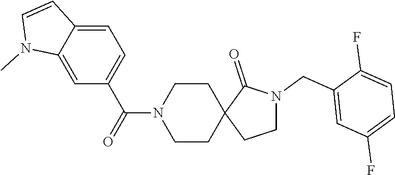 [SMILES: Cn1ccc2ccc(C(=O)N3CCC4(CC3)CCN(Cc3cc(F)ccc3F)C4=O)cc21]

Similar to the steps described in compound 9, 2-(2,5-difluorobenzyl)-8-(1-methyl-1H-indole-6-carbonyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 7.60 (d, J=8.1 Hz, 1H), 7.48 (s, 1H), 7.20-7.08 (m, 2H), 7.06-6.87 (m, 3H), 6.49 (s, 1H), 4.50 (s, 2H), 4.39-3.96 (m, 2H), 3.81 (s, 3H), 3.44-3.16 (m, 4H), 2.11-1.87 (m, 4H), 1.59-1.36 (m, 2H). MS(ESI)m/z: 438.2 (M+H)⁺.

### Compound 50: 2-(2-fluorobenzyl)-8-(1-methyl-1H-indole-6-carbonyl)-2,8-diazaspiro decan-1-one

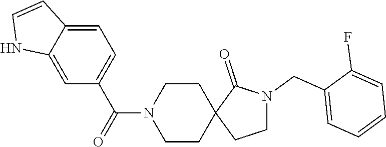 [SMILES: O=C(c1ccc2cc[nH]c2c1)N1CCC2(CC1)CCN(Cc1ccccc1F)C2=O]

Similar to the steps described in compound 9, 2-(2-fluorobenzyl)-8-(1-methyl-1H-indole-6-carbonyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 7.62 (d, J=8.1 Hz, 1H), 7.50 (s, 1H), 7.34-7.22 (m, 2H), 7.19-7.02 (m, 4H), 6.51 (d, J=3.1 Hz, 1H), 4.56 (s, 2H), 4.41-3.99 (m, 2H), 3.83 (s, 3H), 3.34-3.20 (m, 4H), 2.08-1.92 (m, 4H), 1.59-1.38 (m, 2H). MS(ESI)m/z: 420.2 (M+H)⁺.

### Compound 51: 2-(2,5-difluorobenzyl)-8-(pyrimidin-2-yl)-2,8-diazaspiro[4.5]decan-1-one

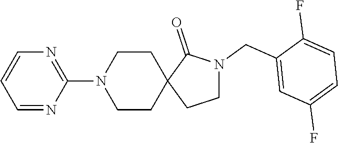 [SMILES: O=C1N(Cc2cc(F)ccc2F)CCC12CCN(c1ncccn1)CC2]

Similar to the steps described in compound 9, 2-(2,5-difluorobenzyl)-8-(pyrimidin-2-yl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 8.30 (d, J=4.7 Hz, 2H), 7.12-6.84 (m, 3H), 6.47 (t, J=4.8 Hz, 1H), 4.60 (dt, J=13.8, 4.4 Hz, 2H), 4.51 (s, 2H), 3.33-3.19 (m, 4H), 2.06 (t, J=6.9 Hz, 2H), 1.97 (ddd, J=13.5, 11.2, 4.3 Hz, 2H), 1.49 (dt, J=13.1, 3.4 Hz, 2H). MS(ESI)m/z: 359.2 (M+H)⁺.

### Compound 52: 2-(2-(2,5-difluorobenzyl)-1-oxo-2,8-diazaspiro[4.5]decan-8-yl) pyrimidine-4-carbonitrile

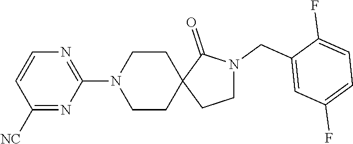 [SMILES: N#Cc1ccnc(N2CCC3(CCN(Cc4cc(F)ccc4F)C3=O)CC2)n1]

Similar to the steps described in compound 9, 2-(2-(2,5-difluorobenzyl)-1-oxo-2,8-diazaspiro[4.5]decan-8-yl)pyrimidine-4-carbonitrile was synthesized from tert-butyl 1-oxo-2,8-diazaspiro [4.5]decane-8-carboxylate (formula 2.3) through a three steps reaction with the corresponding chemical reagents. ¹H NMR (300 MHz, CDCl₃) δ 8.43 (d, J=4.6 Hz, 1H), 7.10-6.89 (m, 3H), 6.73 (d, J=4.6 Hz, 1H), 4.60-4.46 (m, 4H), 3.42-3.25 (m, 4H), 2.05 (t, J=6.9 Hz, 2H), 2.00-1.88 (m, 2H), 1.57-1.43 (m, 3H). MS(ESI)m/z: 384.2 (M+H)⁺.

### Compound 53: 8-benzoyl-2-(2,3,5-trifluorobenzyl)-2,8-diazaspiro[4.5]decan-1-one

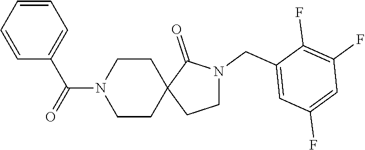 [SMILES: O=C(c1ccccc1)N1CCC2(CC1)CCN(Cc1cc(F)cc(F)c1F)C2=O]

Similar to the steps described in compound 9, 8-benzoyl-2-(2,3,5-trifluorobenzyl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (300 MHz, CDCl₃) δ 7.52-7.34 (m, 5H), 6.93-6.80 (m, 1H), 6.81-6.68 (m, 1H), 4.62-4.31 (m, 3H), 3.92-3.69 (m, 1H), 3.39-3.08 (m, 4H), 2.14-1.83 (m, 4H), 1.57-1.33 (m, 2H). MS(ESI)m/z: 401.1 (M+H)⁺. MS(ESI)m/z: 403.1 (M+H)⁺.

### Compound 54: 2-((8-benzoyl-1-oxo-2,8-diazaspiro[4.5]decan-2-yl)methyl) benzonitrile

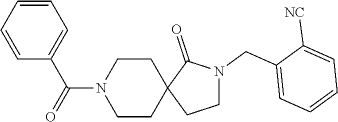 [SMILES: N#Cc1ccccc1CN1CCC2(CCN(C(=O)c3ccccc3)CC2)C1=O]

Similar to the steps described in compound 9, 2-((8-benzoyl-1-oxo-2,8-diazaspiro[4.5]decan-2-yl)methyl)benzonitrile was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 7.67 (d, J=7.7 Hz, 1H), 7.59 (t, J=7.8 Hz, 1H), 7.49-7.33 (m, 7H), 4.70 (s, 2H), 4.53-4.32 (m, 1H), 3.94-3.71 (m, 1H), 3.39-3.12 (m, 4H), 2.12-1.88 (m, 4H), 1.61-1.37 (m, 2H). MS(ESI)m/z: 374.2 (M+H)⁺.

### Compound 55: 2-(2,5-difluorobenzyl)-8-(5-methyl-1,3,4-oxadiazol-2-yl)-2,8-diazaspiro[4.5]decan-1-one

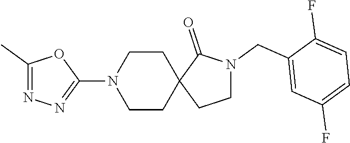 [SMILES: Cc1nnc(N2CCC3(CCN(Cc4cc(F)ccc4F)C3=O)CC2)o1]

Similar to the steps described in compound 9, 2-(2,5-difluorobenzyl)-8-(5-methyl-1,3,4-oxadiazol-2-yl)-2,8-diazaspiro[4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (300 MHz, CDCl₃) δ 7.10-6.82 (m, 3H), 4.50 (s, 2H), 4.01-3.86 (m, 2H), 3.34-3.17 (m, 4H), 2.39 (s, 3H), 2.10-1.93 (m, 4H), 1.60-1.46 (m, 2H). MS(ESI)m/z: 363.2 (M+H)⁺.

### Compound 56: 6-(2-(2,5-difluorobenzyl)-1-oxo-2,8-diazaspiro[4.5]decan-8-yl)pyridazine-3-carbonitrile

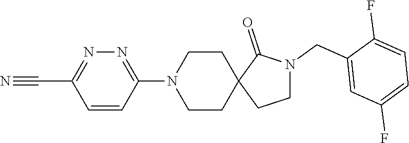 [SMILES: N#Cc1ccc(N2CCC3(CCN(Cc4cc(F)ccc4F)C3=O)CC2)nn1]

Similar to the steps described in compound 9, 6-(2-(2,5-difluorobenzyl)-1-oxo-2,8-diazaspiro[4.5]decan-8-yl)pyridazine-3-carbonitrile was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 8.00 (d, J=9.6 Hz, 1H), 7.09-6.89 (m, 4H), 4.52 (s, 2H), 4.41-4.31 (m, 2H), 3.53-3.45 (m, 3H), 3.31 (t, J=6.9 Hz, 2H), 2.11-1.99 (m, 4H), 1.62-1.54 (m, 2H).

### Compound 57: 2-((8-benzoyl-1-oxo-2,8-diazaspiro[4.5]decan-2-yl)methyl)-4-fluorobenzonitrile

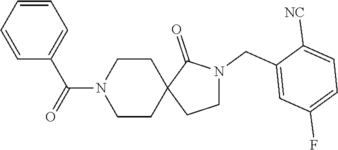 [SMILES: N#Cc1ccc(F)cc1CN1CCC2(CCN(C(=O)c3ccccc3)CC2)C1=O]

Similar to the steps described in compound 9, 2-((8-benzoyl-1-oxo-2,8-diazaspiro[4.5]decan-2-yl)methyl)-4-fluorobenzonitrile was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 7.69 (s, 1H), 7.60-7.25 (m, 5H), 7.19-6.89 (m, 2H), 4.68 (s, 2H), 4.57-4.29 (m, 1H), 4.00-3.63 (m, 1H), 3.53-3.04 (m, 4H), 2.28-1.73 (m, 4H), 1.71-1.36 (m, 2H). MS(ESI)m/z: 392.2 (M+H)⁺.

### Compound 58: 6-(2-(2,5-difluorobenzyl)-1-oxo-2,8-diazaspiro[4.5]decan-8-yl)pyrimidine-4-carbonitrile

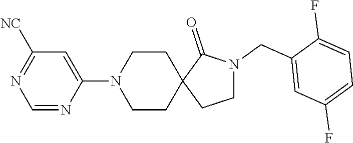 [SMILES: N#Cc1cc(N2CCC3(CCN(Cc4cc(F)ccc4F)C3=O)CC2)ncn1]

Similar to the steps described in compound 9, 6-(2-(2,5-difluorobenzyl)-1-oxo-2,8-diazaspiro[4.5]decan-8-yl)pyrimidine-4-carbonitrile was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 8.58 (d, J=1.1 Hz, 1H), 7.11-6.92 (m, 3H), 6.88 (d, J=1.3 Hz, 1H), 4.51 (s, 2H), 4.40-4.02 (m, 2H), 3.57-3.39 (m, 2H), 3.32 (t, J=6.9 Hz, 2H), 2.07-1.92 (m, 4H), 1.62-1.52 (m, 2H). MS(ESI)m/z: 401.1 (M+H)⁺. MS(ESI)m/z: 384.1 (M+H)⁺.

### Example 3

Compounds with a structure of formula 3.5 can be prepared through reaction route 3.

### Reaction Route 3

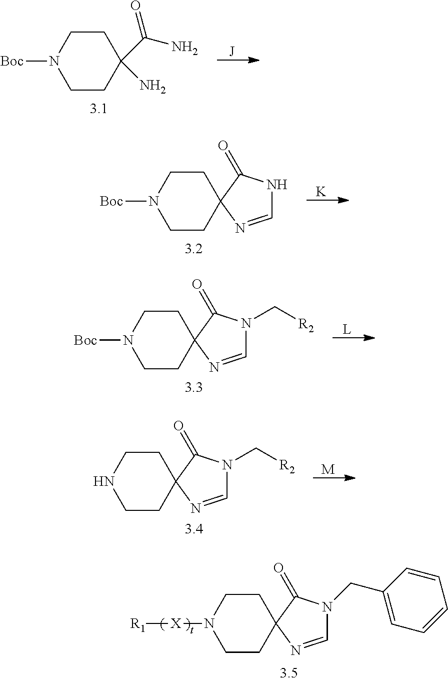

The preparation method of the key intermediate tert-butyl 4-oxo-1,3,8-triazaspiro[4.5]dec-1-ene-8-carboxylate (formula 3.2) is as follows:

Step J: The commercially 4-amino-4-carbamylpiperidin-1-tert-butyl carboxylate (500 mg, 2.06 mmol) was suspended in toluene, and trimethyl orthoformate (654 mg, 6.17 mmol) and acetic acid (617 mg, 10.28 mmol) were added to the mixture and stirred at 90° C. for 12 h. When TLC showed that the reaction was completed, the reaction liquid was cooled to room temperature. The solvent was removed under reduced pressure, and the residue was extracted with ethyl acetate. The organic phase was washed with brine, and dried over anhydrous Na₂SO₄. Purification of the crude material by silica gel chromatography to obtain the intermediate 4-oxo-1,3,8-triazospiro [4.5] deca-1-en-8-carboxylic acid tert butyl ester (450 mg, 86%). ¹H NMR (300 MHz, CDCl₃) δ 8.68 (s, 1H), 7.78 (s, 1H), 4.17-3.90 (m, 2H), 3.45-3.24 (m, 2H), 1.94-1.79 (m, 2H), 1.52-1.47 (m, 10H), 1.45-1.39 (m, 1H).

### Compound 59: 8-benzoyl-3-benzyl-1,3,8-triazaspiro[4.5]dec-1-en-4-one

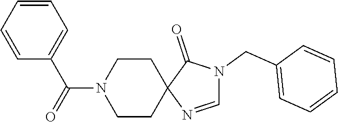 [SMILES: O=C(c1ccccc1)N1CCC2(CC1)N=CN(Cc1ccccc1)C2=O]

Step K: 4-Oxo-1,3,8-triazospiro [4.5] deca-1-en-8-carboxylic acid tert butyl ester (65 mg, 0.26 mmol) was dissolved in DMF, sodium hydroxide (30 mg, 0.77 mmol) and benzyl bromide (39 mg, 0.31 mmol) were added, and stirred at room temperature for 4 h. TLC showed that the reaction was completed, and the reaction mixture was partitioned between water and EtOAc. The organic layers were washed with water, and brine, dried over anhydrous Na₂SO₄, and then purification of the crude material by silica gel chromatography to obtain 3-benzyl-4-oxo-1,3,8-triazospiro [4.5] deca-1-en-8-carboxylic acid tert butyl ester (73 mg, 83%).

Step L: 65 mg of 3-benzyl-4-oxo-1,3,8-triazospiro [4.5] deca-1-en-8-carboxylic acid tert butyl ester was dissolved in 1 mL dichloromethane, and 0.3 mL of trifluoroacetic acid was added for 2 h at room temperature. After the reaction was completed, dilute with dichloromethane and wash with saturated NaHCO₃. The DCM layer was dried with anhydrous Na₂SO₄ and concentrated until dry to obtain 3-benzyl-1,3,8-triazaspirocyclic [4.5]deca-1-en-4-one.

Step M: 3-benzyl-1,3,8-triazaspirocyclic [4.5] deca-1-en-4-one (207 mg, 0.85 mmol) was dissolved in dichloromethane (5 mL), added triethylamine (422 μL, 2.55 mmol) and then added benzoyl chloride (118.60 μL, 1.02 mmol) under an ice bath, stirred for 1 hour. After the reaction was completed, the reaction mixture was washed with saturated ammonium chloride solution, washed with water, brine, and dried over anhydrous Na₂SO₄, and then purification of the crude material by silica gel chromatography to obtain 8-benzoyl-3-benzyl-1,3,8-triazaspiro[4.5]dec-1-en-4-one. ¹H NMR (300 MHz, CDCl₃) δ 7.65 (s, 1H), 7.48-7.29 (m, 8H), 7.25-7.17 (m, 2H), 4.65 (s, 2H), 4.64-4.51 (m, 1H), 3.87-3.69 (m, 1H), 3.61-3.43 (m, 2H), 2.12-1.82 (m, 2H), 1.66-1.36 (m, 2H). MS(ESI)m/z: 348.2 (M+H)⁺.

### Compound 60: 8-benzoyl-3-(2-fluorobenzyl)-1,3,8-triazaspiro[4.5]dec-1-en-4-one

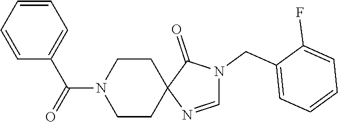 [SMILES: O=C(c1ccccc1)N1CCC2(CC1)N=CN(Cc1ccccc1F)C2=O]

Similar to the steps described in compound 59, 8-benzoyl-3-(2-fluorobenzyl)-1,3,8-triazaspiro[4.5]dec-1-en-4-one was synthesized from tert-butyl 4-oxo-1,3,8-triazaspiro[4.5]dec-1-ene-8-carboxylate (formula 3.2) through a three steps reaction with the corresponding chemical reagents. ¹H NMR (300 MHz, CDCl₃) δ 7.73 (s, 1H), 7.42 (s, 5H), 7.37-7.24 (m, 2H), 7.12 (dt, J=17.6, 8.3 Hz, 2H), 4.69 (s, 2H), 4.65-4.48 (m, 1H), 3.87-3.67 (m, 1H), 3.62-3.37 (m, 2H), 1.95-1.78 (m, 2H), 1.67-1.51 (m, 1H), 1.45-1.34 (m, 1H). MS(ESI)m/z: 366.1 (M+H)⁺.

### Compound 61: 8-benzoyl-3-(2,5-difluorobenzyl)-1,3,8-triazaspiro[4.5]dec-1-en-4-one

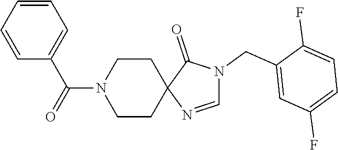 [SMILES: O=C(c1ccccc1)N1CCC2(CC1)N=CN(Cc1cc(F)ccc1F)C2=O]

Similar to the steps described in compound 59, 8-benzoyl-3-(2,5-difluorobenzyl)-1,3,8-triazaspiro[4.5]dec-1-en-4-one as synthesized from tert-butyl 4-oxo-1,3,8-triazaspiro[4.5]dec-1-ene-8-carboxylate (formula 3.2) through a three steps reaction with the corresponding chemical reagents. ¹H NMR (400 MHz, CDCl₃) δ 7.78 (s, 1H), 7.50-7.34 (m, 5H), 7.14-6.94 (m, 3H), 4.68 (s, 2H), 4.64-4.53 (m, 1H), 3.87-3.72 (m, 1H), 3.63-3.43 (m, 2H), 2.12-1.98 (m, 1H), 1.96-1.81 (m, 1H), 1.70-1.52 (m, 1H), 1.49-1.35 (m, 1H). MS(ESI)m/z: 384.1 (M+H)⁺.

### Example 4

Compounds with a structure of formula 4.6 can be prepared through reaction route 4.

### Reaction Route 4

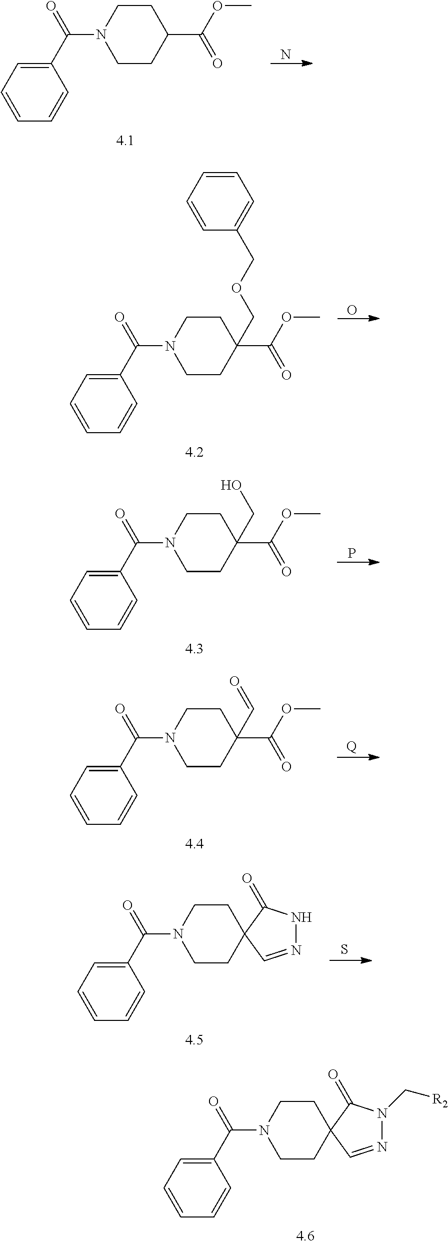

The preparation method of the key intermediate 8-benzoyl-2,3,8-triazaspiro[4.5]dec-3-en-1-one (formula 4.5) is as follows:

Step N: 1-Benzoylpiperidin-4-carboxylate methyl ester (2.4 g, 9.71 mmol) was dissolved in dry tetrahydrofuran, and a solution of LDA in tetrahydrofuran (2 M, 7.28 mL, 14.56 mmol) was added at −78° C. and stirred at −78° C. for 1 h, then chloromethyl benzyl ether (2.7 mL, 19.41 mmol) was added and stirred at −78° C. for 4 h. Then slowly rose to room temperature and continued for 6 h. After the reaction was completed, the reaction mixture was concentrated and added with ethyl acetate, the ethyl acetate layer was washed with saturated ammonium chloride solution, brine dried over anhydrous Na₂SO₄, and then purified by column chromatography to obtain 1-benzoyl-4-((benzyloxy) methyl) piperidine-4-carboxylate methyl ester (2.15 g, 60%). ¹H NMR (400 MHz, CDCl₃) δ 7.44-7.28 (m, 8H), 7.28-7.27 (m, 1H), 7.26-7.24 (m, 1H), 4.49 (s, 2H), 4.41 (d, J=13.7 Hz, 1H), 3.74 (s, 3H), 3.58 (t, J=10.8 Hz, 1H), 3.54-3.38 (m, 2H), 3.22 (s, 1H), 3.04 (s, 1H), 2.27-2.09 (m, 2H), 1.65-1.54 (m, 1H), 1.51-1.38 (m, 1H).

Step O: 1-Benzoyl-4-((benzoxy) methyl) piperidine-4-carboxylate methyl ester (2.52 g, 6.86 mmol) was dissolved in methanol, palladium carbon (10%, 200 mg) was added, hydrogen was substituted for 3 times, and then stirred at 35° C. for 12 h. After the reaction was completed, filtered with diatomaceous earth. The filtrate was concentrated and dried to obtain 1-benzoyl-4-(hydroxymethyl) piperidine-4-carboxylate methyl ester, which was directly used in the next reaction. ¹H NMR (400 MHz, CDCl₃) δ 7.45-7.35 (m, 5H), 4.41-4.19 (m, 1H), 3.78 (s, 3H), 3.74-3.52 (m, 3H), 3.33-3.15 (m, 2H), 2.26-2.13 (m, 1H), 2.13-1.96 (m, 2H), 1.67-1.54 (m, 1H), 1.52-1.38 (m, 1H).

Step P: Oxaloyl chloride (615 μL, 7.21 mmol) was dissolved in dichloromethane, and DMSO (1.02 ml, 14.42 mmol) was added to dichloromethane solution under a nitrogen atmosphere, and stirred at −78° C. for 30 min. 1-benzoyl-4-(hydroxymethyl) piperidine-4-carboxylate methyl ester (1 g, 3.61 mmol) was dissolved in dichloromethane (4 mL) and added to the reaction system at −78° C. After 2 hours, the DIPEA (3 mL, 18.03 mmol) was added for 1 h. After the reaction was completed, TLC analysis indicated that the reaction was complete, the mixture was washed with water, brine, and dried over anhydrous Na₂SO₄. Then purified by column chromatography to obtain 1-benzoyl-4-formylpiperidin-4-carboxylate methyl ester (970 mg, 98%). ¹H NMR (400 MHz, DMSO) δ 9.60 (s, 1H), 7.47-7.34 (m, 5H), 3.80 (s, 5H), 3.56-3.33 (m, 2H), 2.15-1.87 (m, 4H).

Step Q: 1-Benzoyl-4-formylpiperidin-4-carboxylate methyl ester (500 mg) in methanol (5 mL) and hydrazine hydrate (98%, 300 μL) was added and stirred overnight at 40° C. After t the reaction was completed, the reaction mixture was concentrated and dissolved with DCM. The organic phase was washed with water, and brine, and dried over anhydrous Na₂SO₄, and purified by column chromatography on silica gel to provide the 8-benzoyl-2,3,8-triazaspiro[4.5]dec-3-en-1-one (formula 4.5). ¹H NMR (400 MHz, DMSO) δ 11.35 (s, 1H), 7.81-7.73 (m, 1H), 7.50-7.31 (m, 5H), 4.34-4.08 (m, 1H), 3.77-3.57 (m, 1H), 3.52-3.36 (m, 2H), 1.73-1.45 (m, 4H).

### Compound 62: 8-benzoyl-2-benzyl-2,3,8-triazaspiro[4.5]dec-3-en-1-one

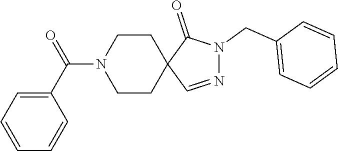 [SMILES: O=C(c1ccccc1)N1CCC2(C=NN(Cc3ccccc3)C2=O)CC1]

8-Benzoyl-2,3,8-triazine [4.5]decan-3-ene-1-one (290 mg, 1.13 mmol), potassium hydroxide (76 mg, 1.35 mmol) and benzyl bromide (161 μL, 1.35 mmol) was dissolved in DMF (5 ml). The mixture was stirred at room temperature for 4 h. The reaction mixture was partitioned using ethyl acetate and water. The organic layer was washed with water, brine, dried over anhydrous Na₂SO₄, and purified by column chromatography on silica gel to provide the 8-benzoyl-2-benzyl-2,3,8-triazaspiro [4.5]decan-3-ene-1-one (340 mg, 87%). ¹H NMR (400 MHz, CDCl₃) δ 7.94 (s, 1H), 7.56-7.39 (m, 5H), 7.38-7.13 (m, 5H), 4.81 (s, 2H), 4.35-4.13 (m, 1H), 3.77-3.58 (m, 1H), 3.55-3.39 (m, 2H), 1.83-1.54 (m, 4H). MS(ESI)m/z: 348.1 (M+H)⁺.

### Compound 63: 8-benzoyl-2-(2-fluorobenzyl)-2,3,8-triazaspiro[4.5]dec-3-en-1-one

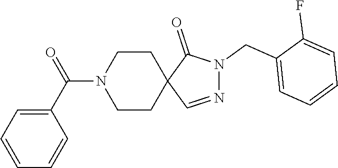 [SMILES: O=C(c1ccccc1)N1CCC2(C=NN(Cc3ccccc3F)C2=O)CC1]

Similar to the steps described in compound 62, 8-benzoyl-2-(2-fluorobenzyl)-2,3,8-triazaspiro[4.5]dec-3-en-1-one was synthesized from 8-benzoyl-2,3,8-triazaspiro[4.5]dec-3-en-1-one (formula 4.5) through the reaction with the corresponding chemical reagents. ¹H NMR (400 MHz, CDCl₃) δ 7.50-7.39 (m, 5H), 7.38 (s, 1H), 7.34-7.27 (m, 1H), 7.10-7.03 (m, 1H), 7.02-6.93 (m, 2H), 4.84 (s, 2H), 4.44-4.19 (m, 1H), 4.08-3.83 (m, 1H), 3.79-3.21 (m, 2H), 1.96-1.58 (m, 4H). MS(ESI)m/z: 366.2 (M+H)⁺.

### Compound 64: 8-benzoyl-2-(3-fluorobenzyl)-2,3,8-triazaspiro[4.5]dec-3-en-1-one

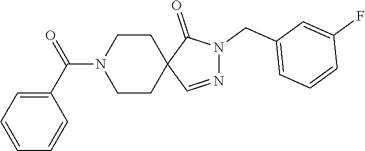 [SMILES: O=C(c1ccccc1)N1CCC2(C=NN(Cc3cccc(F)c3)C2=O)CC1]

Similar to the steps described in compound 62, 8-benzoyl-2-(3-fluorobenzyl)-2,3,8-triazaspiro [4.5]dec-3-en-1-one was synthesized from 8-benzoyl-2,3,8-triazaspiro[4.5]dec-3-en-1-one (formula 4.5) through the reaction with the corresponding chemical reagents. ¹H NMR (400 MHz, CDCl₃) δ 7.45 (d, J=2.9 Hz, 5H), 7.40 (s, 1H), 7.36-7.30 (m, 1H), 7.16-7.06 (m, 1H), 7.06-6.95 (m, 2H), 4.87 (s, 2H), 4.55-4.21 (m, 1H), 4.10-3.87 (m, 1H), 3.84-3.28 (m, 2H), 2.02-1.63 (m, 4H). MS(ESI)m/z: 366.2 (M+H)⁺.

### Compound 65: 8-benzoyl-2-(4-fluorobenzyl)-2,3,8-triazaspiro[4.5]dec-3-en-1-one

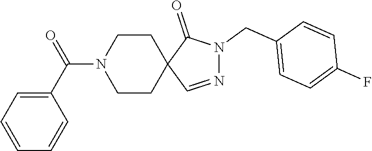 [SMILES: O=C(c1ccccc1)N1CCC2(C=NN(Cc3ccc(F)cc3)C2=O)CC1]

Similar to the steps described in compound 62, 8-benzoyl-2-(4-fluorobenzyl)-2,3,8-triazaspiro[4.5]dec-3-en-1-one was synthesized from 8-benzoyl-2,3,8-triazaspiro[4.5]dec-3-en-1-one (formula 4.5) through the reaction with the corresponding chemical reagents. ¹H NMR (400 MHz, CDCl₃) δ 7.46-7.39 (m, 5H), 7.36 (s, 1H), 7.31-7.27 (m, 1H), 7.07-6.97 (m, 2H), 4.81 (s, 2H), 4.49-4.16 (m, 1H), 4.06-3.83 (m, 1H), 3.81-3.24 (m, 2H), 1.98-1.63 (m, 4H). MS(ESI)m/z: 366.2 (M+H)⁺.

### Compound 66: 8-benzoyl-2-(2,5-difluorobenzyl)-2,3,8-triazaspiro[4.5]dec-3-en-1-one

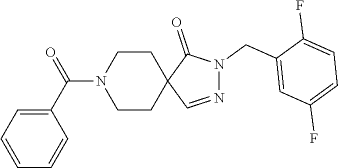 [SMILES: O=C(c1ccccc1)N1CCC2(C=NN(Cc3cc(F)ccc3F)C2=O)CC1]

Similar to the steps described in compound 62, 8-benzoyl-2-(2,5-difluorobenzyl)-2,3,8-triazaspiro[4.5]dec-3-en-1-one was synthesized from 8-benzoyl-2,3,8-triazaspiro[4.5]dec-3-en-1-one (formula 4.5) through the reaction with the corresponding chemical reagents. ¹H NMR (400 MHz, CDCl₃) δ 7.45-7.40 (m, 5H), 7.39 (s, 1H), 7.08-6.88 (m, 3H), 4.90 (s, 2H), 4.49-4.19 (m, 1H), 4.09-3.85 (m, 1H), 3.82-3.29 (m, 2H), 1.99-1.66 (m, 4H). MS(ESI)m/z: 384.2 (M+H)⁺.

### Compound 67: 8-benzoyl-2-(3,5-difluorobenzyl)-2,3,8-triazaspiro[4.5]dec-3-en-1-one

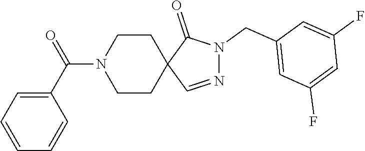 [SMILES: O=C(c1ccccc1)N1CCC2(C=NN(Cc3cc(F)cc(F)c3)C2=O)CC1]

Similar to the steps described in compound 62, 8-benzoyl-2-(3,5-difluorobenzyl)-2,3,8-triazaspiro[4.5]dec-3-en-1-one was synthesized from 8-benzoyl-2,3,8-triazaspiro[4.5]dec-3-en-1-one (formula 4.5) through the reaction with the corresponding chemical reagents. ¹H NMR (400 MHz, CDCl₃) δ 7.48-7.42 (m, 5H), 7.42 (s, 1H), 6.88-6.80 (m, 2H), 6.79-6.72 (m, 1H), 4.84 (s, 2H), 4.50-4.20 (m, 1H), 4.08-3.84 (m, 1H), 3.84-3.30 (m, 2H), 2.05-1.64 (m, 4H). MS(ESI)m/z: 384.2 (M+H)⁺.

### Compound 68: 8-benzoyl-2-(3-(trifluoromethyl)benzyl)-2,3,8-triazaspiro[4.5]dec-3-en-1-one

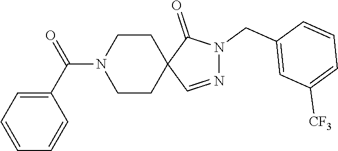 [SMILES: O=C(c1ccccc1)N1CCC2(C=NN(Cc3cccc(C(F)(F)F)c3)C2=O)CC1]

Similar to the steps described in compound 62, 8-benzoyl-2-(3-(trifluoromethyl)benzyl)-2,3,8-triazaspiro[4.5]dec-3-en-1-one was synthesized from 8-benzoyl-2,3,8-triazaspiro[4.5]dec-3-en-1-one (formula 4.5) through the reaction with the corresponding chemical reagents. ¹H NMR (400 MHz, DMSO) δ 7.96 (s, 1H), 7.76-7.20 (m, 10H), 4.92 (s, 2H), 4.34-4.00 (m, 1H), 3.84-3.56 (m, 1H), 3.55-3.39 (m, 2H), 1.83-1.46 (m, 4H). MS(ESI)m/z: 416.2 (M+H)⁺.

### Compound 69: 4-((8-benzoyl-1-oxo-2,3,8-triazaspiro[4.5]dec-3-en-2-yl)methyl) benzonitrile

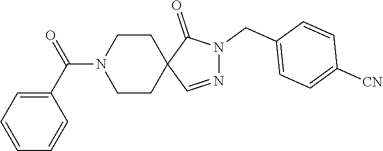 [SMILES: N#Cc1ccc(CN2N=CC3(CCN(C(=O)c4ccccc4)CC3)C2=O)cc1]

Similar to the steps described in compound 62, 4-((8-benzoyl-1-oxo-2,3,8-triazaspiro[4.5]dec-3-en-2-yl)methyl)benzonitrile was synthesized from 8-benzoyl-2,3,8-triazaspiro[4.5]dec-3-en-1-one (formula 4.5) through the reaction with the corresponding chemical reagents. ¹H NMR (400 MHz, DMSO) δ7.97 (s, 1H), 7.82 (dd, J=8.3, 1.5 Hz, 2H), 7.56-7.32 (m, 7H), 4.92 (s, 2H), 4.38-4.15 (m, 1H), 3.79-3.58 (m, 1H), 3.55-3.40 (m, 2H), 1.83-1.55 (m, 4H). MS(ESI)m/z: 373.2 (M+H)⁺.

### Compound 70: 8-benzoyl-2-(3-chlorobenzyl)-2,3,8-triazaspiro[4.5]dec-3-en-1-one

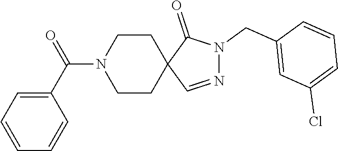 [SMILES: O=C(c1ccccc1)N1CCC2(C=NN(Cc3cccc(Cl)c3)C2=O)CC1]

Similar to the steps described in compound 62, 8-benzoyl-2-(3-chlorobenzyl)-2,3,8-triazaspiro[4.5]dec-3-en-1-one was synthesized from 8-benzoyl-2,3,8-triazaspiro[4.5]dec-3-en-1-one (formula 4.5) through the reaction with the corresponding chemical reagents. ¹H NMR (300 MHz, CDCl₃) δ 7.48-7.39 (m, 5H), 7.38 (s, 1H), 7.29-7.27 (m, 2H), 7.21-7.13 (m, 1H), 4.82 (s, 2H), 4.47-3.84 (m, 2H), 3.81-3.28 (m, 2H), 1.98-1.66 (m, 4H). MS(ESI)m/z: 382.1 (M+H)⁺.

### Compound 71: 8-benzoyl-2-benzyl-2,3,8-triazaspiro[4.5]decan-1-one

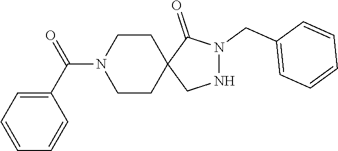 [SMILES: O=C(c1ccccc1)N1CCC2(CC1)CNN(Cc1ccccc1)C2=O]

Similar to the steps described in compound 62, 8-benzoyl-2-benzyl-2,3,8-triazaspiro[4.5]decan-1-one was synthesized from 8-benzoyl-2,3,8-triazaspiro[4.5]dec-3-en-1-one (formula 4.5) through the reaction with the corresponding chemical reagents. ¹H NMR (400 MHz, DMSO) δ 9.28 (s, 1H), 7.50-7.42 (m, 3H), 7.42-7.12 (m, 7H), 4.93 (d, J=15.8 Hz, 1H), 4.57 (d, J=15.8 Hz, 1H), 4.37-4.12 (m, 1H), 3.92-3.75 (m, 1H), 3.69-3.42 (m, 1H), 3.31-3.22 (m, 1H), 3.18-2.97 (m, 2H), 1.73-1.42 (m, 4H). MS(ESI)m/z: 350.2 (M+H)⁺.

### Compound 72: 8-benzoyl-2-(2-fluorobenzyl)-2,3,8-triazaspiro[4.5]decan-1-one

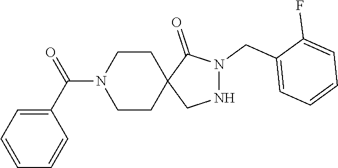 [SMILES: O=C(c1ccccc1)N1CCC2(CC1)CNN(Cc1ccccc1F)C2=O]

Similar to the steps described in compound 62, 8-benzoyl-2-(2-fluorobenzyl)-2,3,8-triazaspiro[4.5]decan-1-one was synthesized from 8-benzoyl-2,3,8-triazaspiro [4.5]dec-3-en-1-one (formula 4.5) through the reaction with the corresponding chemical reagents. ¹H NMR (400 MHz, CDCl₃) δ 7.50-7.35 (m, 5H), 7.32-7.20 (m, 3H), 7.15-6.98 (m, 2H), 4.67 (s, 2H), 4.46-4.37 (m, 1H), 4.37-4.17 (m, 1H), 3.95-3.69 (m, 1H), 3.30-3.11 (m, 3H), 2.02-1.81 (m, 2H), 1.71-1.37 (m, 2H). MS(ESI)m/z: 368.2 (M+H)⁺.

### Compound 73: 7-benzoyl-2-benzyl-2,7-diazaspiro[3.5]nonan-1-one

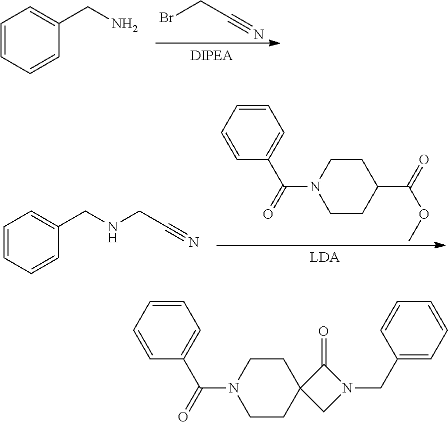

Benzylamine (2.04 mL 18.66 mmol) and bromoacetonitrile (1.3 mL, 18.66 mmol) were dissolved in 50 mL acetonitrile, and DIPEA (6.17 mL, 373.33 mmol) was added and the reaction was stirred at room temperature for 12 h. After the reaction was completed. The solvent was removed under reduced pressure, and the crude product was purified by column chromatography on silica gel to obtain 2-(benzyl amino) acetonitrile.

1-Benzoylpiperidin-4-carboxylate methyl ester (1.6 g, 6.47 mmol) was dissolved in 10 mL THE under a nitrogen atmosphere. LDA (2 M, 6.47 mL, 12.94 mmol) was slowly added at −78° C., and then stirred at −78° C. for 1 hour. 2-(Benzylamino) acetonitrile (946 mg, 6.47 mmol) was dissolved in THE and was added dropwise to the reaction mixture and stirred at −78° C. for 4 h. After the reaction was completed, the solvent was removed under reduced pressure, and the residue was extracted with ethyl acetate. The organic phase was washed with water, and brine, dried over anhydrous Na₂SO₄, and the crude residue was purified by column chromatography on silica gel to provide 7-benzoyl-2-benzyl-2,7-diazaspiro[3.5]nonan-1-one. ¹H NMR (400 MHz, CDCl₃) δ 7.47-7.37 (m, 5H), 7.37-7.28 (m, 3H), 7.26-7.19 (m, 2H), 4.38 (d, J=9.9 Hz, 2H), 4.10-3.90 (m, 1H), 3.87-3.62 (m, 2H), 3.47-3.28 (m, 1H), 3.07-2.88 (m, 2H), 2.01-1.61 (m, 4H). MS(ESI)m/z: 335.2 (M+H)⁺.

### Compound 74: 9-benzoyl-2-benzyl-2,9-diazaspiro[5.5]undecan-1-one

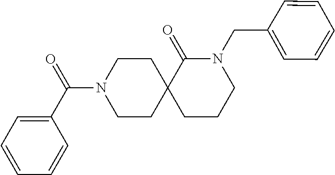 [SMILES: O=C(c1ccccc1)N1CCC2(CCCN(Cc3ccccc3)C2=O)CC1]

Similar to the steps described in compound 9, 9-benzoyl-2-benzyl-2,9-diazaspiro[5.5]undecan-1-one was synthesized from 8-Oxo-3,9-Diazospiro [5.5]undecane 3-carboxylate tert butyl ester through a three steps reaction with the corresponding chemical reagents. ¹H NMR (400 MHz, CDCl₃) δ 7.49-7.37 (m, 5H), 7.37-7.28 (m, 3H), 7.24-7.17 (m, 2H), 4.58 (s, 2H), 4.33-4.15 (m, 1H), 3.91-3.70 (m, 1H), 3.61-3.49 (m, 1H), 3.40-3.27 (m, 1H), 3.27-3.14 (m, 2H), 2.33-2.08 (m, 2H), 1.94-1.79 (m, 4H), 1.73-1.57 (m, 1H), 1.47-1.35 (m, 1H). MS(ESI)m/z: 363.2 (M+H)⁺.

### Compound 75: 2-(2,5-difluorobenzyl)-8-(thiazole-4-carbonyl)-2,8-diazaspiro [4.5]decan-1-one

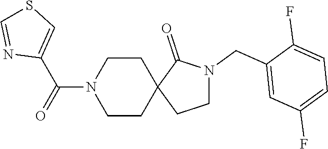 [SMILES: O=C(c1cscn1)N1CCC2(CC1)CCN(Cc1cc(F)ccc1F)C2=O]

Similar to the steps described in compound 9, 2-(2,5-difluorobenzyl)-8-(thiazole-4-carbonyl)-2,8-diazaspiro [4.5]decan-1-one was synthesized from tert-butyl 1-oxo-2,8-diazaspiro [4.5]decane-8-carboxylate (formula 2.3) through a three steps reaction with the corresponding chemical reagents. ¹H NMR (400 MHz, CDCl₃) δ 8.81 (d, J=2.1 Hz, 1H), 7.99 (d, J=2.1 Hz, 1H), 7.08-7.01 (m, 1H), 7.01-6.92 (m, 2H), 4.52 (d, J=6.3 Hz, 2H), 4.48-4.33 (m, 2H), 3.62-3.37 (m, 2H), 3.36-3.25 (m, 2H), 2.11-1.95 (m, 4H), 1.66-1.48 (m, 2H). ¹³C NMR (101 MHz, CDCl₃) δ 177.68, 162.71, 159.07 (d, J=189.0 Hz), 156.66 (d, J=187.8 Hz), 151.87, 151.68, 125.27-124.73 (m), 124.06, 116.83-116.27 (m), 116.14-115.70 (m), 43.58, 42.81, 40.02, 39.41, 33.36, 32.29, 30.45. HRMS(ESI) for C₁₉H₂₀F₂N₃O₂S [M+H]⁺: calcd, 392.1244; found, 392.1247.

### Compound 76: 8-(benzofuran-6-carbonyl)-2-(2,5-difluorobenzyl)-2,8-diazaspiro [4.5]decan-1-one

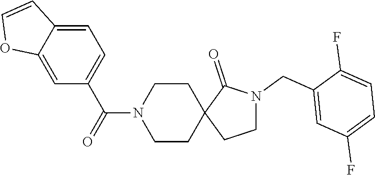 [SMILES: O=C(c1ccc2ccoc2c1)N1CCC2(CC1)CCN(Cc1cc(F)ccc1F)C2=O]

Similar to the steps described in compound 9, 8-(benzofuran-6-carbonyl)-2-(2,5-difluorobenzyl)-2,8-diazaspiro[4.5]decan-1-one was synthesized from 1-oxo-2,8-diazaspiro [4.5]decane-8-carboxylate (formula 2.3) through a three steps reaction with the corresponding chemical reagents. ¹H NMR (400 MHz, DMSO-d6) δ 8.10 (d, J=2.2 Hz, 1H), 7.73 (d, J=7.9 Hz, 1H), 7.65 (s, 1H), 7.33-7.23 (m, 2H), 7.23-7.14 (m, 1H), 7.09-7.00 (m, 2H), 4.43 (s, 2H), 4.41-4.18 (m, 1H), 3.75-3.52 (m, 1H), 3.29-3.08 (m, 4H), 2.00 (d, J=7.8 Hz, 2H), 1.68 (s, 2H), 1.46 (s, 2H). HRMS(ESI) for C₂₄H₂₃F₂N₂O₃[M+H]⁺: calcd, 425.1677; found, 425.1693. HPLC analysis: peak area, 99.2% (254 nm).

### Compound 77: 2-(2,5-difluorobenzyl)-8-(3-methoxybenzoyl)-2,8-diazaspiro[4.5]decan-1-one

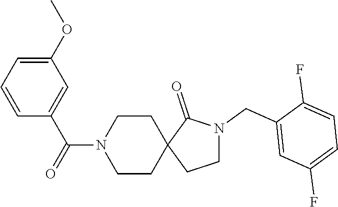 [SMILES: COc1cccc(C(=O)N2CCC3(CC2)CCN(Cc2cc(F)ccc2F)C3=O)c1]

Similar to the steps described in compound 9, 2-(2,5-difluorobenzyl)-8-(3-methoxybenzoyl)-2,8-diazaspiro[4.5]decan-1-one was synthesized from 1-oxo-2,8-diazaspiro [4.5]decane-8-carboxylate (formula 2.3) through a three steps reaction with the corresponding chemical reagents. ¹H NMR (400 MHz, CDCl₃) δ 7.37-7.29 (m, 1H), 7.08-6.89 (m, 6H), 4.52 (s, 2H), 4.50-4.33 (m, 1H), 3.91-3.77 (m, 4H), 3.38-3.13 (m, 4H), 2.14-1.83 (m, 4H), 1.63-1.35 (m, 2H). ¹³C NMR (101 MHz, CDCl₃) δ 177.54, 170.20, 160.00 (d), 159.68, 158.13 (d), 157.57 (d), 155.70 (d), 137.32, 129.61, 125.51-124.41 (m), 118.91, 116.87-116.28 (m), 116.16-115.74 (m), 115.63, 112.08, 55.38, 44.31, 43.43, 42.89, 40.07, 38.89, 33.20, 32.30, 30.19. HRMS(ESI) for C₂₃H₂₅F₂N₃O₄[M+H]⁺: calcd, 415.1833; found, 415.1884. HPLC analysis: peak area, 98.3% (254 nm).

### Example 5

### Kinase Inhibitory Assays

Materials and Instruments: ADP-Glo kinase kit (purchased from Promega), RIPK1 GST-Th-Tag (purchased from Bps Bioscience), MBP substrate (purchased from Millipore), nunc 384-well plate, ATP fluorescence analyzer Paradigm detection platform (purchased from Beckman coulter)

### Methods for the Determination of Activity

The compounds were diluted in DMSO and configured into a solution with a final concentration of 10 mM; Dilute RIP1 kinase from 140 ng/μL to 20 ng/μL for use. Dilute 10 mM of ATP to 100 μM for later use; Dilute 5 mg/mL of MBP to 1 mg/mL for later use. Appropriate embodiments were added into the well of the 384-well plate, 10 μl 100M ATP was evenly mixed with 1 μl 1 mg/mL MB P, the mixed solution was added to the well, and 2 μL 20 ng/L RIP1 kinase was added to the hole, and then incubated at room temperature for 1 h. Subsequently, 5 μL ADP-Glo reagent was added to each well and incubated at room temperature for 40 min. Then, 10 μL ADP-Glo detection reagent was added to each well and incubated at room temperature for 30 min. Finally, the RLU value (relative light unit) of each well was measured by ATP fluorometer, and each sample was repeated twice. Inhibition rate=(RLU value of blank group−RLU value of drug administration group)/RLU value of blank group×100%.

In the embodiments disclosed in this patent, the inhibition of RIPK1 kinase activity was measured in vitro through the above tests, and the inhibition rates of RIPK1 kinase at 10 μM were measured as follows:

| Compound number | Inhibition rate of RIPK1 at 10 μM (%) |
|---|---|
| 2 | 8 |
| 3 | 11 |
| 4 | 16 |
| 5 | 20 |
| 6 | 4 |
| 7 | 34 |
| 8 | 22 |
| 9 | >50 |
| 10 | >50 |
| 11 | >50 |
| 12 | >50 |
| 13 | >50 |
| 14 | >50 |
| 15 | >50 |
| 16 | >50 |
| 17 | 45 |
| 18 | >50 |
| 19 | 16 |
| 20 | 21 |
| 21 | 5 |
| 22 | 8 |
| 23 | >50 |
| 24 | >50 |
| 25 | >50 |
| 26 | 14 |
| 27 | 46 |
| 28 | 3 |
| 29 | >50 |
| 30 | >50 |
| 31 | >50 |
| 32 | >50 |
| 33 | >50 |
| 34 | >50 |
| 35 | >50 |
| 36 | >50 |
| 37 | >50 |
| 38 | >50 |
| 39 | >50 |
| 40 | >50 |
| 41 | >50 |
| 42 | >50 |
| 43 | >50 |
| 44 | >50 |
| 45 | 43 |
| 46 | >50 |
| 47 | >50 |
| 48 | 8 |
| 49 | >50 |
| 50 | >50 |
| 51 | 41 |
| 52 | >50 |
| 53 | >50 |
| 54 | 6 |
| 55 | 13 |
| 56 | 17 |
| 57 | 25 |
| 58 | >50 |
| 59 | 38 |
| 60 | >50 |
| 61 | >50 |
| 62 | >50 |
| 63 | >50 |
| 64 | >50 |
| 65 | 27 |
| 66 | >50 |
| 67 | >50 |
| 68 | 13 |
| 69 | 9 |
| 70 | 44 |
| 71 | 31 |
| 72 | 40 |
| 73 | 45 |
| 74 | >50 |
| 75 | >50 |
| 76 | >50 |
| 77 | >50 |

In the embodiments disclosed in this patent, the inhibition of RIPK1 kinase activity was measured in vitro through the above tests, and the IC₅₀ values measured were as follows:

| Compound number | IC₅₀ (nM) |
|---|---|
| Nec-1 | 1135 |
| GSK2982772 | 32 |
| 31 | 186 |
| 34 | 693 |
| 42 | 1044 |
| 44 | 186 |
| 46 | 624 |
| 49 | 92 |
| 50 | 102 |

### Example 6

### Cell Anti-Necroptosis Activity Assay:

Materials: U937 cell line (human myeloid leukemia cells), RPMI 1640 medium (purchased from Gibco, USA), penicillin-streptomycin double antibody (purchased from Gibco, USA), fetal bovine serum (purchased from genetic, USA), tetradecanoylphorbol acetate (purchased from biyuntian Biotechnology Co., Ltd.), CCK8 cell proliferation detection kit (purchased from biyuntian Biotechnology Co., Ltd), Z-VAD-FMK (from APExBIO, USA) and LPS (from Sigma, USA).

U937 cells were cultured in RPMI 1640 containing 10% heat inactivated fetal bovine serum and 1% penicillin-streptomycin in a constant temperature cell incubator at 37° C. and 5% CO₂, and subcultured every 1-2 days. Human monocytic cell line U937 cells could be induced to differentiate into mature monocytes/macrophages after stimulation with tetradecanoylphorbol acetate (PMA). The cells in the logarithmic growth phase were seeded in 96 well plates at a density of 4×10⁴ cells/well and cultured. PMA 100 ng/ml was added for 24 h, and then the fresh medium was changed. The necroptosis model was constructed by stimulation of LPS and caspase inhibitor Z-VAD-FMK. In the experimental groups, LPS (100 ng/ml) was added for 1 hour, and then the solvent control (DMSO) or different concentrations of compounds to be tested (30 μM, 10 μM, 3 μM, 1 μM, 300 nM, 100 nM, 30 nM 7 concentrations in total) for 1 h, and then the experimental groups were treated with caspase inhibitor Z-VAD-FMK (20 μM). The cell viability was detected after 24 h of culture. The control groups were not treated with LPS, drugs and Z-VAD-FMK. No cells were added to the blank hole, only the culture medium. Each group is set with 4 parallel. CCK8 cell proliferation assay kit was used to detect the cell viability and survival rate, and the operation was carried out according to the kit instructions. The cell supernatant was added with 10 μL CCK8 detection reagent, and the cell absorbance (450 nm) was detected by a microplate reader after 0.5 h. Calculate the cell death rate (%)=(average absorbance of control well−average absorbance of experimental group)/(average absorbance of control well−average absorbance of the blank well)×100. Finally, the half effective concentration (EC50) of the drug was calculated by GraphPad Prism 7.0® software.

The inhibitory effect of the embodiments disclosed in this patent on necroptosis in vitro was determined by the above tests, and the measured EC₅₀ values were as follows:

| Compound number | EC₅₀ (nM) |
|---|---|
| 31 | 1048 |
| 34 | 1338 |
| 42 | 1652 |
| 44 | 161 |
| 46 | 1196 |
| 49 | 1110 |
| 50 | 1560 |

### Example 7

### Pharmacodynamic Study of Compound 31 in the Treatment of Ischemic Stroke

Animals: SPF-grade male Sprague Dawley (SD) rats (purchased from Beijing Weitonglihua Experimental Animal Technology Co., Ltd.). Rats were fed with a free diet, 12 h: 12 h circadian rhythm.

Material: 2,3,5-Triphenyltetrazolium chloride (purchased from Beijing Solebo Technology Co., Ltd.), N,N-Dimethylacetamide (purchased from Shanghai Aladdin Biochemical Technology Co., Ltd.), castor oil polyoxyethylene ether (purchased from Shanghai McLean Biochemical Technology Co., Ltd.), sodium chloride (purchased from Bioengineering (Shanghai) Co., Ltd.), isoflurane (purchased from Shenzhen ruiwode Life Technology Co., Ltd.), and Sodium dihydrogen phosphate (purchased from Sinopharm group), disodium hydrogen phosphate (purchased from Sinopharm group), paraformaldehyde (purchased from biotechnology (Shanghai) Co., Ltd.) and thread bolt (purchased from Beijing Xinong Technology Co., Ltd.).

Methods: A rat model of ischemic stroke was established by middle cerebral artery occlusion (MCAO) of cerebral ischemia/reperfusion: Healthy male SD rats (weighing 260±40 g) were anesthetized with 5% isoflurane and maintained with 1.5%-2% isoflurane. The rats were fixed in the supine position, and the right common carotid artery, internal carotid artery and external carotid artery were separated through the median neck incision and threaded for standby. The proximal end of the common carotid artery and the external carotid artery were ligated, and the internal carotid artery was clamped with an artery clamp. The suture plug was cut and inserted at about 0.5 cm from the bifurcation of the common carotid artery. The artery clamp was released, and the suture plug was delivered until the blood flow of the middle cerebral artery was blocked. After 60 minutes of ischemia, the suture plug was removed to restore blood flow. The rectal temperature was maintained at 37±0.5° C. during the operation. After 48 hours of ischemia/reperfusion, the neurological function was evaluated by modified neurological severity score (MNSs). The cerebral infarction volume was detected by TTC staining, and the brain water content was detected to evaluate the cerebral edema.

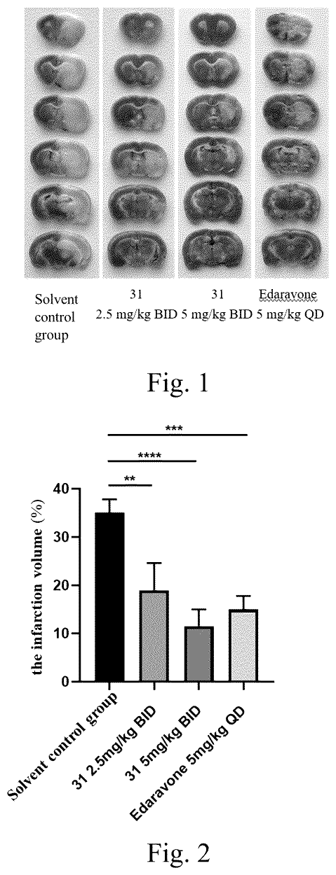

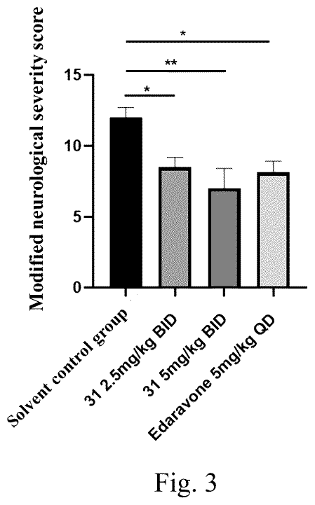

The pharmacodynamic study of the embodiments disclosed in this patent in the treatment of brain was determined by the above tests, and the measured results were shown in FIG. 1, FIG. 2 and FIG. 3.

In conclusion, compound 31 2.5 mg/kg and 5 mg/kg bid could significantly reduce the infarct volume, nerve injury score and water content in the ischemic side of tMCAO. Compared with the positive control edaravone 5 mg/kg QD group, compound 31 at 5 mg/kg bid had stronger protective effect on cerebral ischemia injury in the tMCAO model. The results suggested that compound 31 had a good therapeutic effect on ischemic stroke.

1. A compound, which is a spirocyclic compound having a structure of Formula (I), or a pharmaceutically acceptable salt, or a stereoisomer thereof: 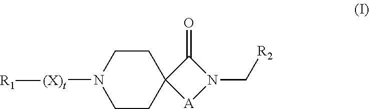
wherein, A is C₁-C₃ alkylene, —CH₂—NH— [symbol] —CH═N— [symbol] 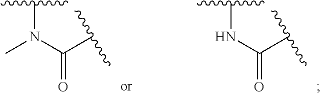
is 1;
X is —CO—, —SO₂—, —(CH₂)ₙCO—, —(CH₂)˜SO₂—, or —NHCO—, wherein, n is 1, 2, 3 or 4;
R₁ is:
1) H; or
2) substituted and unsubstituted C₆-C₁₀ aryl and 4-10 membered heteroaryl; or
3) C₁-C₄ alkyl, C₃-C₇ cycloalkyl, substituted and unsubstituted 3-8 membered heterocyclyl;
in R₁, the substituted 3-8 membered heterocyclyl and the substituted 4-10 membered heteroaryl is independently substituted by substituents selected from the group consisting of halogen, amino, nitro, trifluoromethyl, difluoromethyl, cyano, hydroxyl, —C(O)C₁-C₆ alkyl, C₁-C₆ alkyl, C₁-C₆ haloalkyl, C₁-C₆ alkoxy, and C₁-C₆ haloalkoxy;
in R₁, the substituted C₆-C₁₀ aryl is independently substituted by substituents selected from the group consisting of amino, nitro, trifluoromethyl, difluoromethyl, cyano, hydroxyl, —C(O)C₁-C₆ alkyl, C₁-C₆ alkyl, C₁-C₆ haloalkyl, C₁-C₆ alkoxy, and C₁-C₆ haloalkoxy;
R₂ is:
1) substituted and unsubstituted C₆-C₁₀ aryl, substituted and unsubstituted 4-10 membered heteroaryl;
2) C₁-C₄ alkyl, C₃-C₇ cycloalkyl, substituted and unsubstituted 3-8 membered heterocyclyl;
in R₂, the substituted 3-8 membered heterocyclyl, substituted C₆-C₁₀ aryl or substituted 4-10 membered heteroaryl is independently substituted by substituents selected from the group consisting of halogen, amino, nitro, trifluoromethyl, difluoromethyl, cyano, hydroxyl, —C(O)C₁-C₆ alkyl, C₁-C₆ alkyl, C₁-C₆ haloalkyl, C₁-C₆ alkoxy, and C₁-C₆ haloalkoxy.

2. (canceled)

3. The compound according to claim 1, wherein,
R₁ is C₆-C₈ aryl or 5-8 membered heteroaryl; or
X is —CO—, —SO₂—, or C₁-C₄ alkyl; or
R₂ is substituted and unsubstituted C₆-C₁₀ aryl, substituted and unsubstituted 4-10 membered heteroaryl, or C₃-C₇ cycloalkyl.

4. The compound according to claim 3, wherein, the compound is any one of the following structures: 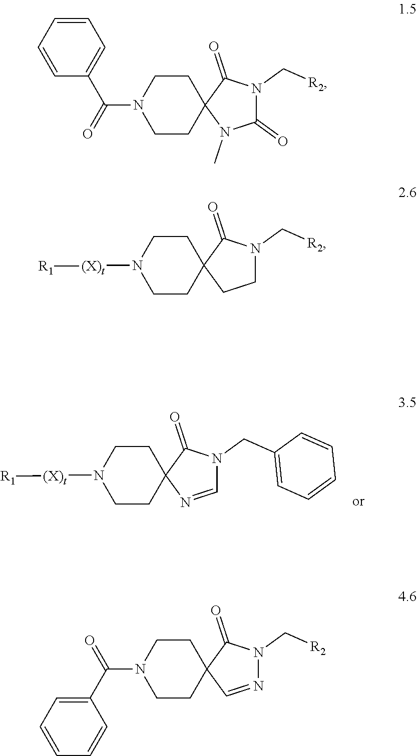
wherein,
X is —CO—, or —SO₂—; or
R₁ is substituted and unsubstituted C₆-C₈ aryl, or substituted and unsubstituted 5-8 membered heteroaryl; or
R₂ is substituted and unsubstituted C₆-C₁₀ aryl, substituted and unsubstituted 4-10 membered heteroaryl, or substituted and unsubstituted C₃-C₇ cycloalkyl.

5. A compound, or a pharmaceutically acceptable salt, or a stereoisomer thereof, wherein the compound is selected from:

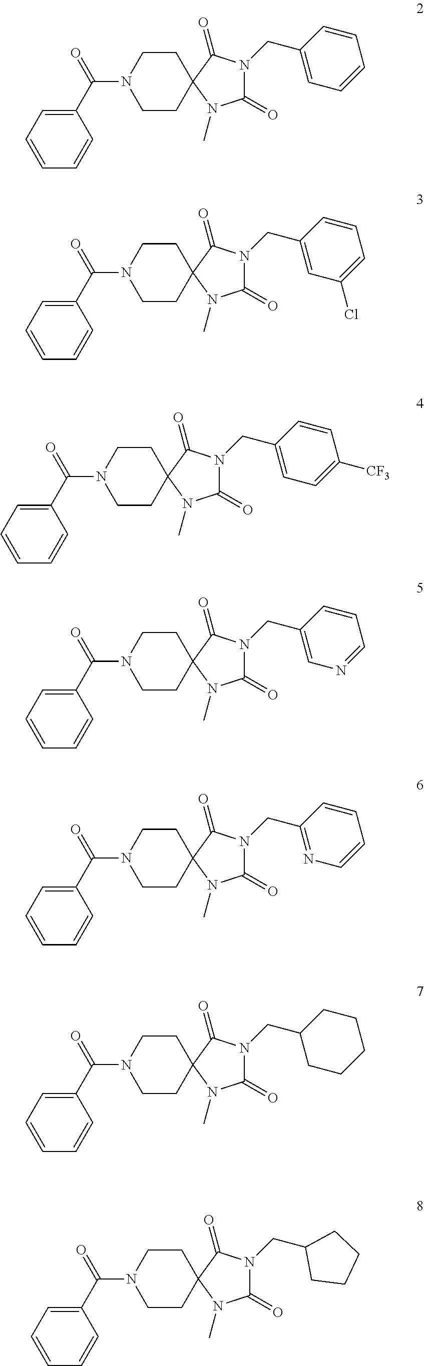

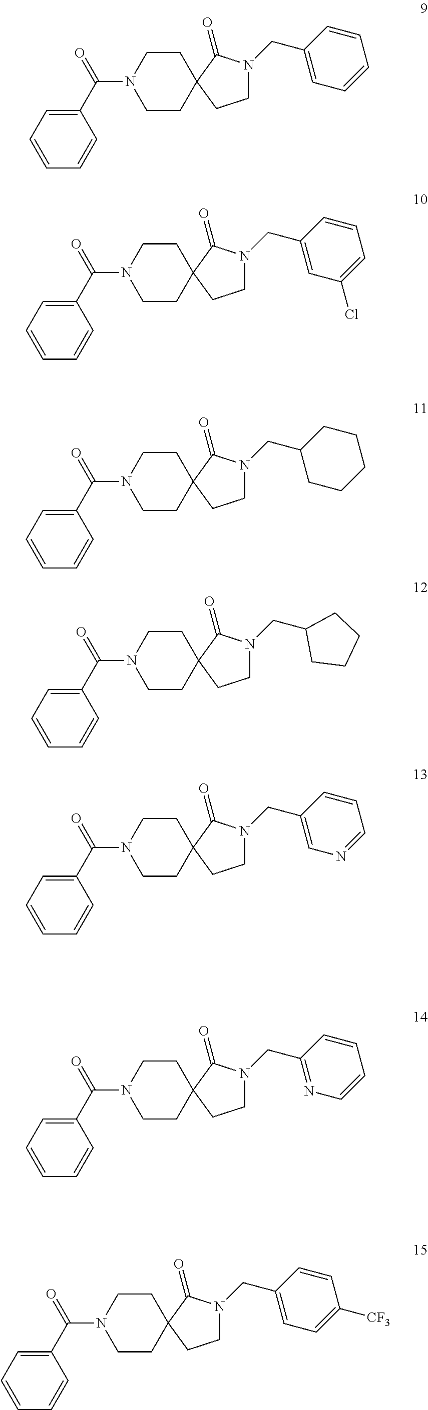

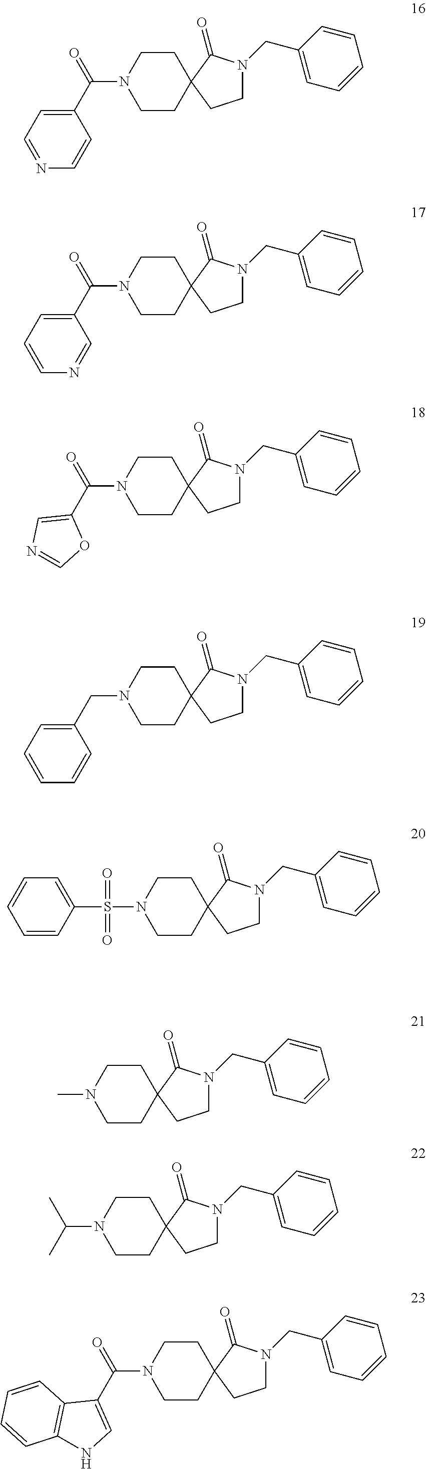

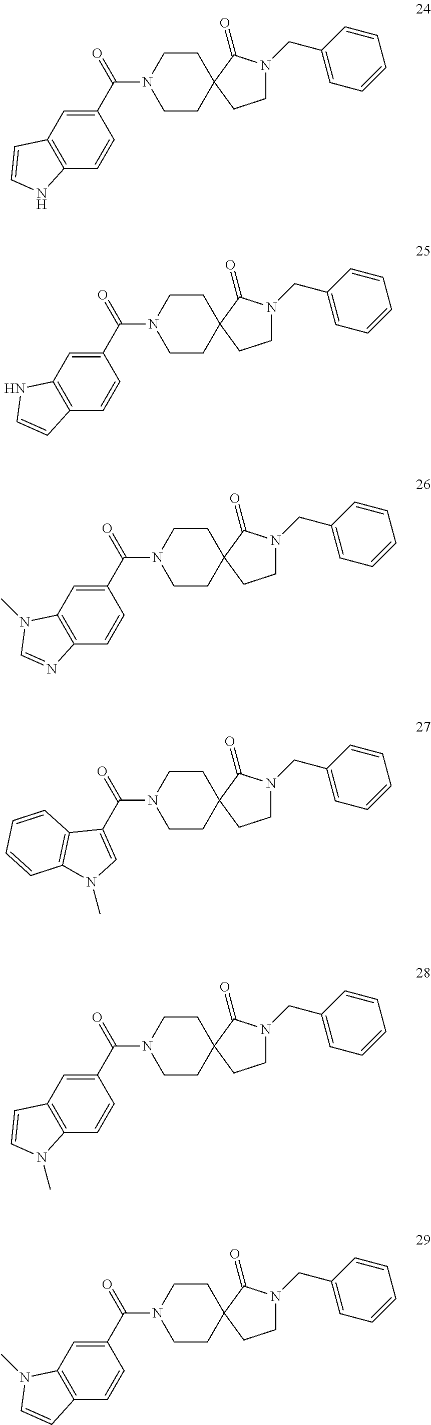 [SMILES: O=C(c1ccc2[nH]ccc2c1)N1CCC2(CC1)CCN(Cc1ccccc1)C2=O ; O=C(c1ccc2cc[nH]c2c1)N1CCC2(CC1)CCN(Cc1ccccc1)C2=O ; Cn1cnc2ccc(C(=O)N3CCC4(CC3)CCN(Cc3ccccc3)C4=O)cc21 ; Cn1cc(C(=O)N2CCC3(CC2)CCN(Cc2ccccc2)C3=O)c2ccccc21 ; Cn1ccc2cc(C(=O)N3CCC4(CC3)CCN(Cc3ccccc3)C4=O)ccc21 ; Cn1ccc2ccc(C(=O)N3CCC4(CC3)CCN(Cc3ccccc3)C4=O)cc21]

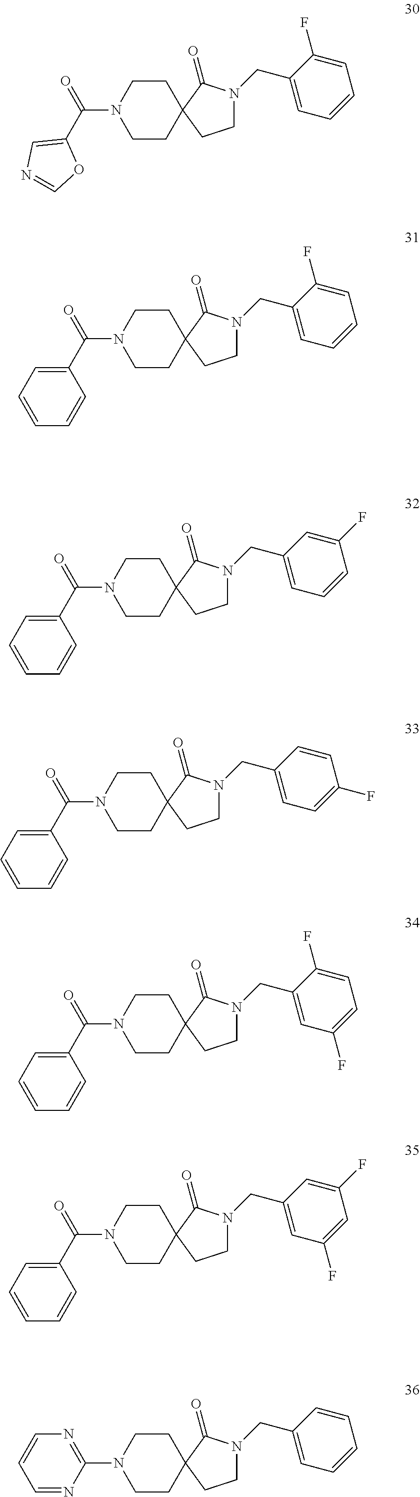

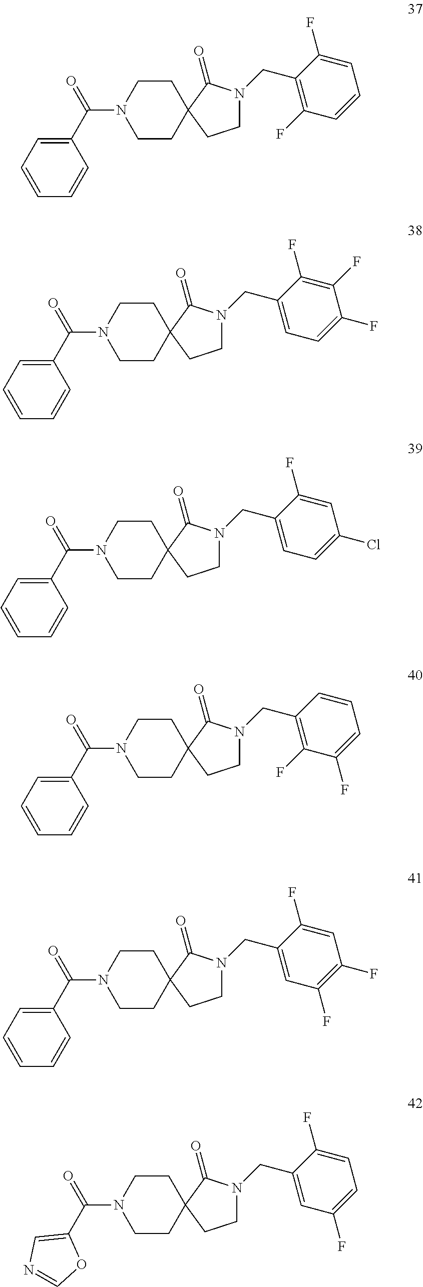 [SMILES: O=C(c1ccccc1)N1CCC2(CC1)CCN(Cc1c(F)cccc1F)C2=O ; O=C(c1ccccc1)N1CCC2(CC1)CCN(Cc1ccc(F)c(F)c1F)C2=O ; O=C(c1ccccc1)N1CCC2(CC1)CCN(Cc1ccc(Cl)cc1F)C2=O ; O=C(c1ccccc1)N1CCC2(CC1)CCN(Cc1cccc(F)c1F)C2=O ; O=C(c1ccccc1)N1CCC2(CC1)CCN(Cc1cc(F)c(F)cc1F)C2=O ; O=C(c1cnco1)N1CCC2(CC1)CCN(Cc1cc(F)ccc1F)C2=O]

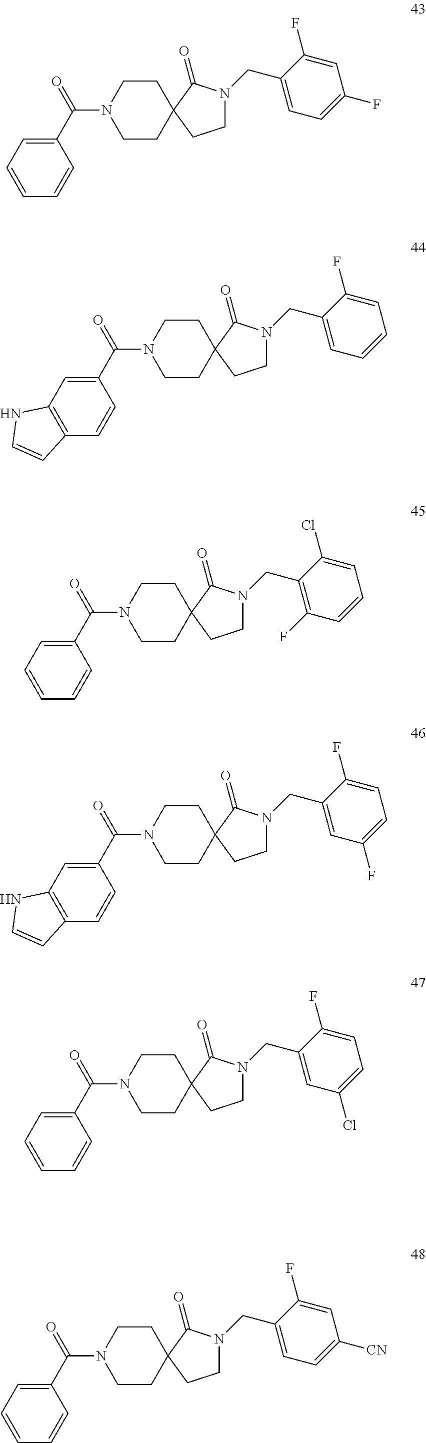 [SMILES: O=C(c1ccccc1)N1CCC2(CC1)CCN(Cc1ccc(F)cc1F)C2=O ; O=C(c1ccc2cc[nH]c2c1)N1CCC2(CC1)CCN(Cc1ccccc1F)C2=O ; O=C(c1ccccc1)N1CCC2(CC1)CCN(Cc1c(F)cccc1Cl)C2=O ; O=C(c1ccc2cc[nH]c2c1)N1CCC2(CC1)CCN(Cc1cc(F)ccc1F)C2=O ; O=C(c1ccccc1)N1CCC2(CC1)CCN(Cc1cc(Cl)ccc1F)C2=O ; N#Cc1ccc(CN2CCC3(CCN(C(=O)c4ccccc4)CC3)C2=O)c(F)c1]

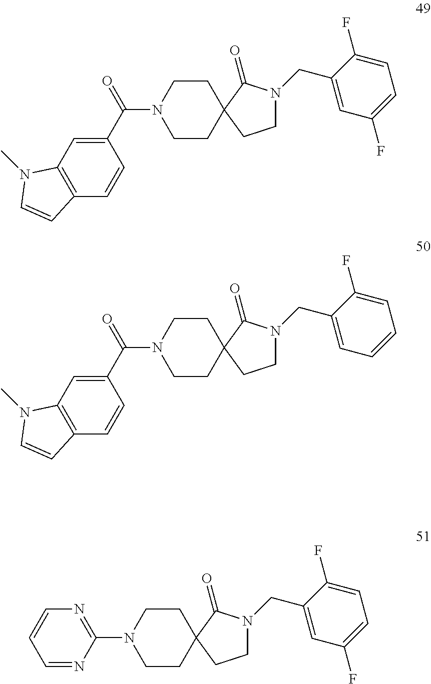 [SMILES: Cn1ccc2ccc(C(=O)N3CCC4(CC3)CCN(Cc3cc(F)ccc3F)C4=O)cc21 ; Cn1ccc2ccc(C(=O)N3CCC4(CC3)CCN(Cc3ccccc3F)C4=O)cc21 ; O=C1N(Cc2cc(F)ccc2F)CCC12CCN(c1ncccn1)CC2]

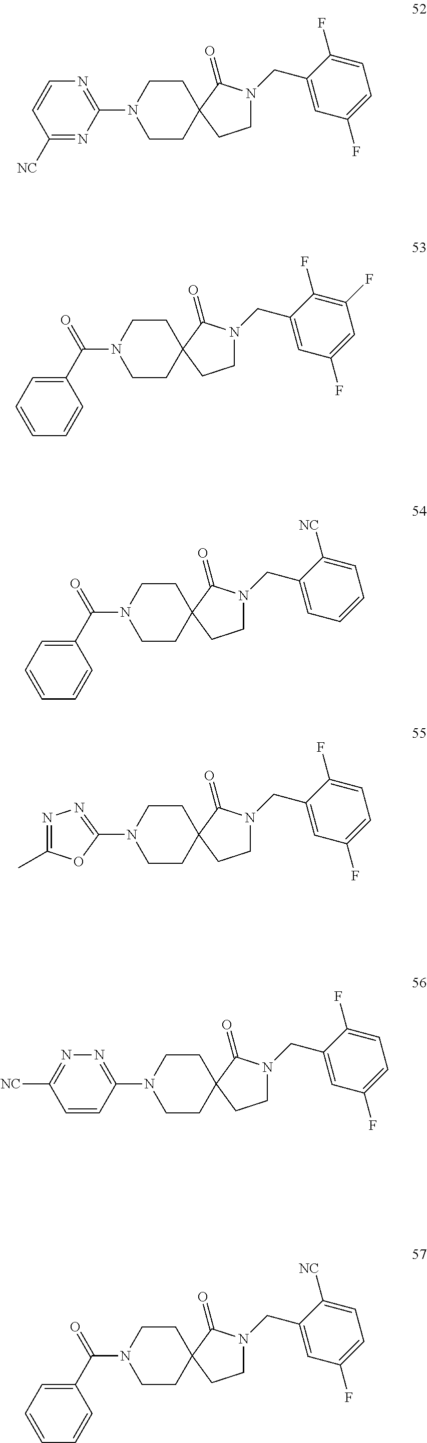 [SMILES: N#Cc1ccnc(N2CCC3(CCN(Cc4cc(F)ccc4F)C3=O)CC2)n1 ; O=C(c1ccccc1)N1CCC2(CC1)CCN(Cc1cc(F)cc(F)c1F)C2=O ; N#Cc1ccccc1CN1CCC2(CCN(C(=O)c3ccccc3)CC2)C1=O ; Cc1nnc(N2CCC3(CCN(Cc4cc(F)ccc4F)C3=O)CC2)o1 ; N#Cc1ccc(N2CCC3(CCN(Cc4cc(F)ccc4F)C3=O)CC2)nn1 ; N#Cc1ccc(F)cc1CN1CCC2(CCN(C(=O)c3ccccc3)CC2)C1=O]

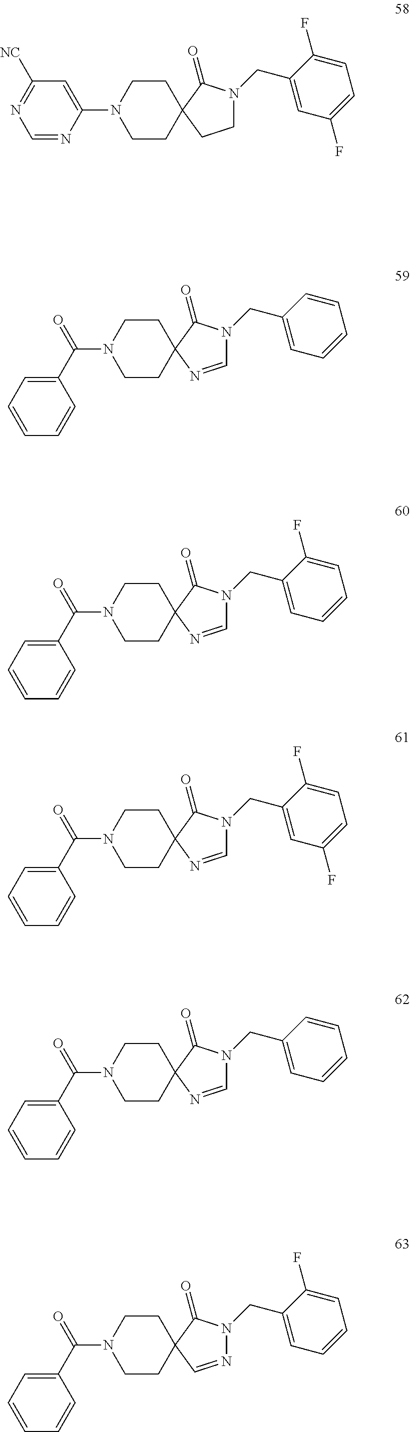 [SMILES: N#Cc1cc(N2CCC3(CCN(Cc4cc(F)ccc4F)C3=O)CC2)ncn1 ; O=C(c1ccccc1)N1CCC2(CC1)N=CN(Cc1ccccc1)C2=O ; O=C(c1ccccc1)N1CCC2(CC1)N=CN(Cc1ccccc1F)C2=O ; O=C(c1ccccc1)N1CCC2(CC1)N=CN(Cc1cc(F)ccc1F)C2=O ; O=C(c1ccccc1)N1CCC2(CC1)N=CN(Cc1ccccc1)C2=O ; O=C(c1ccccc1)N1CCC2(C=NN(Cc3ccccc3F)C2=O)CC1]

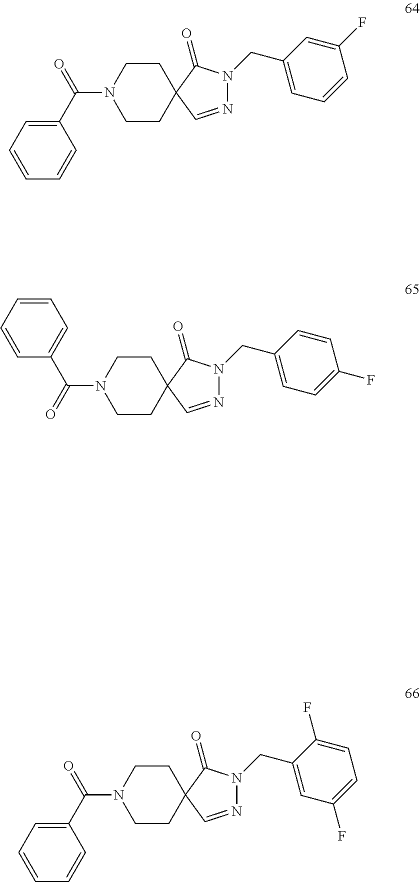 [SMILES: O=C(c1ccccc1)N1CCC2(C=NN(Cc3cccc(F)c3)C2=O)CC1 ; O=C(c1ccccc1)N1CCC2(C=NN(Cc3ccc(F)cc3)C2=O)CC1 ; O=C(c1ccccc1)N1CCC2(C=NN(Cc3cc(F)ccc3F)C2=O)CC1]

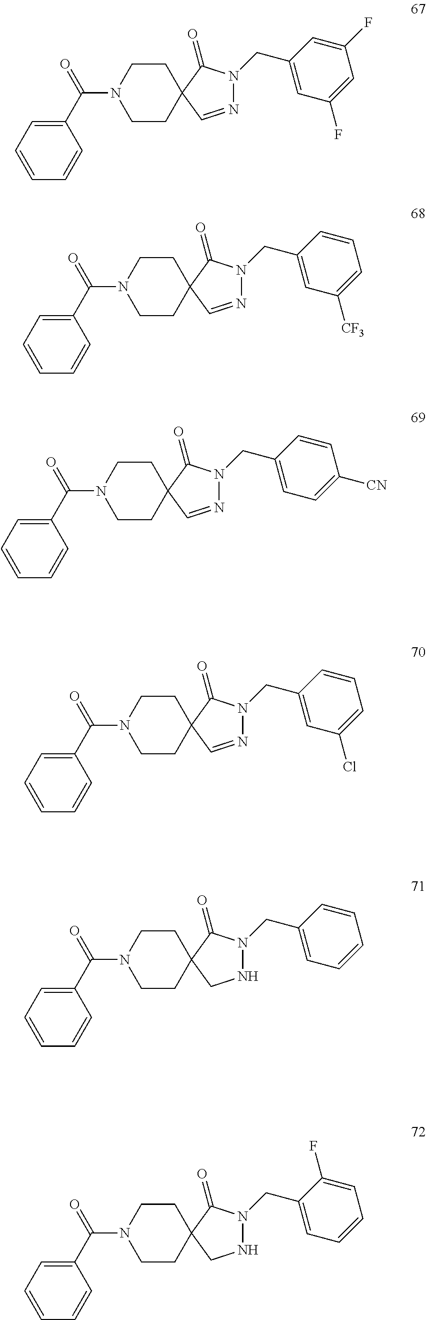 [SMILES: O=C(c1ccccc1)N1CCC2(C=NN(Cc3cc(F)cc(F)c3)C2=O)CC1 ; O=C(c1ccccc1)N1CCC2(C=NN(Cc3cccc(C(F)(F)F)c3)C2=O)CC1 ; N#Cc1ccc(CN2N=CC3(CCN(C(=O)c4ccccc4)CC3)C2=O)cc1 ; O=C(c1ccccc1)N1CCC2(C=NN(Cc3cccc(Cl)c3)C2=O)CC1 ; O=C(c1ccccc1)N1CCC2(CC1)CNN(Cc1ccccc1)C2=O ; O=C(c1ccccc1)N1CCC2(CC1)CNN(Cc1ccccc1F)C2=O]

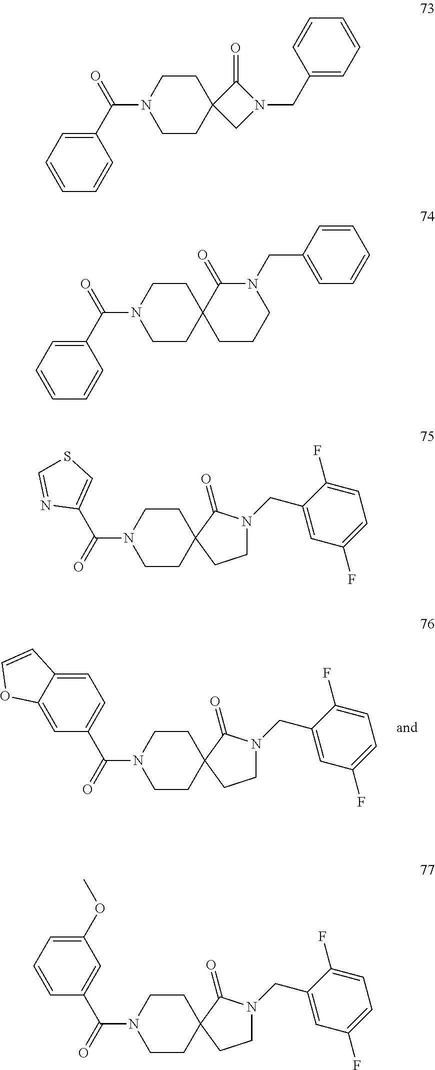 [SMILES: O=C(c1ccccc1)N1CCC2(CC1)CN(Cc1ccccc1)C2=O ; O=C(c1ccccc1)N1CCC2(CCCN(Cc3ccccc3)C2=O)CC1 ; O=C(c1cscn1)N1CCC2(CC1)CCN(Cc1cc(F)ccc1F)C2=O ; O=C(c1ccc2ccoc2c1)N1CCC2(CC1)CCN(Cc1cc(F)ccc1F)C2=O ; COc1cccc(C(=O)N2CCC3(CC2)CCN(Cc2cc(F)ccc2F)C3=O)c1]

6. A preparation method for the compound according to claim 1, wherein, the preparation method comprises the following steps: 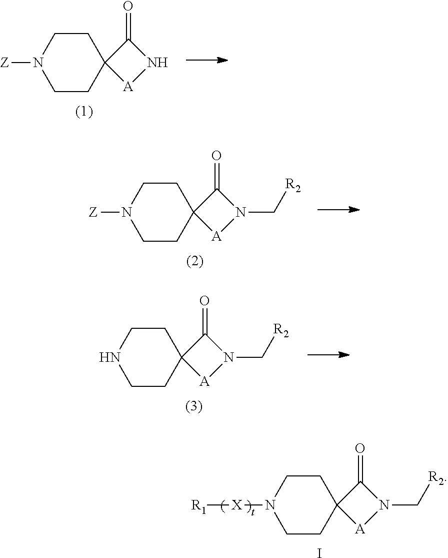
Z is Boc, Cbz, Fmoc, Trt, or Alloc;
A. raw material (1) reacts with a halomethyl-substituted C₆-C₁₀ aryl, a halomethyl-substituted 4-10 membered heteroaryl, a C₃-C₇ cycloalkyl in an organic solvent with base as catalyst at room temperature to obtain intermediate (2) by conventional separation and purification;
B. intermediate (2) undergoes deprotection of the protective group Z by reacting with an acid in an organic solvent to obtain intermediate (3);
C. in an organic solvent, in the presence of a condensation agent and an organic base, intermediate (3) reacts with a halogen-substituted C₆-C₁₀ aryl, a halogen-substituted 4-10 membered heteroaryl, a carboxyl-substituted C₆-C₁₀ aryl, or a carboxyl-substituted 4-10 membered heteroaryl to prepare the compound shown in general Formula I.

7-9. (canceled)

10. The compound according to claim 1, wherein,
the alkyl refers to an unbranched or branched saturated hydrocarbon chain;
or, the haloalkyl refers to an unbranched or branched alkyl, wherein one or more hydrogen atoms are replaced by a halogen;
or, the alkoxy refers to alkyl-O—;
or, the heterocyclyl refers to a saturated or partially unsaturated cyclic hydrocarbon group, with one or more ring heteroatoms independently selected from nitrogen, oxygen and sulfur;
or, the heteroaryl refers to a heteroaryl system consisting of 1-4 heteroatoms, including nitrogen, oxygen and sulfur.

11. The compound according to claim 10, wherein,
the alkyl is selected from methyl, ethyl, n-propyl, isopropyl, n-butyl, isobutyl, tert-butyl, sec-butyl, and n-pentyl;
or, 0 haloalkyl is selected form monofluoromethyl, monochloroethyl, difluoromethyl, dichloromethyl, dibromomethyl, trifluoromethyl, trichloromethyl, and tribromomethyl;
or, the alkoxy is selected from methoxy, ethoxy, propoxy, and butoxy;
or, the aryl is selected from phenyl, and naphthyl;
or, the heteroaryl is selected form furanyl, thienyl, pyridinyl, pyrrolyl, N-alkyl pyrrolyl, pyrimidinyl, pyrazinyl, imidazolyl, and tetrazolyl.

12. The compound according to claim 10, wherein, X is —CO—.

13. The compound according to claim 10, wherein, R₁ is phenyl.

14. A pharmaceutical composition, comprising the compound, the pharmaceutically acceptable salt, or the stereoisomer thereof according to claim 1, and a pharmaceutically acceptable carrier;
the pharmaceutically acceptable carrier is selected from a filler, a lubricant, an emulsifier, a wetting agent, a colorant, a flavoring, a stabilizer, an antioxidant, and a preservative.

15. A pharmaceutical composition, comprising the compound, the pharmaceutically acceptable salt, or the stereoisomer thereof according to claim 5, and a pharmaceutically acceptable carrier;
the pharmaceutically acceptable carrier is selected from a filler, a lubricant, an emulsifier, a wetting agent, a colorant, a flavoring, a stabilizer, an antioxidant, and a preservative.

16. A method for inhibiting RIPK1 kinase activity in a subject in need thereof, comprising administering the compound, the pharmaceutically acceptable salt, or the stereoisomer thereof according to claim 1.

17. A method for inhibiting RIPK1 kinase activity in a subject in need thereof, comprising administering the compound, the pharmaceutically acceptable salt, or the stereoisomer thereof according to claim 5.

18. A method for preventing and/or treating RIPK1-related disease in a subject in need thereof, comprising administering the compound, the pharmaceutically acceptable salt, or the stereoisomer thereof according to claim 1.

19. The method of claim 18, wherein,
the RIPK1-related disease is tumor, ischemic stroke, rheumatoid arthritis, amyotrophic lateral sclerosis, multiple sclerosis, autoimmune disease, neurodegenerative disease, alcoholic steatohepatitis, non-alcoholic steatohepatitis, systemic inflammatory response syndrome, inflammatory bowel disease, or psoriasis.

20. A method for preventing and/or treating RIPK1-related disease in a subject in need thereof, comprising administering the compound, the pharmaceutically acceptable salt, or the stereoisomer thereof according to claim 5.

21. The method of claim 20, wherein,
the RIPK1-related disease is tumor, ischemic stroke, rheumatoid arthritis, amyotrophic lateral sclerosis, multiple sclerosis, autoimmune disease, neurodegenerative disease, alcoholic steatohepatitis, non-alcoholic steatohepatitis, systemic inflammatory response syndrome, inflammatory bowel disease, or psoriasis.
